(19) United States
(12) Patent Application Publication
(10) Pub. No.: US 2024/0336638 A1
(43) Pub. Date: Oct. 10, 2024

(54) IMIDAZOLE COMPOUND, AND INTERMEDIATE AND APPLICATION THEREOF

(71) Applicants: Guoqin ZHUGE, Jiangsu (CN); Xiaoqin LI, Jiangsu (CN)

(72) Inventors: Xiaoqin LI, Jiangsu (CN); Jianfang CHEN, Jiangsu (CN)

(21) Appl. No.: 18/571,763

(22) PCT Filed: Jun. 28, 2022
(86) PCT No.: PCT/CN2022/102041

§ 371 Date: Dec. 19, 2023

(30) Foreign Application Priority Data
Jul. 1, 2021 (CN) 202110744200.8

(51) Int. Cl. C07F 7/18; A61K 31/216; A61K 31/4178; A61K 31/4439; A61K 31/501; A61K 31/506; A61K 31/695; A61P 7/02; A61P 9/10; C07C 69/757; C07D 213/16; C07D 233/58; C07D 233/68; C07D 401/06; C07D 401/14; C07D 403/06; C07D 403/10

(52) U.S. Cl. CPC C07F 7/1804; A61K 31/216; A61K 31/4178; A61K 31/4439; A61K 31/501; A61K 31/506; A61K 31/695; A61P 7/02; A61P 9/10; C07C 69/757; C07D 213/16; C07D 233/58; C07D 233/68; C07D 401/06; C07D 401/14; C07D 403/06; C07D 403/10

(57) ABSTRACT

An imidazole compound as shown in formula I or a pharmaceutically acceptable salt and an intermediate thereof. The imidazole compound as shown in formula I can significantly inhibit AA-induced platelet aggregation, improves MCAO/R-induced rat cerebral ischemia injury, has excellent metabolic stability, and can improve the distribution of a drug within the brain tissue, such that the pharmacodynamic activity of the imidazole compound in treatment of dyskinesia associated with acute thrombotic cerebral infarction and cerebral infarction is improved.

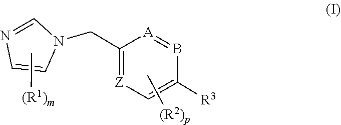

The present application claims the right of the priority of Chinese patent application 2021107442008 filed on Jul. 1, 2021. The contents of the above Chinese patent application are incorporated herein by reference in their entireties.

### TECHNICAL FIELD

The present disclosure relates to an imidazole compound, an intermediate thereof, and a use thereof.

### BACKGROUND

With the development of human society and the increasing aging of the population, deaths caused by thrombotic diseases currently account for 52% of the total deaths worldwide. The number of patients with thrombotic diseases continues to increase, and the incidence of thrombotic diseases such as myocardial infarction and cerebral thrombosis is on the rise, seriously threatening people's health.

Thrombus refers to the coagulation of blood components (platelets, coagulation factors) in the human body in blood vessels or heart chambers to form abnormal blood clots. Thrombus formed due to slow blood flow, abnormal blood components, or increased blood viscosity can lead to acute myocardial infarction, pulmonary embolism, and other diseases of the heart, brain, and pulmonary circulation. It is also a common complication in surgical operations, threatening human life. The formation mechanism of thrombus and the factors influencing thrombus formation are quite complex. The formation of thrombosis is mainly related to six factors: (1) changes in the vascular wall; (2) changes in the tunica intima of the vascular wall; (3) changes in blood flow speed; (4) changes in platelets; (5) changes in the state of blood coagulation; (6) changes in hemorheological factors, etc.

Antithrombotic drugs typically include antiplatelet drugs, anticoagulants, and thrombolytic drugs. TXA₂ (Thromboxane A2) is a bioactive substance produced by platelets that strongly constricts blood vessels and causes platelet aggregation. Prostaglandin G₂ (PGG₂) and prostaglandin H₂ (PGH₂) form TXA₂ under the action of TXA₂ synthase. The pharmacological mechanism of TXA₂ synthase inhibitors is to inhibit platelet aggregation by inhibiting TXA₂ synthase.

Ozagrel is the world's first marketed potent thromboxane A₂ (TXA₂) synthase inhibitor. It is an antithrombotic drug first marketed in 1988 through joint research by Ono and Kissei Pharmaceutical Co., Ltd. of Japan and is available in two medicinal forms, sodium salt (CAS: 189224-26-8) and monohydrochloride salt (CAS: 78712-43-3). The active ingredient of its sodium salt form was initially marketed under the trade name Xanbao and commonly used to treat acute thrombotic cerebral infarction and dyskinesia associated with cerebral infarction; its monohydrochloride salt is used to treat bronchial asthma and angina pectoris.

The antithrombotic drug Ozagrel is widely used in clinical practice with clear efficacy, but the metabolic stability of this active ingredient is poor and its distribution in brain tissue is limited.

### CONTENT OF THE PRESENT INVENTION

The existing antithrombotic drug Ozagrel has poor metabolic stability and low distribution in brain tissue. For this reason, the present disclosure provides an imidazole compound, an intermediate thereof, and a use thereof. The imidazole compound of formula I of the present disclosure is based on the antithrombotic drug Ozagrel as a precursor. Through structural modification, its drug-like properties are improved, especially increasing the distribution of the drug in brain tissue, thereby enhancing its pharmacodynamic activity in the treatment of acute thrombotic cerebral infarction and dyskinesia associated with cerebral infarction.

The present disclosure provides a compound of formula I or a pharmaceutically acceptable salt thereof;

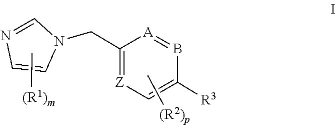

wherein A, B, and Z are independently CH or N;
R¹ and R² are each independently H, halogen, or C₁₋₆ alkyl;
m is 0, 1, 2, or 3;
R³ is

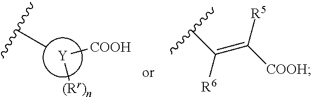

ring Y is a 3- to 6-membered cycloalkyl ring or a 3- to 6-membered heterocycloalkyl ring containing 1 to 3 heteroatoms selected from one or more than one of N, O, and S;
p and n are independently 0, 1, 2, 3, or 4;
each R^{r} is independently H, —OH, halogen, C₁₋₆ alkyl, or C₁₋₆ alkoxy;
R⁵ and R⁶ are independently H, C₂₋₆ alkenyl, C₂₋₆ alkynyl, C₂₋₆ alkynyl substituted by one or more than one R⁵⁻¹, 5- to 6-membered heteroaryl, or 5- to 6-membered heteroaryl substituted by one or more than one R⁵⁻², and the 5- to 6-membered heteroaryl in the 5- to 6-membered heteroaryl and 5- to 6-membered heteroaryl substituted by one or more than one R⁵⁻² contains 1 to 4 heteroatoms selected from one or more than one of N, O, or S; when there is more than one substituent, the substituents are the same or different;
R⁵⁻¹ and R⁵⁻² are independently halogen, C₁₋₆ alkyl, or C₁₋₆ alkoxy;
when A, B, and Z are CH at the same time, R⁵ and R⁶ are not H at the same time.

In a certain embodiment, in the imidazole compound of formula I or the pharmaceutically acceptable salt thereof, certain groups have the following definitions, and the definitions of unmentioned groups are as described in any embodiment of the present disclosure (this paragraph is hereinafter referred to as “in a certain embodiment”),

A, B, and Z are all CH, or at least one selected from the group of A, B, and Z is N.

In a certain embodiment, R¹ is H or halogen.

In a certain embodiment,

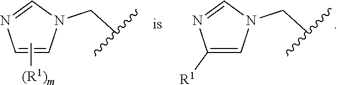

In a certain embodiment, m, p, and n are independently 0 or 1.

In a certain embodiment, when cis-trans isomerism of

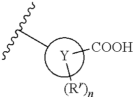

is present, the

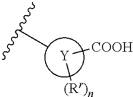

is of trans configuration.

In a certain embodiment,

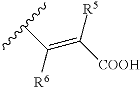

is of trans configuration.

In a certain embodiment, R^{r} is independently H or —OH.

In a certain embodiment, R⁵ is independently H, C₂₋₆ alkynyl, C₂₋₆ alkynyl substituted by one or more than one R⁵⁻¹, or 5- to 6-membered heteroaryl containing 1 to 2 N heteroatoms.

In a certain embodiment, R⁶ is H.

In a certain embodiment,

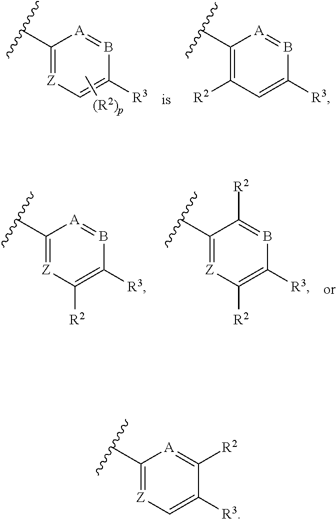

In a certain embodiment, when at least one selected from the group of A, B, and Z is N,

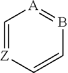

is a pyridine ring, a pyrimidine ring, or a pyridazine ring.

In a certain embodiment, when R¹ and R² are independently halogen, the halogen is fluorine, chlorine, bromine, or iodine, such as fluorine or chlorine.

In a certain embodiment, when R¹ and R² are independently C₁₋₆ alkyl, the C₁₋₆ alkyl is C₁₋₄ alkyl, such as methyl, ethyl, n-propyl, isopropyl, n-butyl, sec-butyl, isobutyl, or tert-butyl.

In a certain embodiment, when ring Y is a 3- to 6-membered cycloalkyl ring, the 3- to 6-membered cycloalkyl ring is a cyclopropyl, cyclobutyl, cyclopentyl, or cyclohexyl ring, such as a cyclopropyl or cyclobutyl ring.

In a certain embodiment, when ring Y is a 3- to 6-membered heterocycloalkyl ring, the 3- to 6-membered heterocycloalkyl ring is a 3- to 6-membered heterocycloalkyl ring containing one N heteroatom, such as a 4-membered heterocycloalkyl ring containing one N heteroatom, also such as

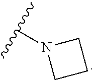 [SMILES: *N1CCC1]

In a certain embodiment, when R^{r} is independently C₁₋₆ alkyl, the C₁₋₆ alkyl is C₁₋₄ alkyl, such as methyl, ethyl, n-propyl, isopropyl, n-butyl, sec-butyl, isobutyl, or tert-butyl.

In a certain embodiment, when R^{r} is independently C₁₋₆ alkoxy, the C₁₋₆ alkoxy is C₁₋₄ alkoxy, such as methoxy, ethoxy, n-propoxy, isopropoxy, n-butoxy, sec-butoxy, isobutoxy, or tert-butoxy.

In a certain embodiment, when R⁵ and R⁶ are independently C₂₋₆ alkenyl, the C₂₋₆ alkenyl is C₂₋₃ alkenyl, such as vinyl, propenyl, or allyl.

In a certain embodiment, when R⁵ and R⁶ are independently C₂₋₆ alkynyl or C₂₋₆alkynyl substituted by one or more than one R⁵⁻¹, the C₂₋₆ alkynyl in the C₂₋₆ alkynyl and C₂₋₆ alkynyl substituted by one or more than one R⁵⁻¹ is C₂₋₃ alkynyl, such as ethynyl, propynyl, or propargyl, such as ethynyl.

In a certain embodiment, when R⁵ and R⁶ are independently 5- to 6-membered heteroaryl or 5- to 6-membered heteroaryl substituted by one or more than one R⁵⁻², the 5- to 6-membered heteroaryl in the 5- to 6-membered heteroaryl and the 5- to 6-membered heteroaryl substituted by one or more than one R⁵⁻² is 5- to 6-membered heteroaryl containing 1 to 2 N heteroatoms, such as 5-membered heteroaryl containing 2 N heteroatoms, also such as pyrazolyl, further such as

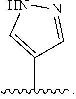 [SMILES: *c1cn[nH]c1]

In a certain embodiment, when R⁵⁻¹ and R⁵⁻² are independently halogen, the halogen is fluorine, chlorine, bromine, or iodine.

In a certain embodiment, when R⁵⁻¹ and R⁵⁻² are independently C₁₋₆ alkyl, the C₁₋₆ alkyl is C₁₋₄ alkyl, such as methyl, ethyl, n-propyl, isopropyl, n-butyl, sec-butyl, isobutyl, or tert-butyl, also such as methyl.

In a certain embodiment, when R⁵⁻¹ and R⁵⁻² are independently C₁₋₆ alkoxy, the C₁₋₆ alkoxy is C₁₋₄ alkoxy, such as methoxy, ethoxy, n-propoxy, isopropoxy, n-butoxy, sec-butoxy, isobutoxy, or tert-butoxy.

In a certain embodiment, when R³ is

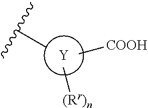

and ring Y is a 3 to 6-membered cycloalkyl ring, the

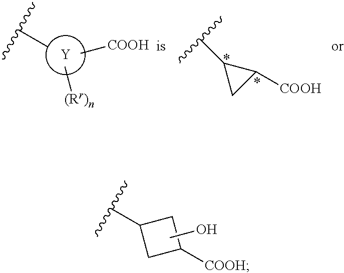

“*” means S configuration, R configuration, or a mixture of both.

In a certain embodiment, when is

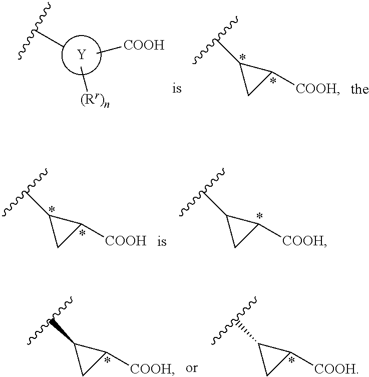

In a certain embodiment, when

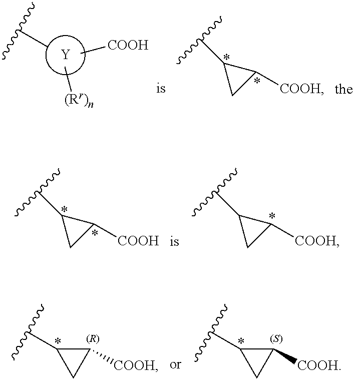

In a certain embodiment, when

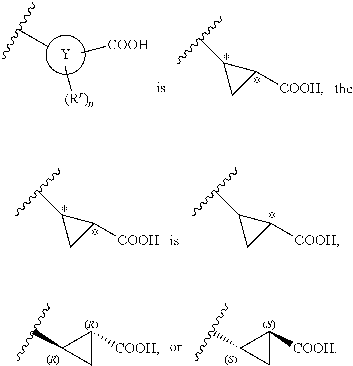

In a certain embodiment, when

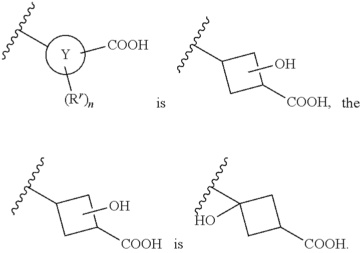

In a certain embodiment, when R³ is

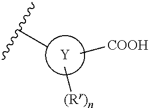

and ring Y is a 3- to 6-membered heterocycloalkyl ring, the

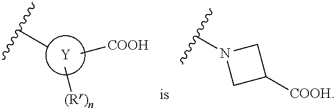

In a certain embodiment, when

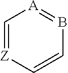

is a pyridine ring,

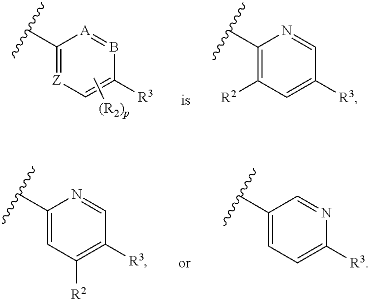

In a certain embodiment, when

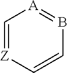

is a pyrimidine ring,

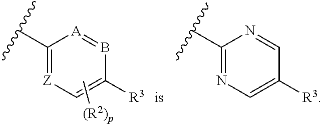

In a certain embodiment, when

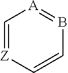

is a pyridazine ring,

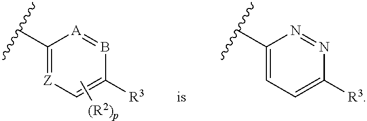

In a certain embodiment, when

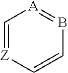

is a benzene ring,

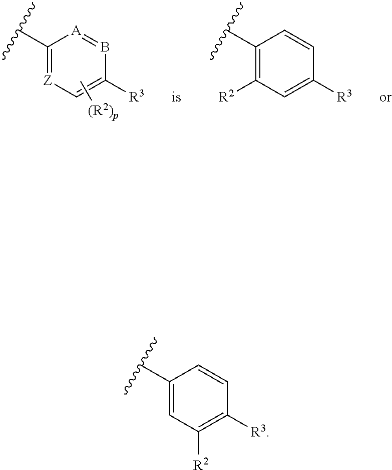

In a certain embodiment, when R¹ is fluorine and

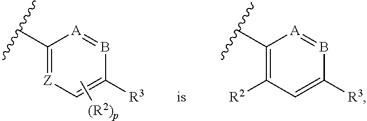

the R² is halogen.

In a certain embodiment, when R¹ is fluorine and

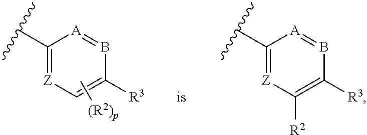

the R² is halogen or C₁₋₆ alkyl.

In a certain embodiment, when R¹ is chlorine and

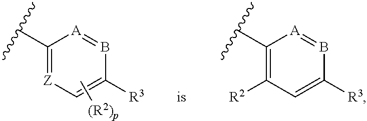

the R² is chlorine or C₁₋₆ alkyl.

In a certain embodiment, when R¹ is chlorine and

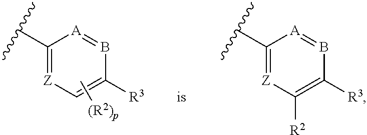

the R² is fluorine.

In a certain embodiment, when R¹ is H,

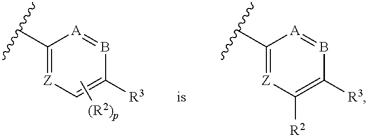

and R² is halogen or C₁₋₆ alkyl, the R² is chlorine or C₁₋₆ alkyl.

In a certain embodiment, when R¹ is H,

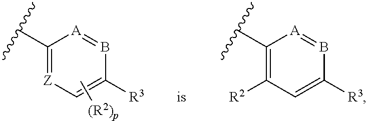

and R² is halogen or C₁₋₆ alkyl, the R² is halogen.

In a certain embodiment, when R¹ is chlorine and

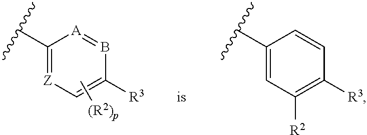

the R² is fluorine.

In a certain embodiment, when R¹ is chlorine and

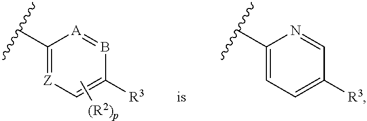

### the R³ is

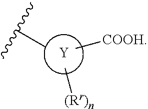

In a certain, embodiment, when R¹ is H,

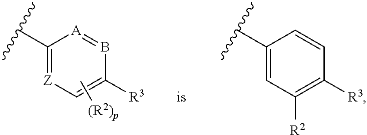

### and R³ is

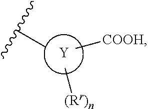

the R² is H, chlorine, or C₁₋₆ alkyl.

In a certain embodiment, when R¹ is H and

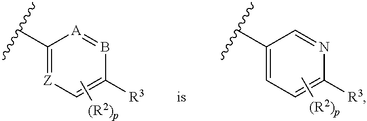

### the R³ is

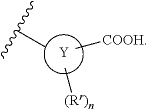

In a certain embodiment, when R¹ is H and

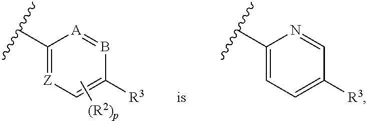

### the R³ is

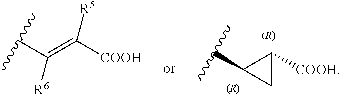

In a certain embodiment, when R¹ is H and

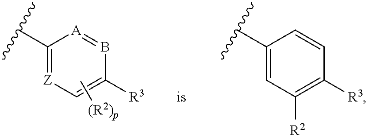

the R² is C₁₋₆ alkyl.

In a certain embodiment, when R¹ is H and

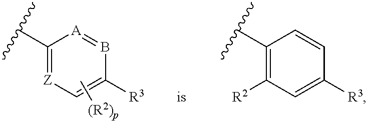

the R² is fluorine.

In a certain embodiment, when R¹ is fluorine or chlorine, and

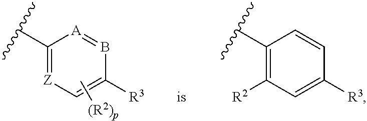

the R² is chlorine.

In a certain embodiment, when R¹ is chlorine or fluorine, and

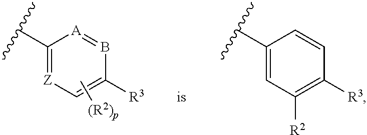

the R² is fluorine.

In a certain embodiment, when R¹ is H and

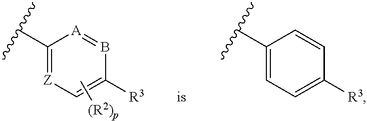

### the R³

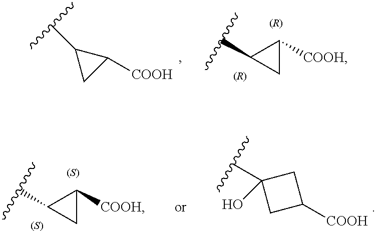

In a certain embodiment, when R₁ is H and

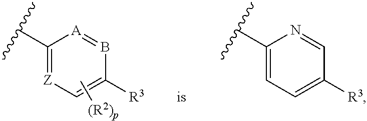

### the R³ is

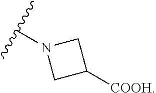 [SMILES: *N1CC(C(=O)O)C1]

In a certain embodiment, when R¹ is chlorine and R³ is

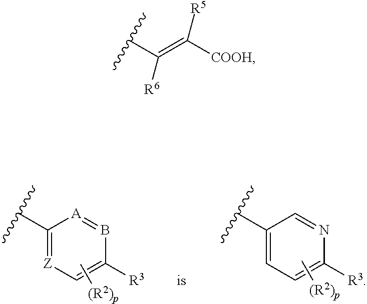

In a certain embodiment, when R¹ is chlorine and

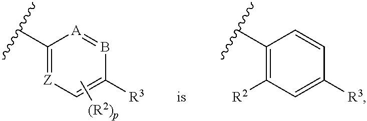

the R² is C₁₋₆ alkyl or halogen.

In a certain embodiment, when R¹ is fluorine and

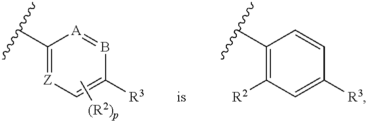

the R² is C₁₋₆ alkyl or chlorine.

In a certain embodiment, when R¹ is fluorine and

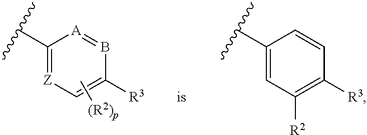

the R² is C₁₋₆ alkyl.

In a certain embodiment, when R₁ is H and

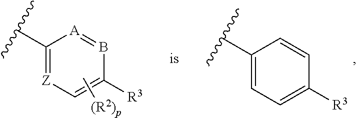

### the R³ is

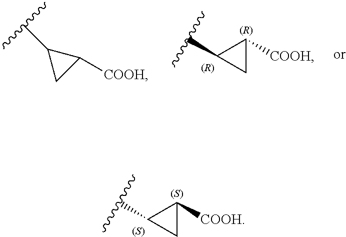

In a certain embodiment, when R¹ is H and

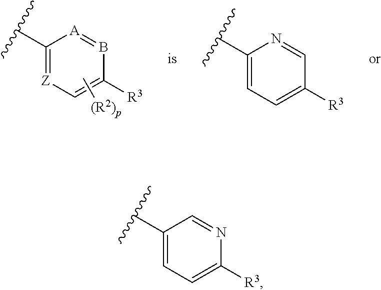

### the R³ is

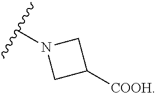 [SMILES: *N1CC(C(=O)O)C1]

In a certain embodiment,

in

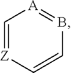

A, B, and Z are all CH, or at least one selected from the group of A, B, and Z is N;
R¹ is H or halogen;
the

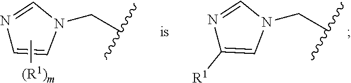

R² is H, halogen, or C₁₋₆ alkyl;
R³ is

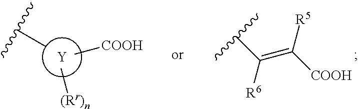

ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
R⁵ is independently H, C₂₋₆ alkynyl, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when A, B, and Z are CH at the same time, R⁵ and R⁶ are not H at the same time.

In a certain embodiment,

in

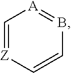

A, B, and Z are all CH, or at least one selected from the group of A, B, and Z is N;
R¹ is independently H or halogen;
the

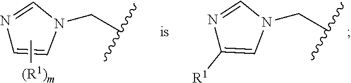

R² is H, halogen, or C₁₋₆ alkyl;

R³ is

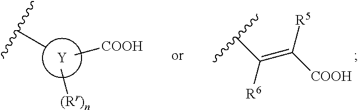

ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is

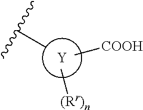

and ring Y is a 3- to 6-membered cycloalkyl ring, the

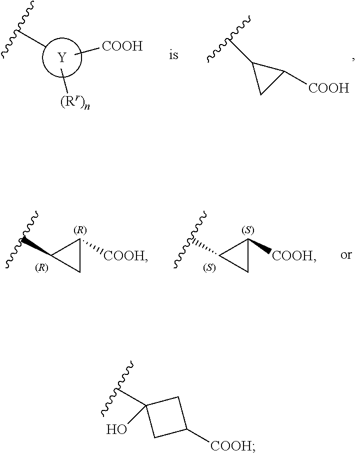

when R³ is

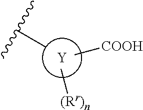

and ring Y is a 3- to 6-membered heterocycloalkyl ring, the

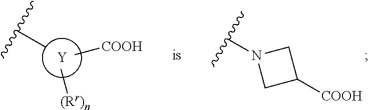

R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine and

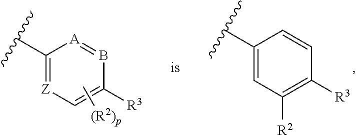

the R² is fluorine;
when R¹ is chlorine and

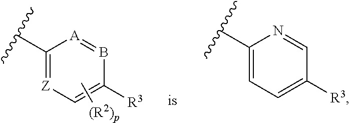

the R³ is

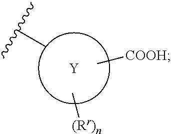

when R¹ is H,

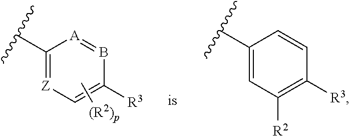

and R³ is

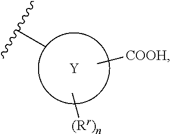

the R² is H, chlorine, or C₁₋₆ alkyl;
when R¹ is H and

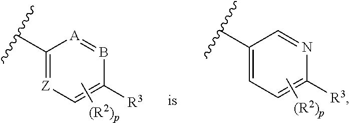

the R³ is

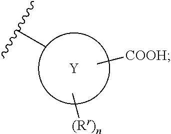

when R¹ is H and

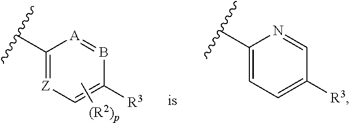

the R³ is

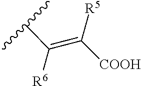

or

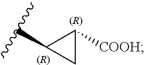

when A, B, and Z are CH at the same time, R⁵ and R⁶ are not H at the same time.

In a certain embodiment,

in

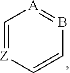

A, B, and Z are all CH, or at least one selected from the group of A, B, and Z is N;
R¹ is independently H or halogen;
the

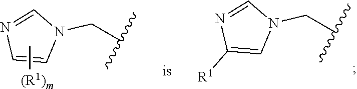

R² is H, halogen, or C₁₋₆ alkyl;
R³ is

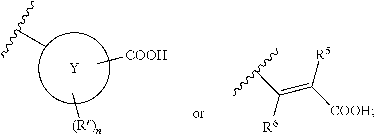

ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is

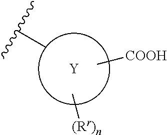

and ring Y is a 3- to 6-membered cycloalkyl ring, the

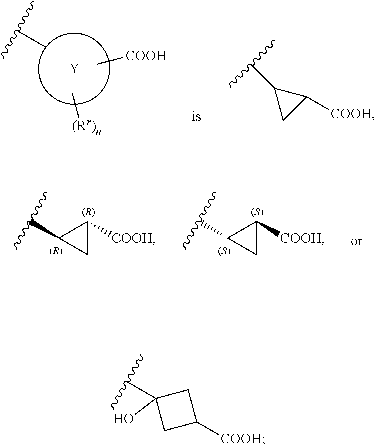

when R³ is

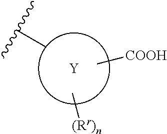

and ring Y is a 3- to 6-membered heterocycloalkyl ring, the

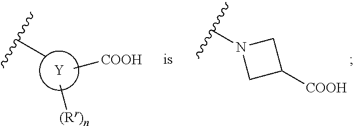

R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is H and

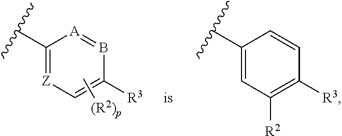

the R² is C₁₋₆ alkyl;
when R¹ is H and

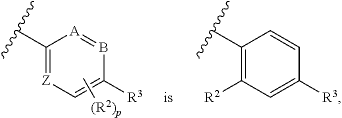

the R² is fluorine;
when R¹ is fluorine or chlorine, and

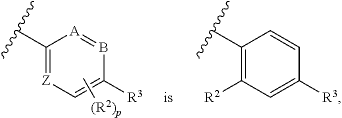

the R² is chlorine;
when R¹ is chlorine or fluorine, and

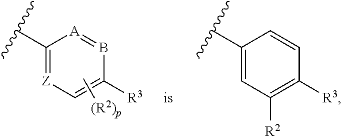

the R² is fluorine;
when R¹ is H and

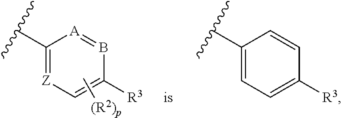

the R³ is

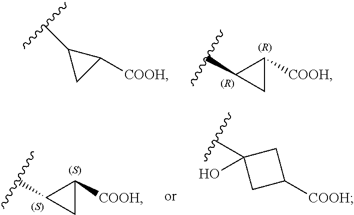

when R¹ is H and

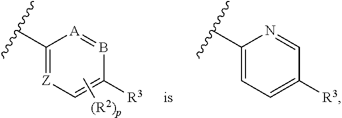

the R³ is

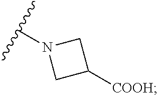 [SMILES: *N1CC(C(=O)O)C1]

when R¹ is chlorine and R³ is

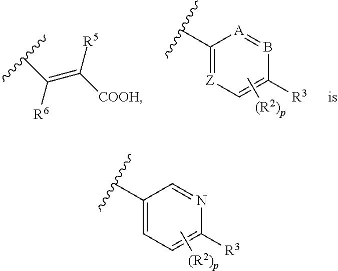

when A, B, and Z are CH at the same time, R⁵ and R⁶ are not H at the same time.

In a certain embodiment,
in

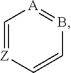

A, B, and Z are all CH, or at least one selected from the group of A, B, and Z is N;
R¹ is independently H or halogen;
the

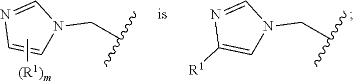

R² is H, halogen, or C₁₋₆ alkyl;
R³ is

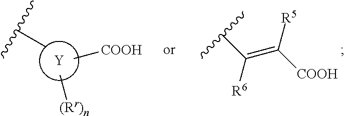

ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is

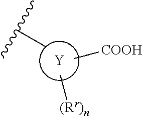

and ring Y is a 3- to 6-membered cycloalkyl ring, the

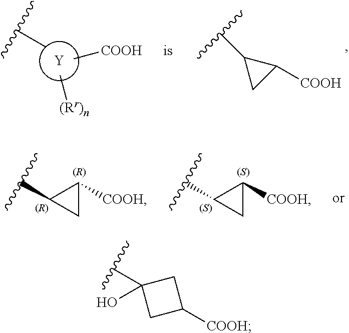

when R³ is

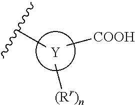

and ring Y is a 3- to 6-membered heterocycloalkyl ring, the

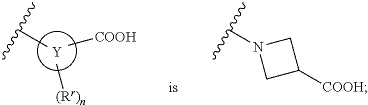

R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine and

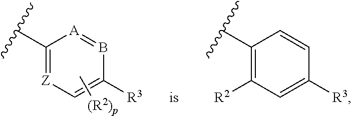

the R² is C₁₋₆ alkyl or halogen;
when R¹ is fluorine and

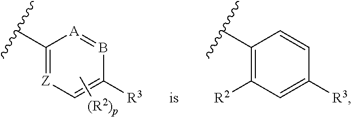

the R² is C₁₋₆ alkyl or chlorine;
when R¹ is fluorine and

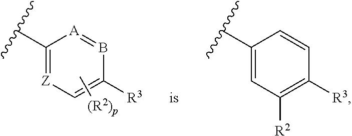

the R² is C₁₋₆ alkyl;
when R¹ is H

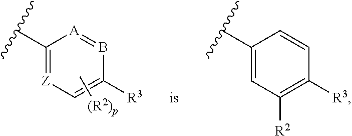

the R² is C₁₋₆ alkyl;
when R¹ is H and

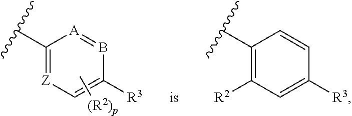

the R² is fluorine;
when R¹ is chlorine or fluorine, and

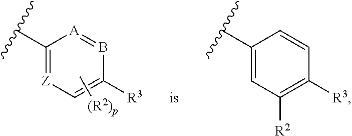

the R² is fluorine;
when R¹ is H and

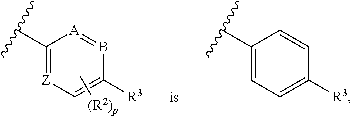

the R³ is

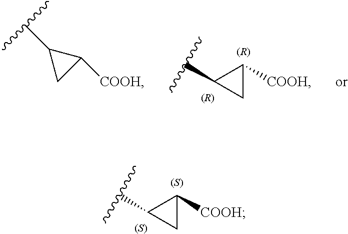

when R¹ is H and

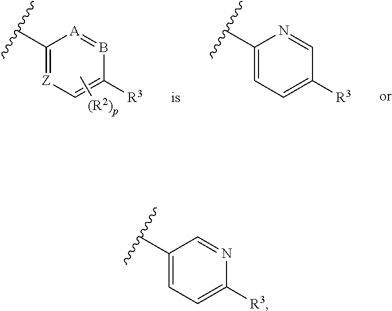

the R³ is

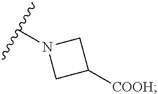 [SMILES: *N1CC(C(=O)O)C1]

when R¹ is chlorine and R³ is

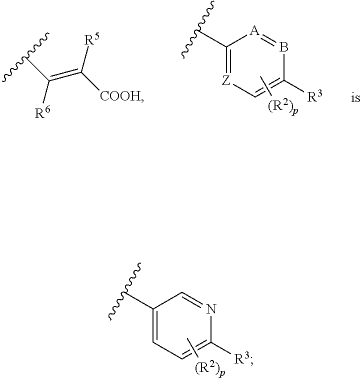

when A, B, and Z are CH at the same time, R⁵ and R⁶ are not H at the same time.

In a certain embodiment,
in

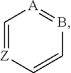

A, B, and Z are all CH, or at least one selected from the group of A, B, and Z is N;
R¹ is independently H or halogen;
the

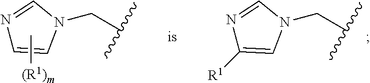

R² is H, halogen, or C₁₋₆ alkyl;
R³ is

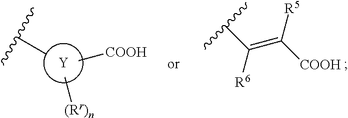

ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is

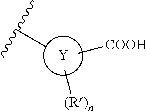

and ring Y is a 3- to 6-membered cycloalkyl ring, the

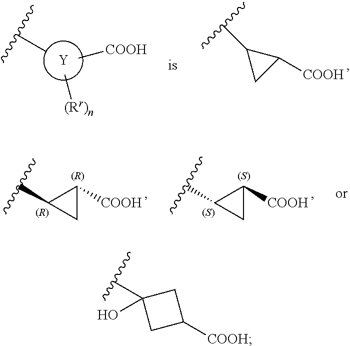

when R³ is

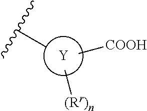

and ring Y is a 3- to 6-membered heterocycloalkyl ring, the

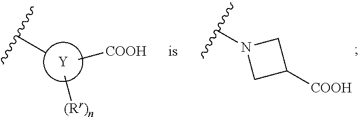

R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine or fluorine, and

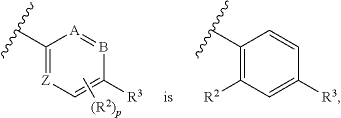

the R² is chlorine;
when R¹ is H,

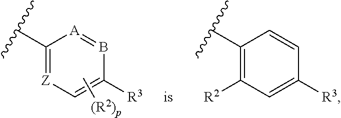

and R² is C₁₋₆ alkyl or halogen, the R² is fluorine;
when R¹ is H and

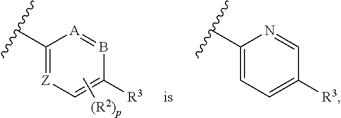

the R³ is

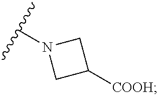 [SMILES: *N1CC(C(=O)O)C1]

when A, B, and Z are CH at the same time, R⁵ and R⁶ are not H at the same time.

In a certain embodiment,

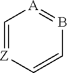

is a benzene ring;
R¹ and R² are independently H, chlorine, or fluorine;
the

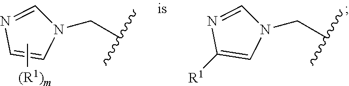

m and p are independently 0 or 1;
R³ is

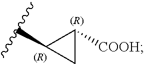

when R¹ is chlorine or fluorine, and

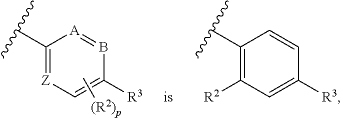

the R² is chlorine;
when R¹ is chlorine or fluorine, and

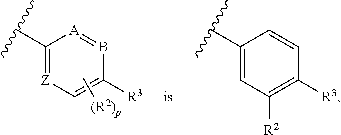

the R² is fluorine;
when R¹ is H and

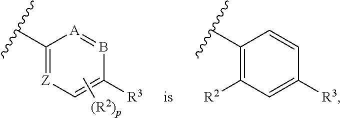

the R² is H or fluorine.

In a certain embodiment,

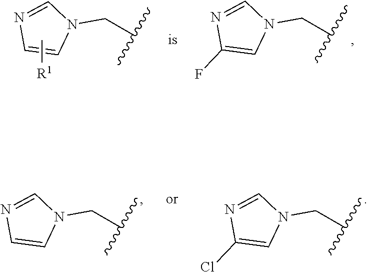

In a certain embodiment,

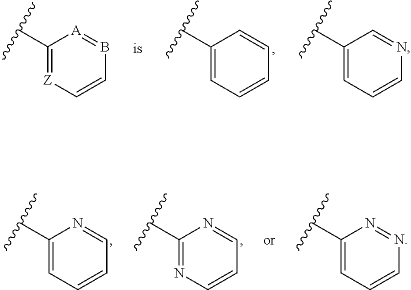

In a certain embodiment, when R³ is

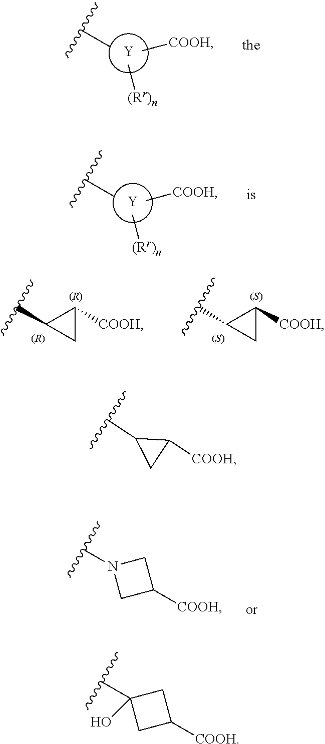

In a certain embodiment, when R³ is

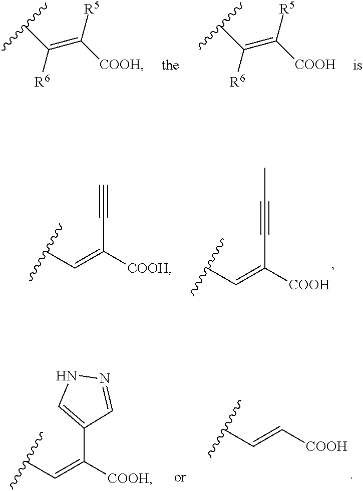

In a certain embodiment,

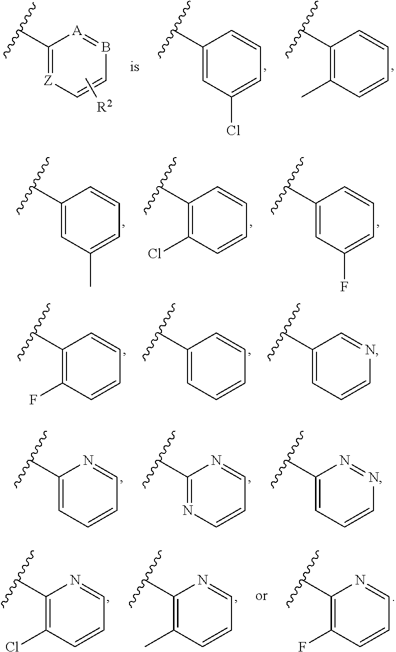

In a certain embodiment,

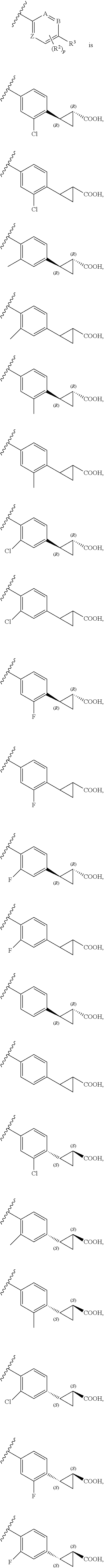

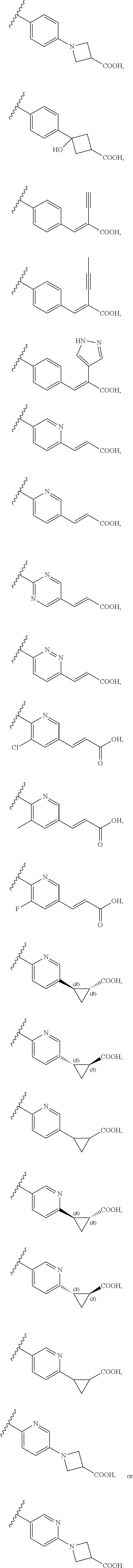

In a certain embodiment, the imidazole compound of formula I has any one of the following structures:

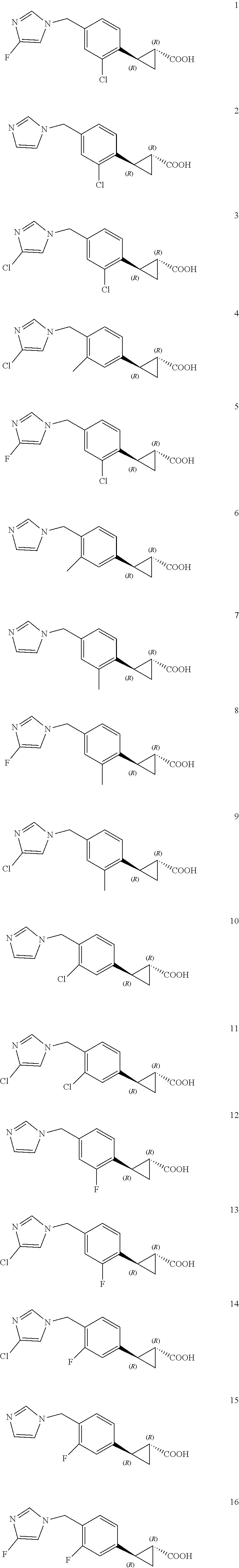

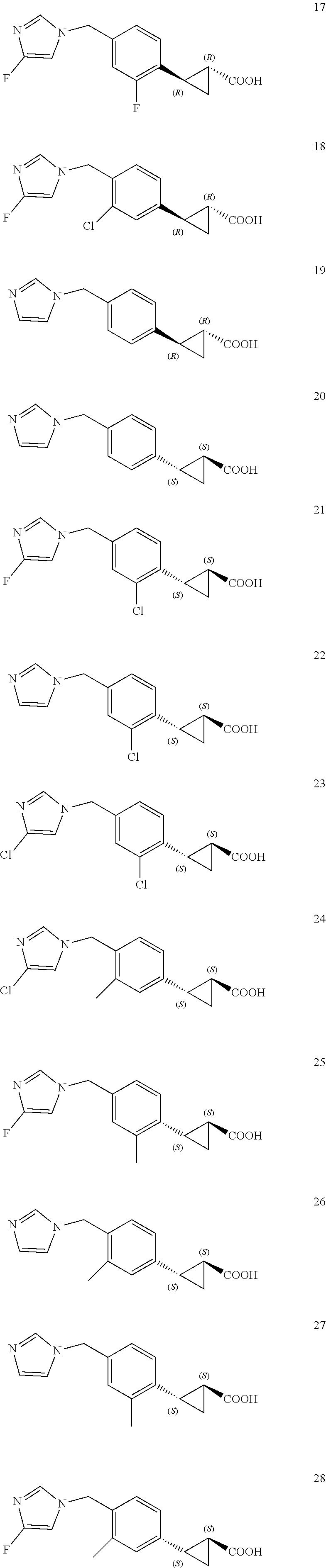

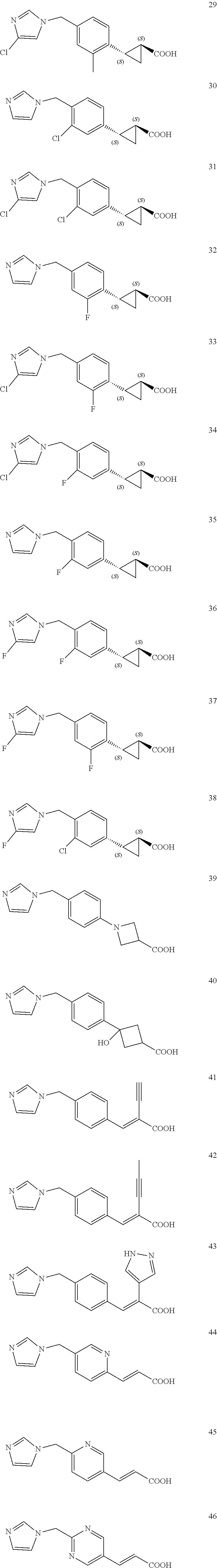

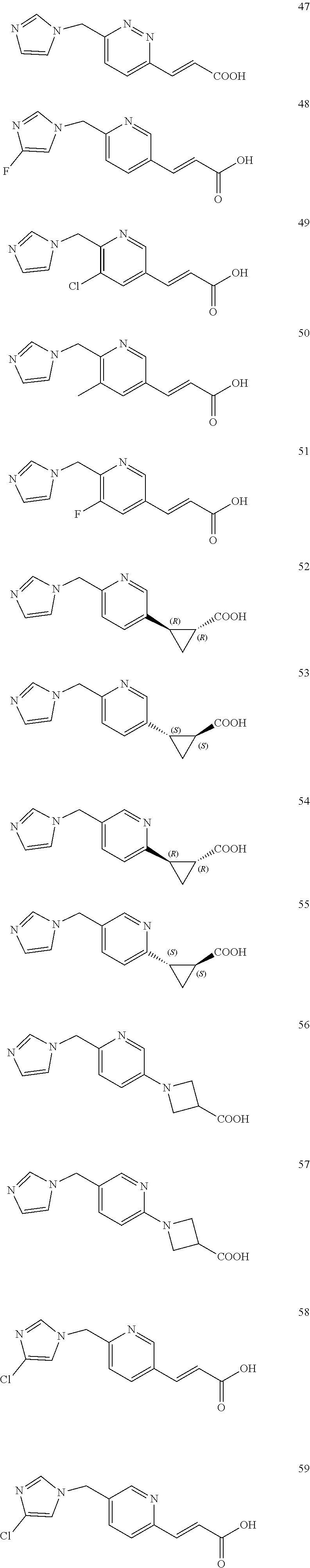

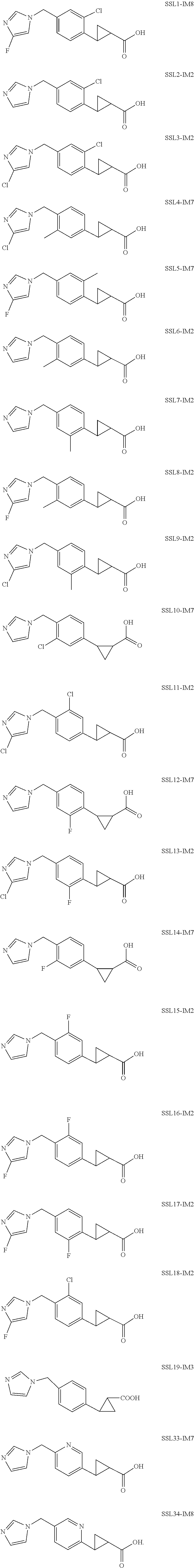

The present disclosure also provides a compound of formula II or III,

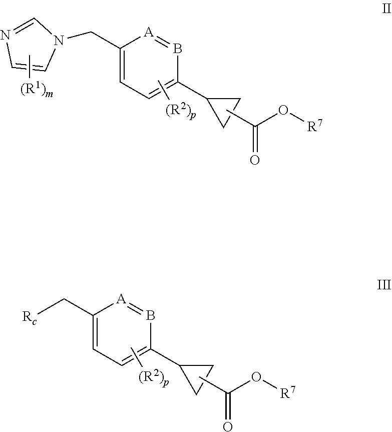

wherein A, B, m, p, R¹, and R² are defined as above;
R_{c} is —OH, a leaving group (e.g., Cl or Br), or an —O-hydroxyl protecting group (e.g., TBS or TBDMS);
R⁷ is C₁₋₆ alkyl; preferably, the C₁₋₆ alkyl is methyl, ethyl, n-propyl, isopropyl, n-butyl, sec-butyl, isobutyl, or tert-butyl, such as methyl or ethyl.

In a certain embodiment, the compound of formula II or III is any one of the following compounds:

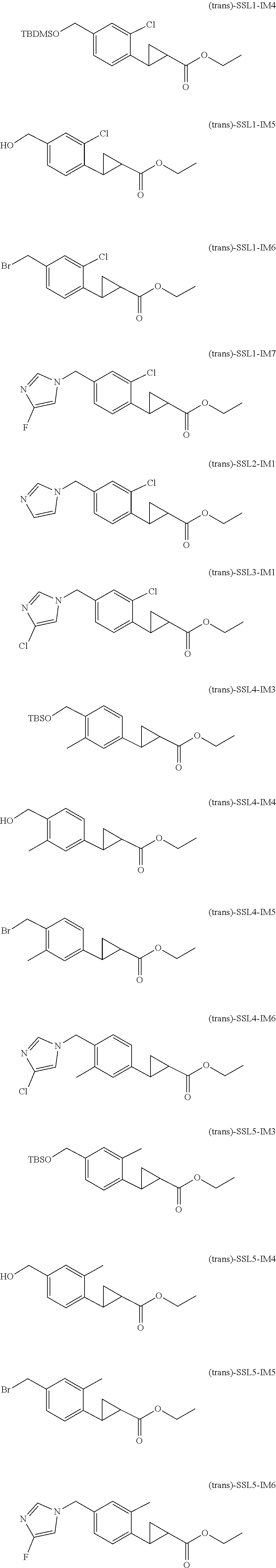

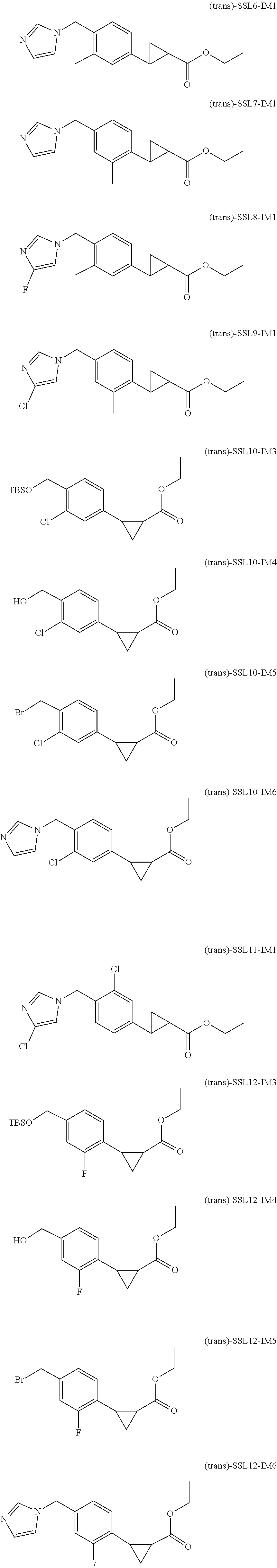

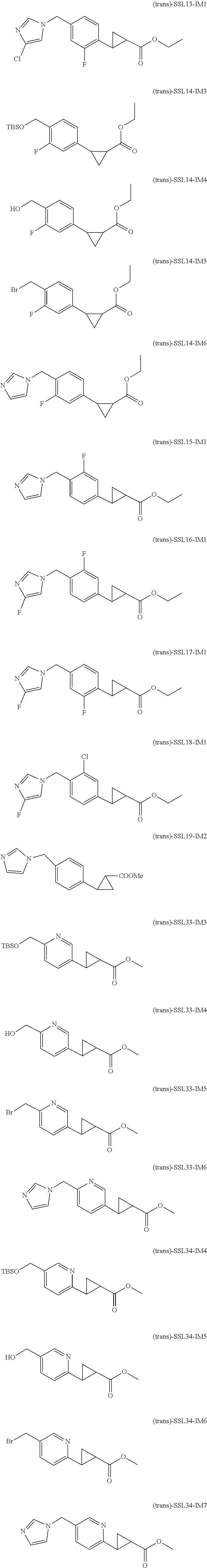

In the compound of formula II or III, “(trans)-” represents a trans configuration.

The present disclosure also provides a pharmaceutical composition comprising substance A and a pharmaceutical excipient; the substance A is a therapeutically effective amount of the imidazole compound of formula I or the pharmaceutically acceptable salt thereof.

The present disclosure also provides a use of substance A in the manufacture of a TXA₂ synthase inhibitor, the substance A is the imidazole compound of formula I or the pharmaceutically acceptable salt thereof.

In the use of the substance A in the manufacture of the TXA₂ synthase inhibitor, the TXA₂ synthase inhibitor can be used in mammalian organisms in vivo; it can also be used in vitro, mainly for experimental purposes, e.g., to provide a comparison as a standard sample or a control sample or to make a kit according to the conventional methods in the art, to provide a rapid assay for the inhibitory effect of platelet aggregation.

The present disclosure also provides a use of substance A in the manufacture of a medicament, the substance A is the imidazole compound of formula I or the pharmaceutically acceptable salt thereof, the medicament is used for the treatment and prevention of a TXA₂-related disease.

In the use of the substance A in the manufacture of the medicament, preferably, the disease related to TXA₂ is a thrombotic disease.

In the use of the substance A in the manufacture of the medicament, the thrombotic disease such as myocardial infarction, pulmonary embolism, or cerebral thrombosis.

The present disclosure also provides a use of substance A in the manufacture of a medicament, the medicament is used for the treatment and prevention of a thrombotic disease; the substance A is the imidazole compound of formula I or the pharmaceutically acceptable salt thereof.

In the use of the substance A in the manufacture of the medicament, the thrombotic disease such as myocardial infarction, pulmonary embolism, or cerebral thrombosis.

The present disclosure also provides a single crystal of a compound of formula A1, and the structure data of the single crystal are as follows:

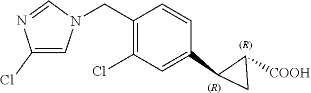

A1

| | Single crystal of the | |
|---|---|---|
| Parameters | compound of formula A1 | |
| Experimental formula | C14H12Cl2N2O2 | |
| Molecular weight | 311.16 | |
| Temperature | 113.15 | K |
| Wavelength | 0.71073 | A |
| Crystal system | Hexagonal | |
| Space group | P 61 | |
| Crystal cell size | a = 10.5982(3) Å, α = 90°. | |
| | b = 10.5982(3) Å, β= 90°. | |
| | c = 21.6750(8) Å, γ = 120°. | |
| Crystal cell volume | 2108.40(14) Å³ | |
| Z | 6 | |
| Density (calculated) | 1.470 | Mg/m³ |
| Absorption coefficient | 0.463 | mm⁻¹ |

| | |
|---|---|
| F(000) | 960.0 |
| Crystal size | 0.21 × 0.19 × 0.15 mm³ |
| θ range for data collection | 4.438 to 67.122° |
| Range of indicators | −16 ≤ h ≤ 16, −16 ≤ k ≤ 16, −29 ≤ l ≤ 32 |
| Diffraction point collection | 25979 |
| Independent diffraction points | 5172 [Rint = 0.0599, Rsigma = 0.0513] |
| Refinement method | Goodness-of-fit on F² |

The present disclosure also provides a single crystal of a compound of formula A2, and the structure data of the single crystal are as follows:

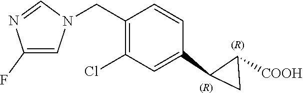

A2

| | Single crystal of the | |
|---|---|---|
| Parameters | compound of formula A2 | |
| Experimental formula | C14H12ClFN2O2 | |
| Molecular weight | 294.71 | |
| Temperature | 113.15 | K |
| Wavelength | 0.71073 | A |
| Crystal system | Monoclinic | |
| Space group | P 21 | |
| Crystal cell size | a = 4.5886(2) Å, α = 90°. | |
| | b = 10.9726(3) Å, β = 99.554(4)º. | |
| | c = 13.3970(5) Å, γ = 90°. | |
| Crystal cell volume | 665.17(4) Å³ | |
| Z | 2 | |
| Density (calculated) | 1.471 | Mg/m³ |
| Absorption coefficient | 0.301 | mm⁻¹ |

| | |
|---|---|
| F(000) | 304.0 |
| Crystal size | 0.23 × 0.2 × 0.17 mm |
| θ range for data collection | 4.826 to 65.816° |
| Range of indicators | −6 ≤ h ≤ 6, −16 ≤ k ≤ 16, −20 ≤ l ≤ 19 |
| Diffraction point collection | 9980 |
| Independent diffraction points | 4493 [Rint = 0.0310, Rsigma = 0.0400] |
| Refinement method | Goodness-of-fit on F² |

1 .

In the present disclosure, the imidazole compound of formula I can contain one or more than one chiral carbon atom, so that the imidazole compound can be separated into optically pure isomers, such as pure enantiomers, or racemates. Pure single isomers can be obtained by the separation methods in the art, such as chiral crystallization into salts, or separation by chiral preparative column.

In the present disclosure, if the imidazole compound of formula I or the pharmaceutically acceptable salt thereof has a stereoisomer, then they can present in the form of a single stereoisomer or their mixtures (such as racemates).

In addition to the foregoing, when used in the description and claims of the present disclosure, the following terms have the meanings shown below unless otherwise specified.

The term “stereoisomer” refers to a cis-trans isomer or an optical isomer. These stereoisomers can be separated, purified, and enriched by asymmetric synthesis methods or chiral separation methods (including but not limited to thin-layer chromatography, rotation chromatography, column chromatography, gas chromatography, high-pressure liquid chromatography, etc), and they can also be obtained by chiral separation through bonding with other chiral compounds (chemical bonding, etc.) or by forming salts (such as physical bonding). The term “single stereoisomer” means that the mass content of one stereoisomer of the compound of the present disclosure relative to all stereoisomers of the compound is not less than 95%.

In the present disclosure, when the carbon atom with “*” is a chiral carbon atom, the compound is S configuration, R configuration, or a mixture of both.

The term “halogen” refers to fluorine, chlorine, bromine, or iodine.

The term “alkyl” refers to a straight or branched alkyl group with a specified number of carbon atoms (e.g., C₁₋₆). The alkyl includes, but is not limited to, methyl, ethyl, n-propyl, isopropyl, n-butyl, tert-butyl, isobutyl, sec-butyl, n-pentyl, n-hexyl, n-heptyl, n-octyl, etc.

The term “alkoxy” refers to the group R_{X}—O—, wherein R_{X} is the alkyl as defined above.

The term “cycloalkyl” refers to a saturated monocyclic group consisting only of carbon atoms with a specified number of carbon atoms (e.g., 3- to 6-membered). The cycloalkyl includes, but is not limited to, cyclopropyl, cyclobutyl, cyclopentyl, cyclohexyl, etc.

The term “heterocycloalkyl” refers to a cyclic group with a specified number of ring atoms (e.g., 3- to 6-membered), a specified number of heteroatoms (e.g., 1, 2, or 3), and a specified type of heteroatom (one or more than one of N, O, and S), which is a monocyclic, bridged, or spiro ring, and which is saturated with respect to each ring. The heterocycloalkyl includes, but is not limited to, azetidinyl, tetrahydropyrrolyl, tetrahydrofuranyl, morpholinyl, piperidinyl, etc.

The term “alkenyl” refers to a straight or branched hydrocarbon chain group consisting only of carbon and hydrogen atoms, containing at least one double bond, having, for example, 2 to 14 (preferably 2 to 6, more preferably 2 to 4) carbon atoms, and is connected to the rest of the molecule by a single bond. Examples include but are not limited to vinyl, propenyl, allyl, but-1-enyl, but-2-enyl, pent-1-enyl, pent-1,4-dienyl, etc.

The term “alkynyl” refers to a straight or branched hydrocarbon chain group consisting only of carbon and hydrogen atoms, containing at least one triple bond and optionally one or more than one double bond, having, for example, 2 to 14 (preferably 2 to 6, more preferably 2 to 4) carbon atoms, and is connected to the rest of the molecule by a single bond. Examples include but are not limited to ethynyl, prop-1-ynyl, but-1-ynyl, pent-1-en-4-ynyl, etc.

The term “heteroaryl” refers to a cyclic group with a specified number of ring atoms (e.g., 5- to 6-membered), a specified number of heteroatoms (e.g., 1, 2, or 3), and a specified type of heteroatoms (one or more than one of N, O, and S), which is monocyclic or polycyclic, and at least one of the rings is aromatic (in accordance with Hückel's rule). For example, in one embodiment of the present disclosure, the 5- to 6-membered heteroaryl contains 1 to 4 heteroatoms selected from one or more than one of N, O, or S. The heteroaryl is connected to the other moiety of the molecule through rings that are aromatic or not. The heteroaryl includes, but is not limited to, furyl, pyrrolyl, thienyl, pyrazolyl, imidazolyl, oxazolyl, thiazolyl, pyridyl, pyrimidinyl, indolyl, pyridazinyl, etc.

The

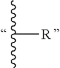

in the structural moiety means that the corresponding group R is connected to other moieties and groups in the compound through this site.

The term “pharmaceutically acceptable salt” refers to a salt obtained by reacting a compound with a pharmaceutically acceptable (relatively non-toxic, safe, and suitable for a patient) acid or base. When the compound contains a relatively acidic functional group, a base addition salt can be obtained by bringing the free form of the compound into contact with a sufficient amount of the pharmaceutically acceptable base in a suitable inert solvent. Pharmaceutically acceptable base addition salts include, but are not limited to, sodium salts, potassium salts, calcium salts, aluminum salts, magnesium salts, bismuth salts, ammonium salts, etc. When the compound contains a relatively basic functional group, an acid addition salt can be obtained by bringing the free form of the compound into contact with a sufficient amount of the pharmaceutically acceptable acid in a suitable inert solvent. Pharmaceutically acceptable acid addition salts include, but are not limited to, hydrochloride, sulfate, methanesulfonate, etc. For details, see Handbook of Pharmaceutical Salts: Properties, Selection, and Use (P. Heinrich Stahl, 2002).

The term “pharmaceutical excipient” or “pharmaceutically acceptable carrier” refers to the vehicles and additives used in the manufacture of drugs and the formulation of prescriptions, which are all substances contained in pharmaceutical preparations except active ingredients. Available in the Pharmacopoeia of the People's Republic of China (2015 Edition) Part IV, or, Handbook of Pharmaceutical Excipients (Raymond C Rowe, 2009 Sixth Edition). The excipient is mainly used to provide a safe, stable, and functional pharmaceutical composition, and can also provide a method for the subject to dissolve the active ingredient at a desired rate after administration, or to facilitate effective absorption of the active ingredient after the subject receives administration of the composition. The pharmaceutical excipient can be an inert filler or provide a certain function, such as stabilizing the overall pH value of the composition or preventing degradation of the active ingredient in the composition. The pharmaceutical excipient can include one or more than one of the following excipients: binders, suspending agents, emulsifiers, diluents, fillers, granulating agents, adhesives, disintegrants, lubricants, anti-adhesion agents, glidants, wetting agents, gelling agents, absorption delaying agents, dissolution inhibitors, enhancers, adsorbents, buffers, chelating agents, preservatives, colorants, flavorings, and sweeteners.

The pharmaceutical composition of the present disclosure can be prepared in accordance with the disclosure using any method known to those skilled in the art. For example, conventional mixing, dissolving, granulating, emulsifying, milling, encapsulating, embedding, or lyophilization processes.

The pharmaceutical composition of the present disclosure can be administered in any form, including injection (intravenous) administration, mucosal administration, oral (solid and liquid formulations) administration, inhaled administration, ocular administration, rectal administration, topical or parenteral (infusion, injection, implant, intravenous, subcutaneous, intravenous, intraarterial, intramuscular) administration. The pharmaceutical composition of the present disclosure can also be in a controlled-release or delayed-release dosage form (such as liposomes or microspheres). Examples of the solid oral dosage form include, but are not limited to, powders, capsules, caplets, softgels, and tablets. Examples of liquid formulations for oral or mucosal administration include, but are not limited to, suspensions, emulsions, elixirs, and solutions. Examples of topical formulations include, but are not limited to, emulsions, gels, ointments, creams, patches, pastes, foams, lotions, drops, or serum formulations. Examples of formulations for parenteral administration include, but are not limited to, solutions for injection, dry formulations which can be dissolved or suspended in a pharmaceutically acceptable carrier, suspensions for injection, and emulsions for injection. Examples of other suitable formulations of the pharmaceutical compositions include, but are not limited to, eye drops and other ophthalmic formulations; aerosols such as nasal sprays or inhalants; liquid dosage forms suitable for parenteral administration; suppositories and lozenge agents.

The term “treatment” refers to curative therapies or palliative measures. When referring to a specific disorder, treatment refers to: (1) ameliorating one or more biological manifestations of the disease or disorder, (2) interfering with (a) one or more points in the biological cascade leading to or causing the disorder or (b) one or more biological manifestations of the disorder, (3) ameliorating one or more symptoms, effects, or side effects associated with the disorder, or one or more symptoms, effects or side effects associated with the disorder or its treatment, or (4) slowing the progression of the disorder or one or more biological manifestations of the disorder. “Treatment” can also refer to prolonged survival compared to the expected survival without treatment.

The term “prevention” refers to the reduction of the risk of acquiring or developing diseases or disorders.

The term “therapeutically effective amount” refers to the amount of a compound that is sufficient to effectively treat the diseases or disorders described herein when administered to a patient. The “therapeutically effective amount” will vary according to the compound, the disease and its severity, and the age of the patient to be treated, but it can be adjusted by those skilled in the art as needed.

The above preferred conditions can be combined arbitrarily to obtain preferred embodiments of the present disclosure without violating common knowledge in the art.

The reagents and starting materials used in the present disclosure are all commercially available.

The positive progressive effect of the present disclosure is that an imidazole compound, an intermediate thereof, and a use thereof are provided. The imidazole compound of formula I of the present disclosure can significantly inhibit AA-induced platelet aggregation, improve MCAO/R-induced cerebral ischemic injury in rats, possess excellent metabolic stability, and improve the distribution of the drug in the brain tissue so as to enhance its pharmacodynamic activity for the treatment of acute thrombotic cerebral infarction and movement disorders accompanying cerebral infarction.

### BRIEF DESCRIPTION OF THE DRAWINGS

FIG. 1 is a diagram of a single crystal structure of compound 11 in example 11.

FIG. 2 is a diagram of a single crystal structure of compound 18 in example 18.

### DETAILED DESCRIPTION OF THE PREFERRED EMBODIMENT

The present disclosure is further illustrated below by means of examples, but the present disclosure is not limited to the scope of the examples. The experimental methods that haven't illustrated specific conditions in the following examples are selected according to conventional methods and conditions, or according to the product instructions.

### Example 1 Synthesis of Compound 1 and Compound 21

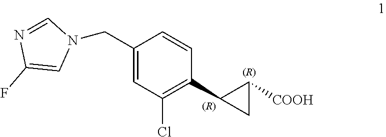

### (1R,2R)-2-(2-Chloro-4-((4-fluoro-1H-imidazol-1-yl)methyl)phenyl)cyclopropane-1-carboxylic acid

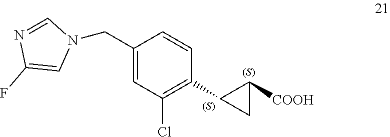

### (1S,2S)-2-(2-Chloro-4-((4-fluoro-1H-imidazol-1-yl)methyl)phenyl)cyclopropane-1-carboxylic acid

### Synthetic Route:

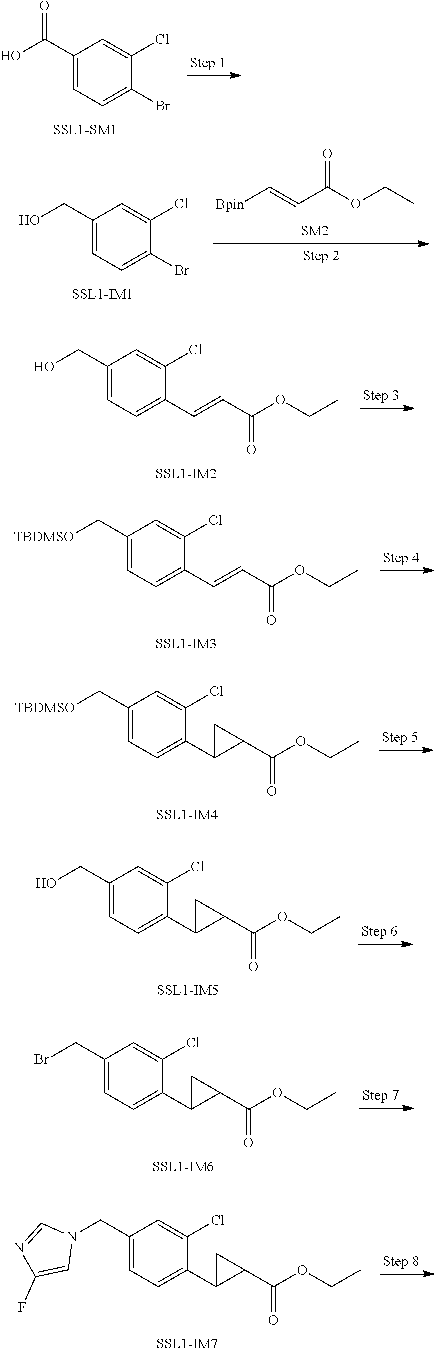

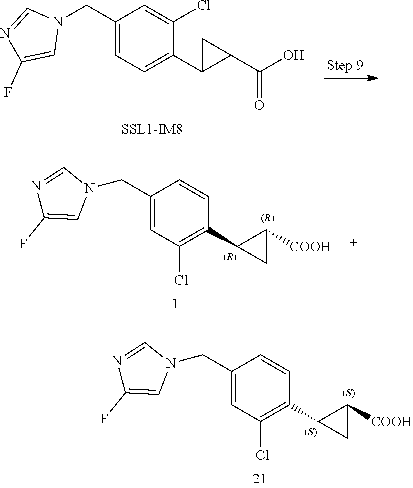

### Step 1

SSL1-SM1 (4.5 g, 19.08 mmol, 1.0 eq) and tetrahydrofuran (45 mL) were added to a 250 mL single-neck flask under N₂ atmosphere, stirred and dissolved, and a solution of borane-tetrahydrofuran (57.24 mL, 57.24 mmol, 1.0 M, 3.0 eq) was slowly added dropwise thereto at 0° C. and warmed to room temperature, and the reaction mixture was stirred and reacted for 3 hours. TLC (PE/EA 2:1) was used to monitor the reaction until the reaction was complete. The reaction was quenched by the dropwise addition of methanol in an ice bath, and the mixture was washed with saturated sodium bicarbonate solution and extracted with ethyl acetate. The phases were separated, and the organic phases were combined and dried. The organic phase was subjected to rotary evaporation until dryness to obtain the crude product, which was directly used in the next step.

### Step 2

SSL1-IM1 (about 4.2 g, 19 mmol, 1.0 eq), borate SM2 (6.4 g, 28.5 mmol, 1.5 eq), Pd(dppf)Cl₂ (1.39 g, 1.9 mmol, 0.1 eq), potassium carbonate (7.88 g, 57 mmol, 3.0 eq) were added to a 500 mL single-neck flask under N₂ atmosphere, and dioxane and water were added thereto, and then the reaction mixture was heated to 100° C. and stirred for 2 hours. The complete reaction of the raw material was monitored by TLC (PE/EA 2:1). After the reaction was complete, the reaction mixture was subjected to rotary evaporation until dryness, and the residue was subjected to column chromatography (PE/EA 2:1) to obtain about 4.6 g of a yellow liquid.

### Step 3

SSL1-IM2 was dissolved in DCM at room temperature, and imidazole (2.58 g, 38 mmol, 2.0 eq) was added thereto, and then TBSCl (3.43 g, 22.8 mmol, 1.2 eq) was added dropwise thereto; the reaction mixture was stirred for 3 hours after the addition was complete.

The complete reaction of the raw material was monitored by TLC (PE/EA 5:1). The solvent was removed by rotary evaporation until dryness, and the residue was subjected to column chromatography (PE/EA 20:1) to obtain 2.67 g of the product with a yield of 39% (the yield was the total yield of the three steps relative to the starting material).

### Step 4

Trimethylsulfoxonium iodide (0.55 g, 2.48 mmol, 1.1 eq), DMSO (6 mL), and NaH (0.1 g, 2.48 mmol, 1.1 eq) were added to a 50 mL single-neck flask under N₂ atmosphere. The reaction mixture was stirred for 1 hour at room temperature. Then all the prepared Ylide was added dropwise to a solution of SSL1-IM3 (0.8 g, 2.25 mmol, 1.0 eq) in DMSO (4 mL) and stirred at room temperature. The complete reaction of the raw material was monitored by TLC (PE/EA 20:1). After the reaction was complete, a small amount of water was added to quench the reaction, and the reaction mixture was extracted with ethyl acetate, washed three times with saturated brine, and dried. The organic phase was subjected to rotary evaporation until dryness, and the residue was subjected to column chromatography (PE/EA 20:1) to obtain the product (0.96 g, yield of 35%).

### Step 5

SSL1-IM4 (0.96 g, 2.6 mmol, 1.0 eq) was dissolved in THF, then TBAF (3.12 mL, 1.0 M, 3.12 mmol, 1.2 eq) was added thereto, and reaction mixture was stirred at room temperature for 2 hours. After the reaction was complete, the reaction mixture was subjected to rotary evaporation until dryness, and the residue was subjected to column chromatography (PE/EA 2:1) to obtain the product (0.54 g, yield of 82%).

### Step 6

SSL1-IM5 (0.54 g, 2.12 mmol, 1.0 eq) was dissolved in tetrahydrofuran, and then the mixture was added with triphenylphosphine (1.11 g, 4.24 mmol, 2.0 eq), cooled to 0° C., added with tetrabromomethane (1.05 g, 3.18 mmol, 1.5 eq) in batches, warmed to room temperature, and stirred for 2 hours. After the reaction was complete, the reaction mixture was subjected to rotary evaporation until dryness, and the residue was subjected to column chromatography (PE/EA 20:1) to obtain the product (0.352 g, yield of 52%).

### Step 7

SSL1-IM6 (0.116 g, 0.367 mmol, 1.0 eq), potassium carbonate (0.21 g, 1.468 mmol, 4.0 eq), and 5-fluoroimidazole (0.095 g, 1.101 mmol, 3.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (60 mg, yield of 55%).

### Step 8

SSL1-IM7 (0.06 g, 0.2 mmol, 1.0 eq) was dissolved in tetrahydrofuran and methanol, then the mixture was added dropwise with lithium hydroxide aqueous solution (0.02 g, 0.4 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (26 mg, yield of 45%). ¹H NMR (400 MHz, MeOD) δ 7.42 (t, J=1.4 Hz, 1H), 7.29 (d, J=1.7 Hz, 1H), 7.12 (dd, J=8.0, 1.7 Hz, 1H), 7.04 (d, J=8.0 Hz, 1H), 6.70 (dd, J=7.9, 1.7 Hz, 1H), 5.10 (s, 2H), 2.61 (ddd, J=8.9, 6.2, 4.5 Hz, 1H), 1.67 (dt, J=8.5, 5.3 Hz, 1H), 1.46 (ddd, J=9.3, 5.3, 4.2 Hz, 1H), 1.13 (ddd, J=8.4, 6.2, 4.2 Hz, 1H). Mass: [M+H]⁺ 295.0.

### Step 9

SSL1-IM8 was obtained and purified by a chiral preparative column to obtain compound land compound 21.

### Example 2 Synthesis of Compound 2 and Compound 22

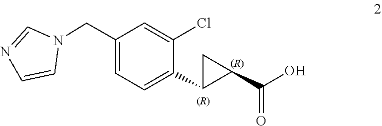

### (1R,2R)-2-(4-((1H-Imidazol-1-yl)methyl)-2-chlorophenyl)cyclopropane-1-carboxylic acid

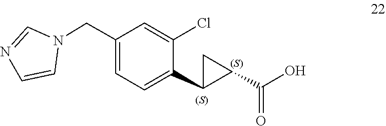

### (1S,2S)-2-(4-((1H-Imidazol-1-yl)methyl)-2-chlorophenyl)cyclopropane-1-carboxylic acid

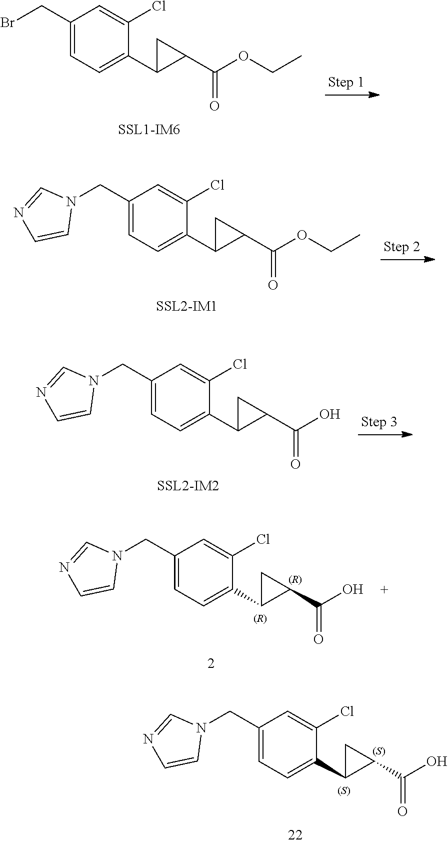

### Step 1

SSL1-IM6 (0.116 g, 0.367 mmol, 1.0 eq), potassium carbonate (0.21 g, 1.468 mmol, 4.0 eq), and imidazole (0.075 g, 1.101 mmol, 3.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (75 mg, yield of 67%).

### Step 2

SSL2-IM1 (0.075 g, 0.25 mmol, 1.0 eq) was dissolved in tetrahydrofuran and methanol, then the mixture was added dropwise with lithium hydroxide aqueous solution (0.021 g, 0.5 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (30 mg, yield of 45%). ¹H NMR (400 MHz, MeOD) δ 7.76 (s, 1H), 7.25 (d, J=1.6 Hz, 1H), 7.09 (dd, J=9.5, 2.9 Hz, 2H), 7.06-6.95 (m, 2H), 5.18 (s, 2H), 2.61 (ddd, J=9.0, 6.2, 4.6 Hz, 1H), 1.67 (dt, J=8.5, 5.1 Hz, 1H), 1.46 (ddd, J=9.3, 5.2, 4.3 Hz, 1H), 1.14 (ddd, J=8.4, 6.3, 4.2 Hz, 1H). Mass: [M+H]⁺ 277.0.

### Step 3

SSL2-IM2 was obtained and purified by a chiral preparative column to obtain compound 2 and Compound 22.

### Example 3 Synthesis of Compound 3 and Compound 23

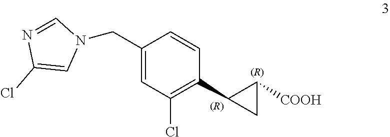

### (1R,2R)-2-(2-Chloro-4-((4-chloro-1H-imidazol-1-yl)methyl)phenyl)cyclopropane-1-carboxylic acid

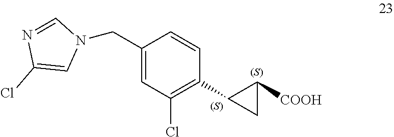

### (1S,2S)-2-(2-Chloro-4-((4-chloro-1H-imidazol-1-yl)methyl)phenyl)cyclopropane-1-carboxylic acid

### Synthetic Route

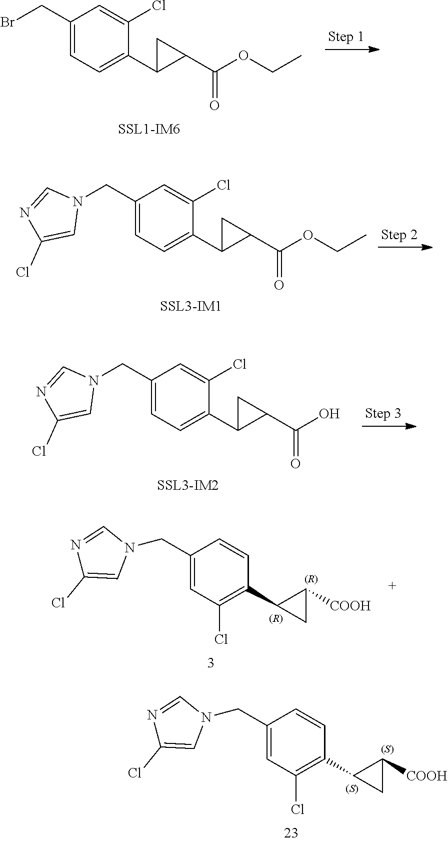

### Step 1

SSL1-IM6 (0.116 g, 0.367 mmol, 1.0 eq), potassium carbonate (0.21 g, 1.468 mmol, 4.0 eq), and 5-chloroimidazole (0.13 g, 1.101 mmol, 3.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (65 mg, yield of 55%).

### Step 2

SSL3-IM1 (0.065 g, 0.2 mmol, 1.0 eq) was dissolved in tetrahydrofuran and methanol, then the mixture was added dropwise with lithium hydroxide aqueous solution (0.021 g, 0.5 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (44 mg, yield of 71%). ¹H NMR (400 MHz, MeOD) δ 7.76 (s, 1H), 7.25 (d, J=1.6 Hz, 1H), 7.09 (dd, J=9.5, 2.9 Hz, 2H), 7.06-6.95 (m, 2H), 5.18 (s, 2H), 2.61 (ddd, J=9.0, 6.2, 4.6 Hz, 1H), 1.67 (dt, J=8.5, 5.1 Hz, 1H), 1.46 (ddd, J=9.3, 5.2, 4.3 Hz, 1H), 1.14 (ddd, J=8.4, 6.3, 4.2 Hz, 1H). Mass: [M+H]⁺ 311.0.

### Step 3

SSL3-IM2 was obtained and purified by a chiral preparative column to obtain compound 3 and Compound 23.

### Example 4 Synthesis of Compound 4 and Compound 24

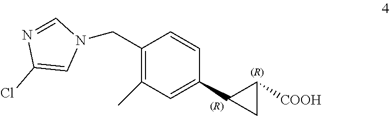

### (1R,2R)-2-(4-((4-Chloro-1H-imidazol-1-yl)methyl)-3-methylphenyl)cyclopropane-1-carboxylic acid

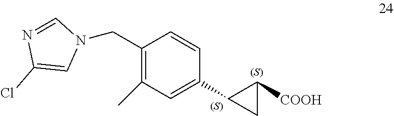

### (1S,2S)-2-(4-((4-Chloro-1H-imidazol-1-yl)methyl)-3-methylphenyl)cyclopropane-1-carboxylic acid

### Synthetic Route

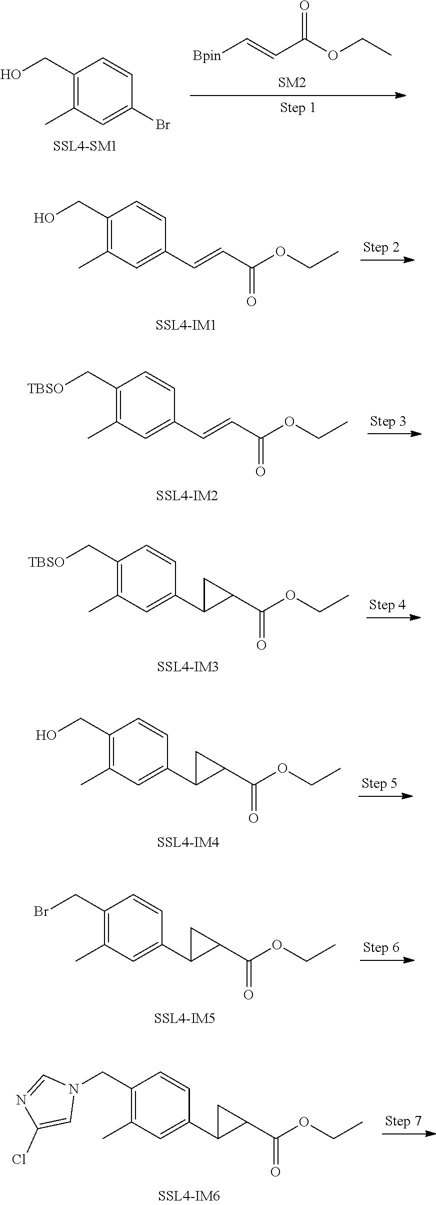

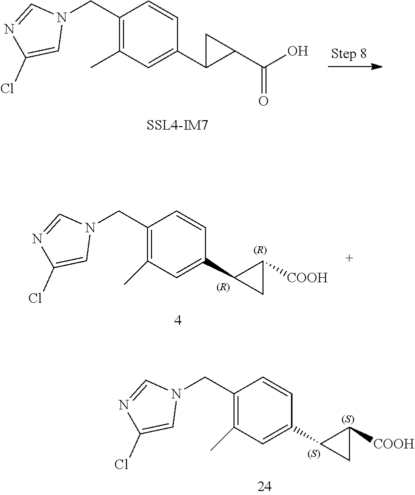

### Step 1

SSL4-SM1 (1.0 g, 4.97 mmol, 1.0 eq), borate SM2 (1.24 g, 5.47 mmol, 1.1 eq), Pd(dppf)Cl₂ dichloromethane complex (180 mg, 0.025 mmol, 0.05 eq), and cesium carbonate (3.24 g, 9.94 mmol, 2.0 eq) were added to a 100 mL single-neck flask under N₂ atmosphere, and dioxane and water were added thereto, and then the reaction mixture was heated to 100° C. and stirred overnight. The complete reaction of the raw material was monitored by TLC (PE/EA 3:1). After the reaction was complete, the reaction mixture was subjected to rotary evaporation until dryness, and the residue was subjected to column chromatography (PE/EA 4:1) to obtain the product (780 mg, yield of 72%).

### Step 2

SSL4-IM1 (780 mg, 3.54 mmol, 1.0 eq) was taken and dissolved in 25.0 mL of CH₂Cl₂, and imidazole (482 mg, 7.08 mmol, 2.0 eq) and TBSCl (641 mg, 4.25 mmol, 1.2 eq) were added thereto, and the reaction mixture was reacted at room temperature for 3.0 hours. TLC (PE/EA 5:1) was used to monitor the reaction until the starting material disappeared completely. The reaction mixture was washed with water and extracted with DCM, and the organic phases were combined, dried, concentrated, and then purified by column chromatography (PE:EA=10:1) to obtain the solid product (890 mg, yield of 75%).

### Step 3

10.0 mL of DMSO and NaH (160 mg, 3.99 mmol, 1.5 eq) were added to a 50 mL reaction flask, and 5 minutes after the addition, trimethylsulfoxonium iodide (880 mg, 3.99 mmol, 1.5 eq) was added thereto, and the reaction mixture was stirred at room temperature for 1.0 hour to obtain a clear and transparent solution. SSL4-IM2 (890 mg, 2.66 mmol, 1.0 eq) was added to another 25 mL reaction flask, dissolved in 3.0 mL of DMSO, which was added to the reaction mixture and washed twice with 1.0 mL of DMSO. The reaction mixture was reacted for 3.0 hours at room temperature, and TLC (PE/EA=10:1) was used to monitor the reaction until the starting material disappeared completely. The reaction mixture was washed with saturated NaCl, extracted with EA, dried, concentrated, and used directly in the next step.

### Step 4

The crude product of SSL4-IM3 (2.66 mmol, 1.0 eq) was added to a 50 mL reaction flask, dissolved in 15 mL of THF, and 1.0 M TBAF (3.2 mL, 3.19 mmol, 1.2 eq) was added thereto, and then the reaction mixture was reacted at room temperature for 1.0 hour. TLC (PE:EA=4:1) was used to monitor the reaction until the starting material disappeared completely. The reaction mixture was concentrated and purified by column chromatography (PE:EA=4:1) to obtain the product (140 mg, a two-step yield of 22%).

### Step 5

SSL4-IM4 (140 mg, 0.10 mmol, 1.0 eq) was taken and dissolved in 5.0 mL of CH₂Cl₂, placed in an ice bath, and CBr₄ (258 mg, 0.77 mmol, 1.3 eq) and PPh₃ (200 mg, 0.77 mmol, 1.3 eq) were added thereto, and then the reaction mixture was reacted for 20 minutes at this temperature. TLC (PE:EA=10:1) was used to monitor the reaction until the starting material disappeared completely. The reaction mixture was concentrated and purified by column chromatography (PE:EA=10:1) to obtain the product (160 mg, yield of 89%).

### Step 6

The product was dissolved in 2.0 mL of THF, added to a reaction flask, and 5-chloroimidazole (52 mg, 0.50 mmol, 2.5 eq) and NaH (20 mg, 0.50 mmol, 2.5 eq) were added thereto, reacted for 0.5 hours at room temperature, added with a solution of SSL4-IM5 (60 mg, 0.20 mmol, 1.0 eq) dissolved in 1.0 mL of THF, washed twice with 0.5 mL of THF, and the reaction mixture was reacted overnight at room temperature. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (27 mg, a two-step yield of 45%).

### Step 7

Compound SSL4-IM6 (27 mg, 0.085 mmol, 1.0 eq) was taken and dissolved in 0.8 mL of THF, and 0.4 mL of MeOH was added thereto. LiOH (11 mg, 0.254 mmol, 2.0 eq) was added to another EP tube, dissolved in 0.4 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=10:1) to obtain the product (17 mg, yield of 65%). 1H NMR (400 MHz, Methanol-d4) δ 7.54 (d, J=1.6 Hz, 1H), 7.05-7.00 (m, 2H), 6.97 (dd, J=7.7, 1.8 Hz, 2H), 5.16 (s, 2H), 2.43 (ddd, J=9.2, 6.5, 4.1 Hz, 1H), 2.24 (s, 3H), 1.83 (ddd, J=8.4, 5.3, 4.1 Hz, 1H), 1.52 (ddd, J=9.5, 5.3, 4.4 Hz, 1H), 1.35 (dt, J=6.5, 2.1 Hz, 1H). Mass: [M+H]⁺ 291.1.

### Step 8

SSL4-IM7 was obtained and purified by a chiral preparative column to obtain compound 4 and Compound 24.

### Example 5 Synthesis of Compound 5 and Compound 25

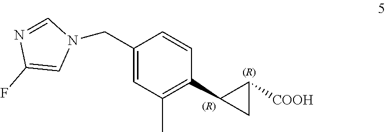

### (1R,2R)-2-(4-((4-Fluoro-1H-imidazol-1-yl)methyl)-2-methylphenyl)cyclopropane-1-carboxylic acid

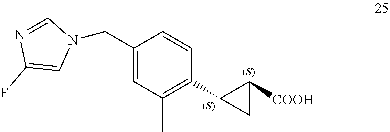

### (1S,2S)-2-(4-((4-Fluoro-1H-imidazol-1-yl)methyl)-2-methylphenyl)cyclopropane-1-carboxylic acid

### Synthetic Route

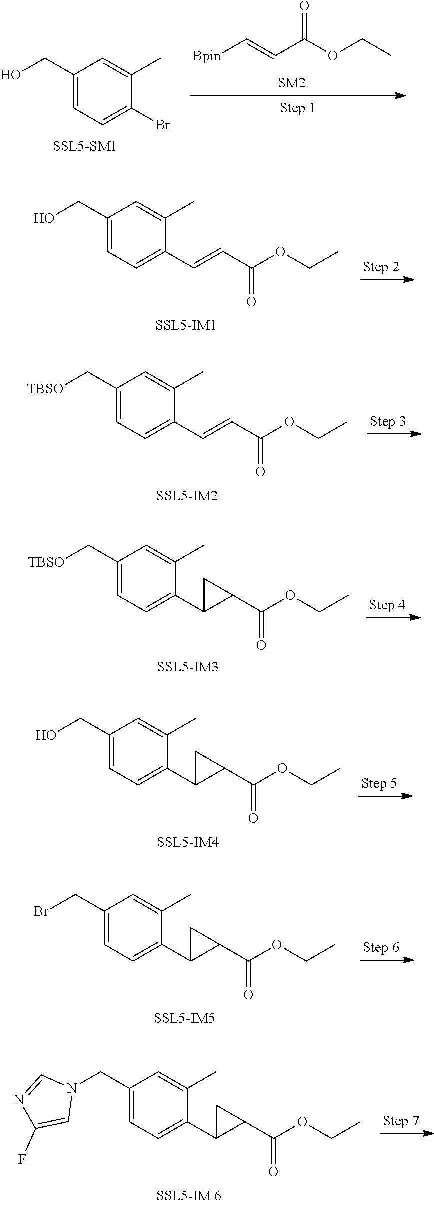

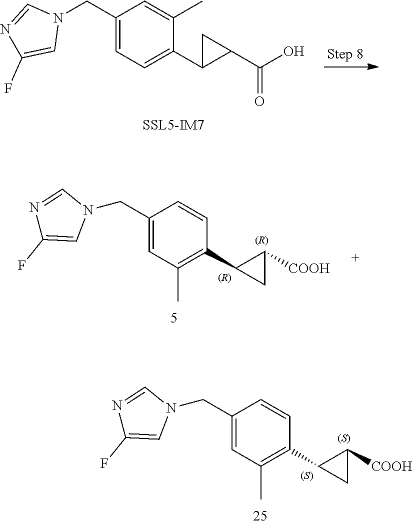

### Step 1

SSL5-SM1 (1.3 g, 6.7 mmol, 1.0 eq), borate SM2 (1.7 g, 7.4 mmol, 1.1 eq), Pd(dppf)Cl₂ dichloromethane complex (246 mg, 0.34 mmol, 0.05 eq), and cesium carbonate (4.4 g, 13.5 mmol, 2.0 eq) were added to a 100 mL single-neck flask under N₂ atmosphere, and dioxane and water were added thereto, and then the reaction mixture was heated to 100° C. and stirred for 10 hours. The complete reaction of the raw material was monitored by TLC (PE/EA 3:1). After the reaction was complete, the reaction mixture was subjected to rotary evaporation until dryness, and the residue was subjected to column chromatography (PE/EA 4:1) to obtain the product (1.5 g, yield of 99%).

### Step 2

SSL5-IM1 (1.5 g, 6.7 mmol, 1.0 eq) was taken and dissolved in 40 mL of CH₂Cl₂, and imidazole (930 mg, 13.6 mmol, 2.0 eq) and TBSCl (1.4 g, 8.9 mmol, 1.3 eq) were added thereto, and then the reaction mixture was reacted at room temperature for 5.0 hours. TLC (PE/EA 3:1) was used to monitor the reaction until the starting material disappeared completely. The reaction mixture was washed with water and extracted with DCM, and the organic phases were combined, dried, concentrated, and then purified by column chromatography (PE:EA=10:1) to obtain the solid product (1.7 g, yield of 76%).

### Step 3

30 mL of DMSO and NaH (264 mg, 6.61 mmol, 1.3 eq) were added to a 50 mL reaction flask, and 5 minutes after the addition, trimethylsulfoxonium iodide (145 mg, 6.61 mmol, 1.3 eq) was added thereto, and the reaction mixture was stirred at room temperature for 1.0 hour to obtain a clear and transparent solution. SSL5-IM2 (1.7 g, 5.08 mmol, 1.0 eq) was added to another 25 mL reaction flask, dissolved in 6.0 mL of DMSO, which was added to the reaction mixture and washed twice with 2.0 mL of DMSO. The reaction mixture was reacted for 4.0 hours, and TLC (PE/EA=20:1) was used to monitor the reaction until the starting material disappeared completely. The reaction mixture was washed with saturated NaCl solution, extracted with EA, concentrated, and used directly in the next step.

### Step 4

SSL5-IM3 (5.08 mmol, 1.0 eq) was added to a 50 mL reaction flask, dissolved in 20 mL of THF, and 1.0 M TBAF (4.0 mL, 4.0 mmol, 0.8 eq) was added thereto, and then the reaction mixture was reacted at room temperature for 4.0 hours. TLC (PE:EA=10:1) was used to monitor the reaction until the starting material disappeared completely. The reaction mixture was concentrated and purified by column chromatography (PE:EA=4:1) to obtain the product (268 mg, a two-step yield of 23%).

### Step 5

SSL5-IM4 (268 mg, 1.15 mmol, 1.0 eq) was taken and dissolved in 11 mL of CH₂Cl₂, placed in an ice bath, and CBr₄ (570 mg, 1.72 mmol, 1.5 eq) and PPh₃ (451 mg, 1.72 mmol, 1.5 eq) were added thereto, and then the reaction mixture was reacted for 2 hours at room temperature. TLC (PE:EA=3:1) was used to monitor the reaction until the starting material disappeared completely. The reaction mixture was concentrated and purified by column chromatography (PE:EA=20:1) to obtain the product (270 mg, yield of 79%).

### Step 6

The product was dissolved in 2.0 mL of THF, added to a reaction flask, and 5-fluoroimidazole (78 mg, 0.93 mmol, 3.0 eq) and NaH (36 mg, 0.93 mmol, 3.0 eq) were added thereto, reacted for 0.5 hours at room temperature, added with a solution of SSL5-IM5 (90 mg, 0.31 mmol, 1.0 eq) dissolved in 1.0 mL of THF, washed twice with 0.5 mL of THF, and the reaction mixture was reacted overnight at room temperature. TLC (CH₂Cl₂:MeOH=50:1) was used to monitor the reaction until the starting material disappeared completely. The reaction mixture was concentrated, and the residue was purified by PTLC (CH₂Cl₂:MeOH=50:1) to obtain the product (85 mg, yield of 95%).

### Step 7

Compound SSL5-IM6 (85 mg, 0.28 mmol, 1.0 eq) was taken and dissolved in 2.0 mL of THF, and 1.0 mL of MeOH was added thereto. LiOH (35 mg, 0.84 mmol, 3.0 eq) was added to another EP tube, dissolved in 1.0 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=10:1) to obtain the product (44 mg, yield of 57%). 1H NMR (400 MHz, Methanol-d4) δ 7.36 (s, 1H), 7.09 (s, 1H), 7.02 (s, 2H), 6.64 (dd, J=8.0, 1.7 Hz, 1H), 5.05 (s, 2H), 2.46-2.41 (m, 1H), 2.36 (s, 3H), 1.67-1.63 (m, 1H), 1.52-1.47 (m, 1H), 1.37-1.32 (m, 1H). Mass: [M+H]⁺ 275.1.

### Step 8

SSL5-IM7 was obtained and purified by a chiral preparative column to obtain compound 5 and Compound 25.

### Example 6 Synthesis of Compound 6 and Compound 26

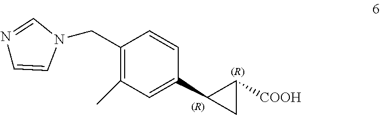

### (1R,2R)-2-(4-((1H-Imidazol-1-yl)methyl)-3-methylphenyl)cyclopropane-1-carboxylic acid

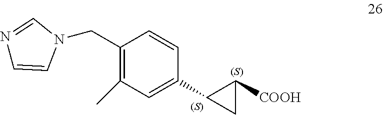

### (1S,2S)-2-(4-((1H-Imidazol-1-yl)methyl)-3-methylphenyl)cyclopropane-1-carboxylic acid

### Synthetic Route

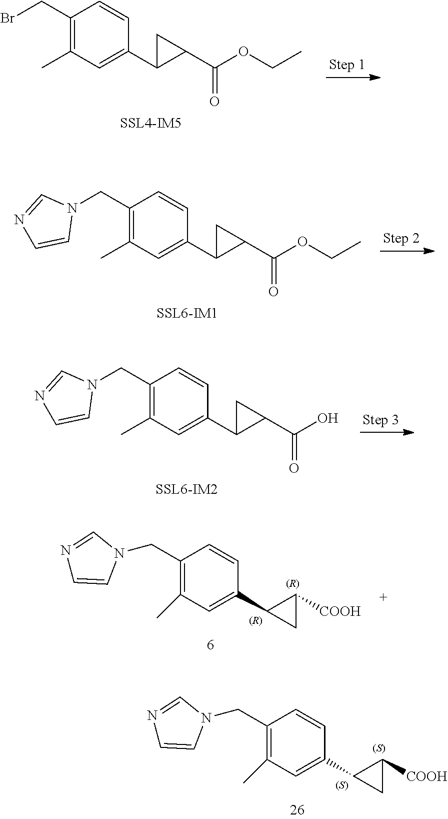

### Step 1

The crude product of SSL4-IM5 was taken and dissolved in 3.0 mL of CH₃CN, and imidazole (27 mg, 0.40 mmol, 2.0 eq) and K₂CO₃ (55 mg, 0.40 mmol, 2.0 eq) were added thereto, and then the reaction mixture was reacted at 40° C. for 4.0 hours. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (40 mg, yield of 70%).

### Step 2

Compound SSL6-IM1 (40 mg, 0.14 mmol, 1.0 eq) was taken and dissolved in 1.0 mL of THF, and 0.5 mL of MeOH was added thereto. LiOH (12 mg, 0.28 mmol, 2.0 eq) was added to another EP tube, dissolved in 0.5 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=5:1) to obtain the product (25 mg, yield of 70%). 1H NMR (400 MHz, Methanol-d4) δ 7.77 (s, 1H), 7.07-6.95 (m, 5H), 5.23 (s, 2H), 2.45-2.37 (m, 1H), 2.24 (s, 3H), 1.84-1.78 (m, 1H), 1.53-1.47 (m, 1H), 1.33-1.31 (m, 1H). Mass: [M+H]⁺ 257.1.

### Step 3

SSL6-IM2 was obtained and purified by a chiral preparative column to obtain compound 6 and Compound 26.

### Example 7 Synthesis of Compound 7 and Compound 27

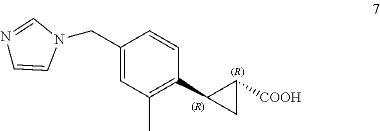

### (1R,2R)-2-(4-((1H-Imidazol-1-yl)methyl)-2-methylphenyl)cyclopropane-1-carboxylic acid

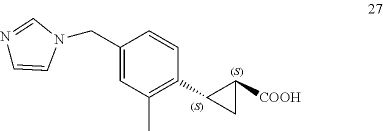

### (1S,2S)-2-(4-((1H-Imidazol-1-yl)methyl)-2-methylphenyl)cyclopropane-1-carboxylic acid

### Synthetic Route

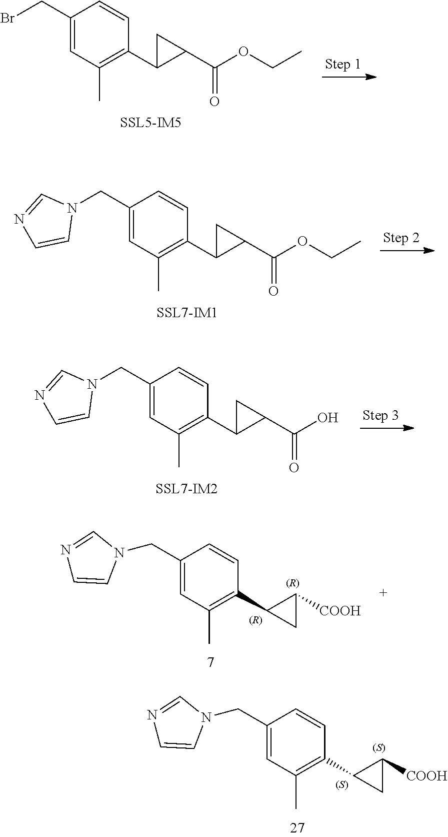

### Step 1

SSL5-IM5 (92 mg, 0.31 mmol, 1.0 eq) was taken and dissolved in 4.0 mL of CH₃CN, and imidazole (62 mg, 0.93 mmol, 3.0 eq) and K₂CO₃ (126 mg, 0.93 mmol, 3.0 eq) were added thereto, and then the reaction mixture was reacted at 45° C. for 6.0 hours. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (61 mg, yield of 69%).

### Step 2

Compound SSL7-IM1 (61 mg, 0.21 mmol, 1.0 eq) was taken and dissolved in 1.0 mL of THF, and 0.5 mL of MeOH was added thereto. LiOH (18 mg, 0.14 mmol, 2.0 eq) was added to another EP tube, dissolved in 0.5 mL of H₂O, which was added to the reaction mixture and reacted at room temperature for 6.0 hours. TLC (DCM:MeOH=10:1) was used to monitor the reaction until the starting material disappeared completely, and the reaction mixture was concentrated, and then the residue was purified by PTLC (CH₂Cl₂:MeOH=10:1) to obtain the product (32 mg, yield of 60%). 1H NMR (400 MHz, Methanol-d4) δ 7.85 (s, 1H), 7.13 (t, J=1.4 Hz, 1H), 7.08 (s, 1H), 7.03-7.02 (m, 3H), 5.16 (s, 2H), 2.45-2.40 (m, 1H), 2.37 (s, 3H), 1.65-1.60 (m, 1H), 1.49-1.45 (m, 1H), 1.32-1.30 (m, 1H). Mass: [M+H]⁺ 257.1.

### Step 3

SSL7-IM2 was obtained and purified by a chiral preparative column to obtain compound 7 and Compound 27.

### Example 8 Synthesis of Compound 8 and Compound 28

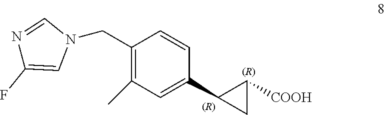

### (1R,2R)-2-(4-((4-Fluoro-1H-imidazol-1-yl)methyl)-3-methylphenyl)cyclopropane-1-carboxylic acid

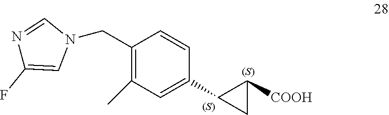

### (1S,2S)-2-(4-((4-Fluoro-1H-imidazol-1-yl)methyl)-3-methylphenyl)cyclopropane-1-carboxylic acid

### Synthetic Route

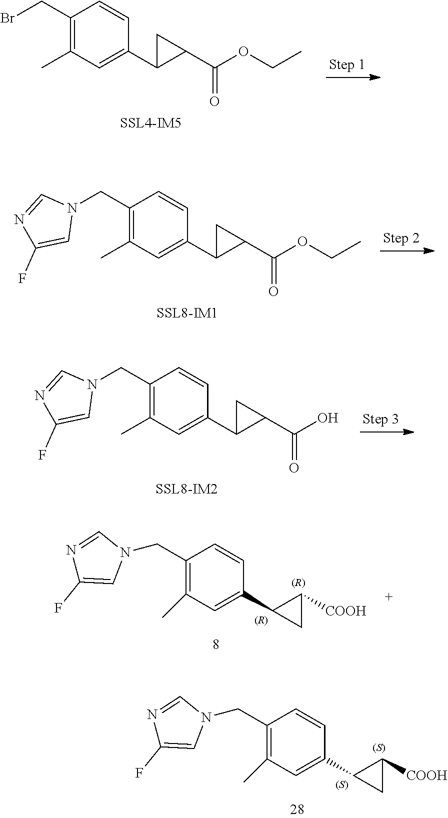

### Step 1

The starting material was dissolved in 1.5 mL of THF, added to a reaction flask, and 5-fluoroimidazole (45 mg, 0.50 mmol, 2.5 eq) and NaH (20 mg, 0.50 mmol, 2.5 eq) were added thereto, reacted for 0.5 hours at room temperature, added with a solution of SSL4-IM5 (60 mg, 0.20 mmol, 1.0 eq) dissolved in 1.0 mL of THF, washed with 0.5 mL of THF, and the reaction mixture was reacted overnight at room temperature. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (50 mg, yield of 86%).

### Step 2

Compound SSL8-IM1 (50 mg, 0.173 mmol, 1.0 eq) was taken and dissolved in 1.0 mL of THF, and 0.5 mL of MeOH was added thereto. LiOH (15 mg, 0.35 mmol, 2.0 eq) was added to another EP tube, dissolved in 0.5 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=10:1) to obtain the product (32 mg, yield of 67%). 1H NMR (400 MHz, Methanol-d4) δ 7.26 (s, 1H), 7.10-6.93 (m, 3H), 6.57 (dd, J=7.9, 1.6 Hz, 1H), 5.12 (s, 2H), 2.43 (ddd, J=9.8, 6.3, 4.1 Hz, 1H), 2.24 (s, 3H), 1.82 (dt, J=8.9, 4.7 Hz, 1H), 1.51 (dt, J=9.5, 4.9 Hz, 1H), 1.36-1.31 (m, 1H). Mass: [M+H]⁺ 275.1.

### Step 3

SSL8-IM2 was obtained and purified by a chiral preparative column to obtain compound 8 and Compound 28.

### Example 9 Synthesis of Compound 9 and Compound 29

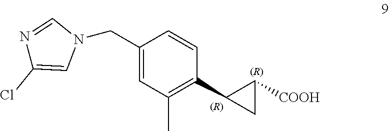

### (1R,2R)-2-(4-((4-Chloro-1H-imidazol-1-yl)methyl)-2-methylphenyl)cyclopropane-1-carboxylic acid

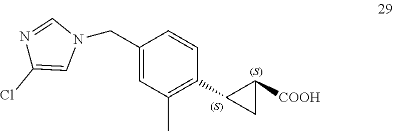

### (1S,2S)-2-(4-((4-Chloro-1H-imidazol-1-yl)methyl)-2-methylphenyl)cyclopropane-1-carboxylic acid

### Synthetic Route

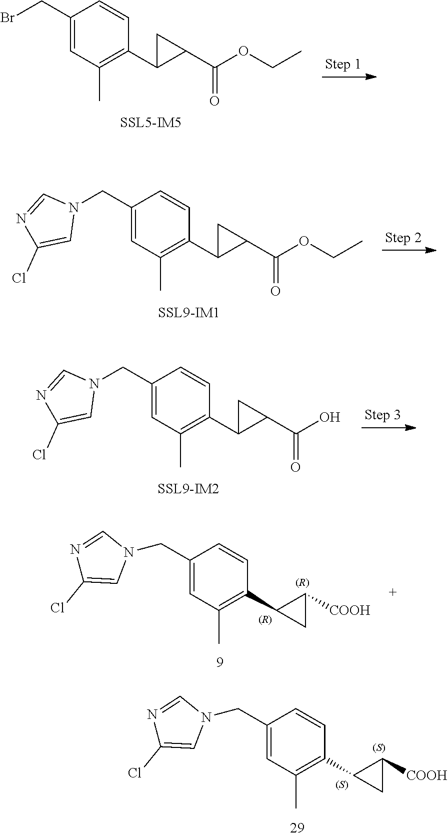

### Step 1

The starting material was dissolved in 2.0 mL of THE and added to a reaction flask, and then 5-chloroimidazole (93 mg, 0.93 mmol, 3.0 eq) and NaH (36 mg, 0.93 mmol, 3.0 eq) were added thereto, reacted for 0.5 hours at room temperature, added with a solution of SSL5-IM5 (90 mg, 0.31 mmol, 1.0 eq) in 1.0 mL of THF, washed twice with 0.5 mL of THF, and the reaction mixture was reacted overnight at room temperature. TLC (CH₂Cl₂:MeOH=50:1) was used to monitor the reaction until the starting material disappeared completely. The reaction mixture was concentrated, and the residue was purified by PTLC (CH₂Cl₂:MeOH=50:1) to obtain the product (77 mg, yield of 82%).

### Step 2

Compound SSL9-IM1 (77 mg, 0.24 mmol, 1.0 eq) was taken and dissolved in 2.0 mL of THF, and 1.0 mL of MeOH was added thereto. LiOH (30 mg, 0.72 mmol, 3.0 eq) was added to another EP tube, dissolved in 1.0 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=10:1) to obtain the product (34 mg, yield of 49%). 1H NMR (400 MHz, Methanol-d4) δ 7.63 (s, 1H), 7.10 (s, 1H), 7.04-7.02 (m, 3H), 5.08 (s, 2H), 2.48-2.40 (m, 1H), 2.36 (s, 3H), 1.68-1.63 (m, 1H), 1.52-1.47 (m, 1H), 1.38-1.32 (m, 1H). Mass: [M+H]⁺ 291.1.

### Step 3

SSL9-IM2 was obtained and purified by a chiral preparative column to obtain compound 9 and Compound 29.

### Example 10 Synthesis of Compound 10 and Compound 30

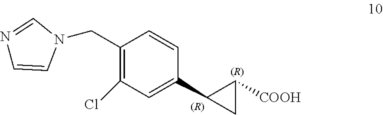

### (1R,2R)-2-(4-((1H-Imidazol-1-yl)methyl)-3-chlorophenyl)cyclopropane-1-carboxylic acid

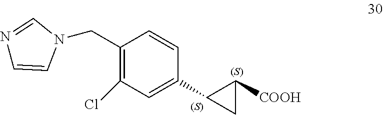

### (1S,2S)-2-(4-((1H-Imidazol-1-yl)methyl)-3-chlorophenyl)cyclopropane-1-carboxylic acid

### Synthetic Route

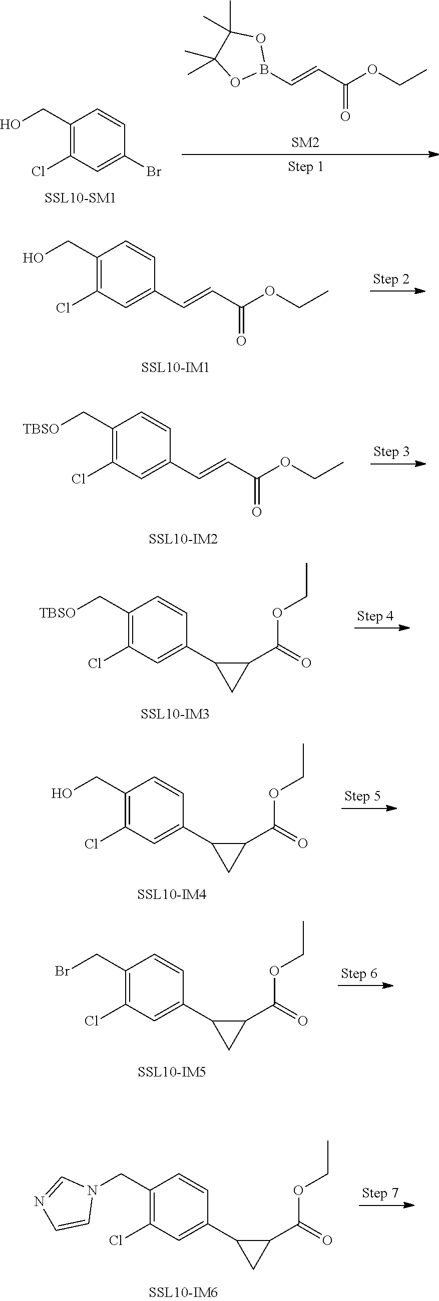

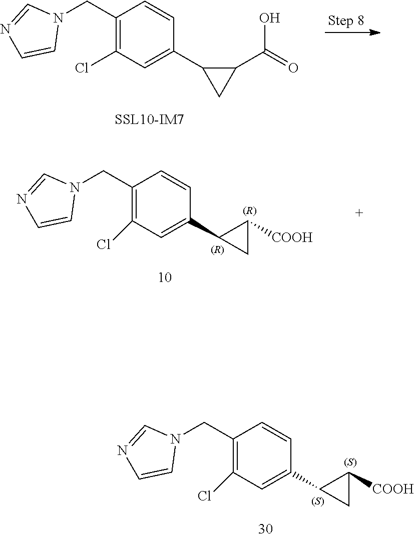

### Step 1

SSL10-SM1 (2.21 g, 10 mmol, 1.0 eq), SM2 (3.39 g, 15 mmol, 1.5 eq), Pd(dppf)Cl₂ (731 mg, 1 mmol, 0.1 eq), and potassium carbonate (4.1 g, 30 mmol, 3.0 eq) were added to a 100 mL single-neck flask under N₂ atmosphere, and dioxane and water were added thereto, and then the reaction mixture was heated to 100° C. and stirred for 2 hours. The complete reaction of the raw material was monitored by TLC (PE/EA 3:1). After the reaction was complete, the reaction mixture was subjected to rotary evaporation until dryness, and the residue was subjected to column chromatography (DCM/MeOH 50:1) to obtain a light yellow liquid (1.6 g, yield of 72%).

### Step 2

SSL10-IM1 (529 mg, 2.2 mmol, 1.0 eq) was dissolved in DCM at room temperature, and imidazole (300 mg, 4.4 mmol, 2.0 eq) was added thereto, and then TBSCl (406 mg, 2.7 mmol, 1.2 eq) was added dropwise thereto; the reaction mixture was stirred for 3 hours after the addition was complete. The complete reaction of the raw material was monitored by TLC (PE/EA 6:1). The solvent was removed by rotary evaporation until dryness, and the residue was subjected to column chromatography (PE/EA 10:1) to obtain the product (388 mg, yield of 52%).

### Step 3

Trimethylsulfoxonium iodide (252 mg, 1.14 mmol, 1.0 eq), DMSO (6 mL), and NaH (54 mg, 1.36 mmol, 1.2 eq) were added to a 50 mL single-neck flask under N₂ atmosphere. The reaction mixture was stirred for 1.5 hours at room temperature. Then all the prepared Ylide was added dropwise to a solution of SSL10-IM2 (388 mg, 1.14 mmol, 1.0 eq) in DMSO and stirred at room temperature, and the complete reaction of the raw material was monitored by TLC (PE/EA 10:1). After the reaction was complete, the reaction was quenched with a small amount of water, and the reaction mixture was extracted with ethyl acetate, washed three times with saturated brine. The organic phase was subjected to rotary evaporation until dryness, and the residue was used directly in the next step.

### Step 4

SSL10-IM3 (50 mg, 0.14 mmol, 1.0 eq) was dissolved in THF, and TBAF (0.14 mL, 0.14 mmol, 1.0 eq) was added thereto, and then the reaction mixture was stirred at room temperature for 2 hours. After the reaction was complete, the reaction mixture was subjected to rotary evaporation until dryness, and the residue was subjected to column chromatography (PE/EA 5:1) to obtain a light yellow liquid.

### Step 5

SSL10-IM4 (50 mg, 0.23 mmol, 1.0 eq) was dissolved in dichloromethane, and tetrabromomethane (90 mg, 0.27 mmol, 1.2 eq) was added thereto, maintained at 0° C., and triphenylphosphine (71 mg, 0.27 mmol, 1.2 eq) was added thereto in batches, still maintained at 0° C., and then the reaction mixture was stirred for 2 hours at 0° C. After the reaction was complete, the reaction mixture was subjected to rotary evaporation until dryness, and the residue was subjected to column chromatography (PE/EA 10:1) to obtain a light yellow solid (25 mg, yield of 38%).

### Step 6

SSL10-IM5 (60 mg, 0.20 mmol, 1.0 eq), potassium carbonate (55 mg, 0.40 mmol, 2.0 eq), and imidazole (27 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 7

SSL10-IM6 (45 mg, 0.16 mmol, 1.0 eq) was dissolved in tetrahydrofuran and methanol, then the mixture was added dropwise with lithium hydroxide aqueous solution (13 mg, 0.31 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.74 (s, 1H), 7.16 (t, J=7.9 Hz, 1H), 7.10 (s, 1H), 6.99-6.86 (m, 3H), 5.22 (s, 2H), 2.35 (ddd, J=9.6, 6.0, 4.1 Hz, 1H), 1.76 (dt, J=9.1, 4.9 Hz, 1H), 1.47 (dt, J=9.4, 4.9 Hz, 1H), 1.16 (ddd, J=8.5, 6.1, 4.3 Hz, 1H). Mass: [M+H]⁺ 277.0.

### Step 8

SSL10-IM7 was obtained and purified by a chiral preparative column to obtain compound 10 and Compound 30.

### Example 11 Synthesis of Compound 11 and Compound 31

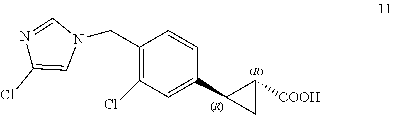

### (1R,2R)-2-(3-Chloro-4-((4-chloro-1H-imidazol-1-yl)methyl)phenyl)cyclopropane-1-carboxylic acid

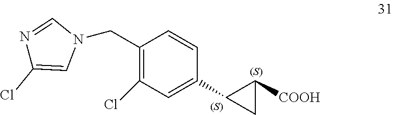

### (1S,2S)-2-(3-Chloro-4-((4-chloro-1H-imidazol-1-yl)methyl)phenyl)cyclopropane-1-carboxylic acid

### Synthetic Route

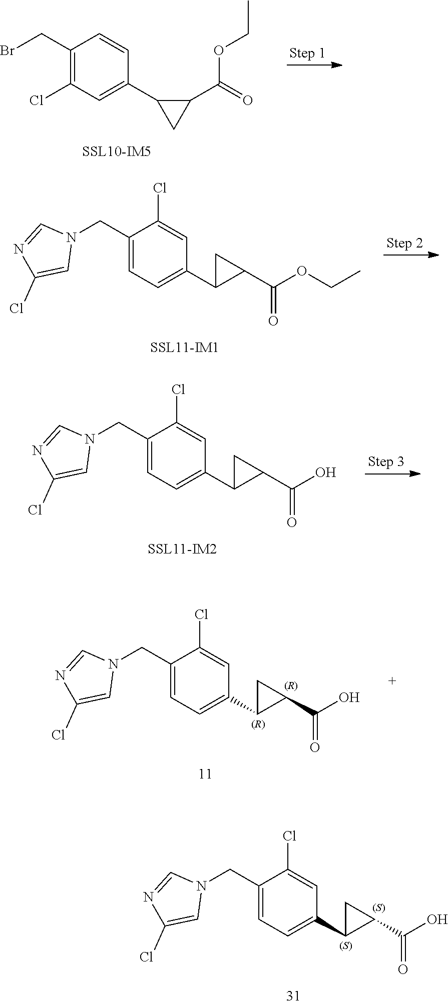

### Step 1

SSL10-IM5 (0.3 g, 0.945 mmol, 1.0 eq), potassium carbonate (0.52 g, 3.78 mmol, 4.0 eq), and 5-chloroimidazole (0.29 g, 2.83 mmol, 3.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (120 mg, yield of 38%).

### Step 2

SSL11-IM1 (0.12 g, 0.354 mmol, 1.0 eq) was dissolved in tetrahydrofuran and methanol, then the mixture was added dropwise with lithium hydroxide aqueous solution (0.03 g, 0.71 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (104 mg, yield of 95%). ¹H NMR (400 MHz, MeOD) δ 7.64 (d, J=1.1 Hz, 1H), 7.21 (d, J=1.3 Hz, 1H), 7.16 (d, J=8.0 Hz, 1H), 7.11-6.95 (m, 2H), 5.25 (s, 2H), 2.41-2.25 (m, 1H), 1.84-1.66 (m, 1H), 1.56-1.41 (m, 1H), 1.22-1.05 (m, 1H). Mass: [M+H]⁺ 311.1.

### Step 3

SSL11-IM2 was obtained and purified by a chiral preparative column to obtain compound 11 and Compound 31.

Preparation method of a single crystal of compound 11: 3 mg of the target compound 11 was taken and placed in a 2.0 mL liquid phase vial, and 0.5 mL of CH₂Cl₂ was added thereto, then a solid suspension was seen. 3 to 4 drops of MeOH were added thereto, and the solid was dissolved. The vial was sealed with plastic wrap, and several small holes were pricked with a needle. The vial was placed in a 20 mL brown sample bottle containing 4.0 mL of n-hexane, and the brown sample bottle was sealed and placed in a refrigerator (2 to 8° C.) for 48 hours, and the crystal was observed to precipitate.

The structure data of a single crystal of compound 11 are as follows:

| Parameters | Single crystal of compound 11 |
|---|---|
| Experimental formula | C14H12Cl2N2O2 |
| Molecular weight | 311.16 |
| Temperature | 113.15 K |
| Wavelength | 0.71073 A |
| Crystal system | Hexagonal |
| Space group | P 61 |
| Crystal cell size | a = 10.5982(3) Å, α = 90°. |
| | b = 10.5982(3) Å, β = 90°. |
| | c = 21.6750(8) Å, γ = 120°. |
| Crystal cell volume | 2108.40(14) Å³ |
| Z | 6 |
| Density (calculated) | 1.470 Mg/m³ |
| Absorption coefficient | 0.463 mm⁻¹ |
| F(000) | 960.0 |
| Crystal size | 0.21 × 0.19 × 0.15 mm³ |
| θ range for data collection | 4.438 to 67.122° |
| Range of indicators | −16 ≤ h ≤ 16, −16 ≤ k ≤ 16, −29 ≤ l < 32 |
| Diffraction point collection | 25979 |
| Independent diffraction points | 5172 [Rint = 0.0599, Rsigma = 0.0513] |
| Refinement method | Goodness-of-fit on F² |

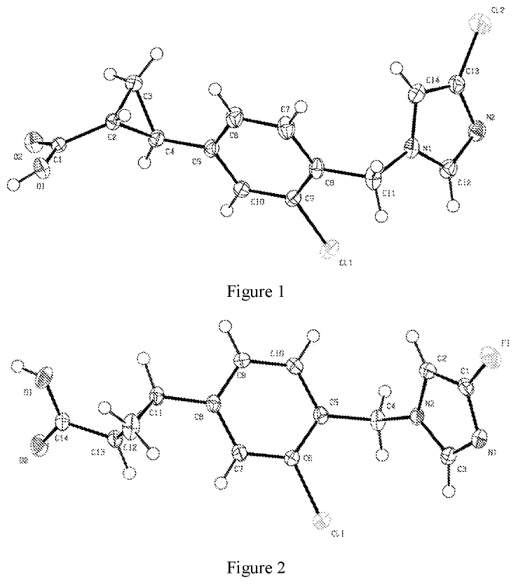

The single crystal structure of compound 11 is shown in FIG. 1.

### Example 12 Synthesis of Compound 12 and Compound 32

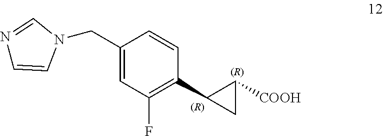

### (1R,2R)-2-(4-((1H-Imidazol-1-yl)methyl)-2-fluorophenyl)cyclopropane-1-carboxylic acid

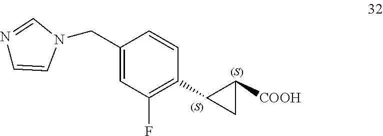

### (1S,2S)-2-(4-((1H-Imidazol-1-yl)methyl)-2-fluorophenyl)cyclopropane-1-carboxylic acid

### Synthetic Route

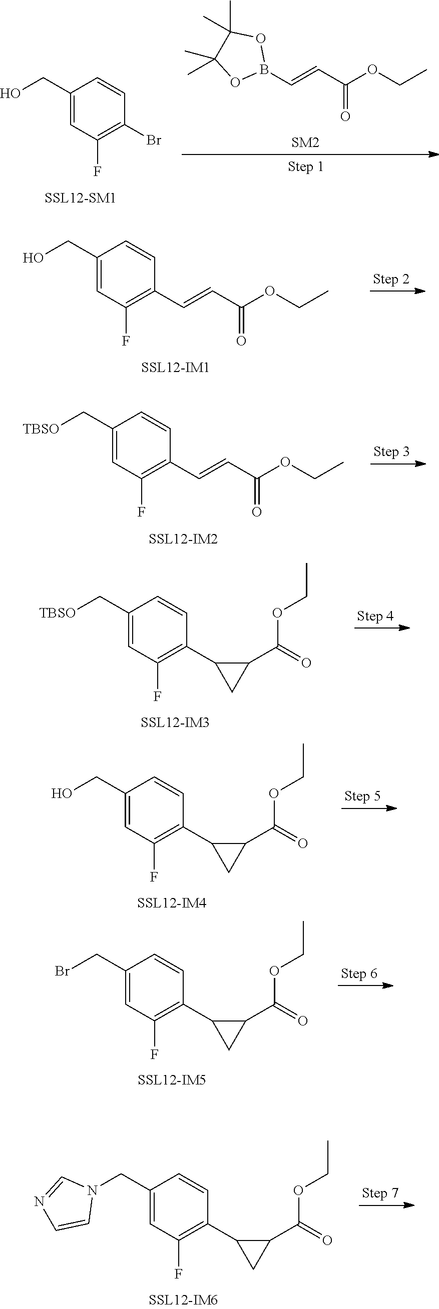

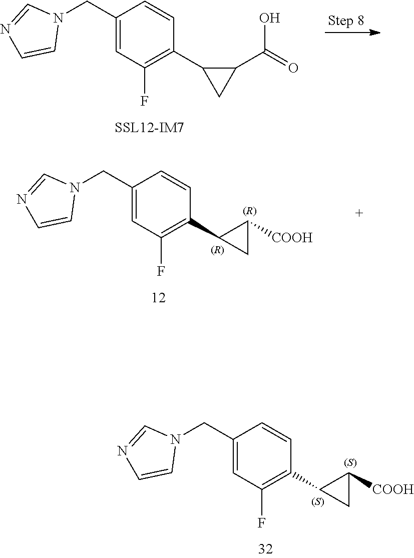

### Step 1

SSL12-SM1 (2 g, 10 mmol, 1.0 eq), SM2 (3.3 g, 15 mmol, 1.5 eq), Pd(dppf)Cl₂ (731 mg, 1 mmol, 0.1 eq), and potassium carbonate (4.1 g, 30 mmol, 3.0 eq) were added to a 100 mL single-neck flask under N₂ atmosphere, and dioxane and water were added thereto, and then the reaction mixture was heated to 100° C. and stirred for 2 hours. The complete reaction of the raw material was monitored by TLC (PE/EA 3:1). After the reaction was complete, the reaction mixture was subjected to rotary evaporation until dryness, and the residue was subjected to column chromatography (DCM/MeOH 50:1) to obtain a light yellow liquid (1.6 g, yield of 72%).

### Step 2

SSL12-IM1 was dissolved in DCM at room temperature, and imidazole (300 mg, 4.4 mmol, 2.0 eq) was added thereto, and then TBSCl (403 mg, 2.7 mmol, 1.2 eq) was added dropwise thereto; the reaction mixture was stirred for 3 hours after the addition was complete. The complete reaction of the raw material was monitored by TLC (PE/EA 6:1). The solvent was removed by rotary evaporation until dryness, and the residue was subjected to column chromatography (PE/EA 10:1) to obtain the product (388 mg, yield of 52%).

### Step 3

Trimethylsulfoxonium iodide (660 mg, 3 mmol, 1.0 eq), DMSO (6 mL), and NaH (144 mg, 3.6 mmol, 1.1 eq) were added to a 50 mL single-neck flask under N₂ atmosphere. The reaction mixture was stirred for 1.5 hours at room temperature. Then all the prepared Ylide (0.24 mL) was added dropwise to a solution of SSL12-IM2 (50 mg, 0.12 mmol, 1.0 eq) in DMSO and stirred at room temperature, and the complete reaction of the raw material was monitored by TLC (PE/EA 10:1). After the reaction was complete, the reaction was quenched with a small amount of water, and the reaction mixture was extracted with ethyl acetate, washed three times with saturated brine. The organic phase was subjected to rotary evaporation until dryness, and the residue was used directly in the next step.

### Step 4

SSL12-IM3 (50 mg, 0.14 mmol, 1.0 eq) was dissolved in THF, then TBAF (0.14 mL, 0.14 mmol, 1.0 eq) was added thereto, and the reaction mixture was stirred at room temperature for 2 hours. After the reaction was complete, the reaction mixture was subjected to rotary evaporation until dryness, and the residue was subjected to column chromatography (PE/EA 10:1) to obtain a light yellow liquid.

### Step 5

SSL12-IM4 (50 mg, 0.23 mmol, 1.0 eq) was dissolved in dichloromethane, and tetrabromomethane (90 mg, 0.27 mmol, 1.2 eq) was added thereto, maintained at 0° C., and triphenylphosphine (71 mg, 0.27 mmol, 1.2 eq) was added thereto in batches, still maintained at 0° C., and then the reaction mixture was stirred for 2 hours at 0° C. After the reaction was complete, the reaction mixture was subjected to rotary evaporation until dryness, and the residue was subjected to column chromatography (PE/EA 10:1) to obtain a light yellow solid (25 mg, yield of 38%).

### Step 6

SSL12-IM5 (60 mg, 0.20 mmol, 1.0 eq), potassium carbonate (55 mg, 0.40 mmol, 2.0 eq), and 4-chloroimidazole (41 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 7

SSL12-IM6 (45 mg, 0.16 mmol, 1.0 eq) was dissolved in tetrahydrofuran and methanol, then the mixture was added dropwise with lithium hydroxide aqueous solution (13 mg, 0.31 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.86 (s, 1H), 7.16 (s, 1H), 7.09-6.92 (m, 4H), 5.22 (s, 2H), 2.53 (dd, J=6.4, 2.9 Hz, 1H), 1.54-1.44 (m, 1H), 1.31 (ddd, J=8.0, 6.2, 4.1 Hz, 2H). Mass: [M+H]⁺ 261.1.

### Step 8

SSL12-IM7 was obtained and purified by a chiral preparative column to obtain compound 12 and Compound 32.

### Example 13 Synthesis of Compound 13 and Compound 33

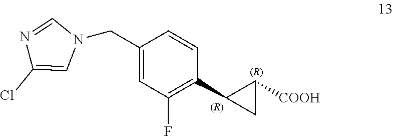

### (1R,2R)-2-(4-((4-Chloro-1H-imidazol-1-yl)methyl)-2-fluorophenyl)cyclopropane-1-carboxylic acid

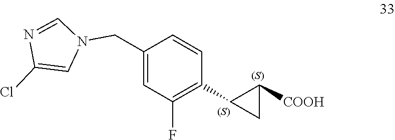

### (1S,2S)-2-(4-((4-Chloro-1H-imidazol-1-yl)methyl)-2-fluorophenyl)cyclopropane-1-carboxylic acid

### Synthetic Route

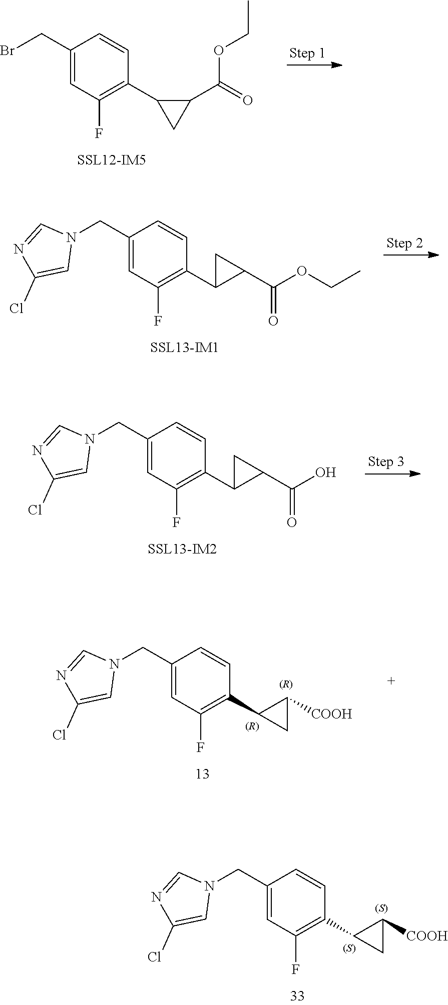

### Step 1

SSL13-SM (60 mg, 0.20 mmol, 1.0 eq), potassium carbonate (55 mg, 0.40 mmol, 2.0 eq), and 4-chloroimidazole (41 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 2

SSL13-IM1 (45 mg, 0.14 mmol, 1.0 eq) was dissolved in tetrahydrofuran and methanol, then the mixture was added dropwise with lithium hydroxide aqueous solution (12 mg, 0.28 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.68 (d, J=4.9 Hz, 1H), 7.10 (d, J=5.0 Hz, 1H), 7.07-6.99 (m, 3H), 5.15 (s, 2H), 2.54 (p, J=4.9 Hz, 1H), 1.87-1.76 (m, 1H), 1.51-1.42 (m, 1H), 1.31-1.27 (m, 1H). Mass: [M+H]⁺ 295.1.

### Step 3

SSL13-IM2 was obtained and purified by a chiral preparative column to obtain compound 13 and Compound 33.

### Example 14 Synthesis of Compound 14 and Compound 34

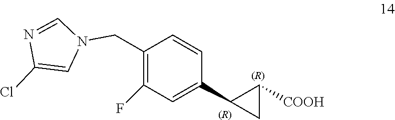

### (1R,2R)-2-(4-((4-Chloro-1H-imidazol-1-yl)methyl)-3-fluorophenyl)cyclopropane-1-carboxylic acid

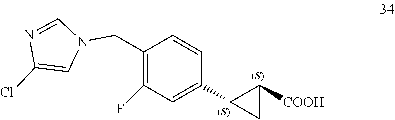

### (1S,2S)-2-(4-((4-Chloro-1H-imidazol-1-yl)methyl)-3-fluorophenyl)cyclopropane-1-carboxylic acid

### Synthetic Route

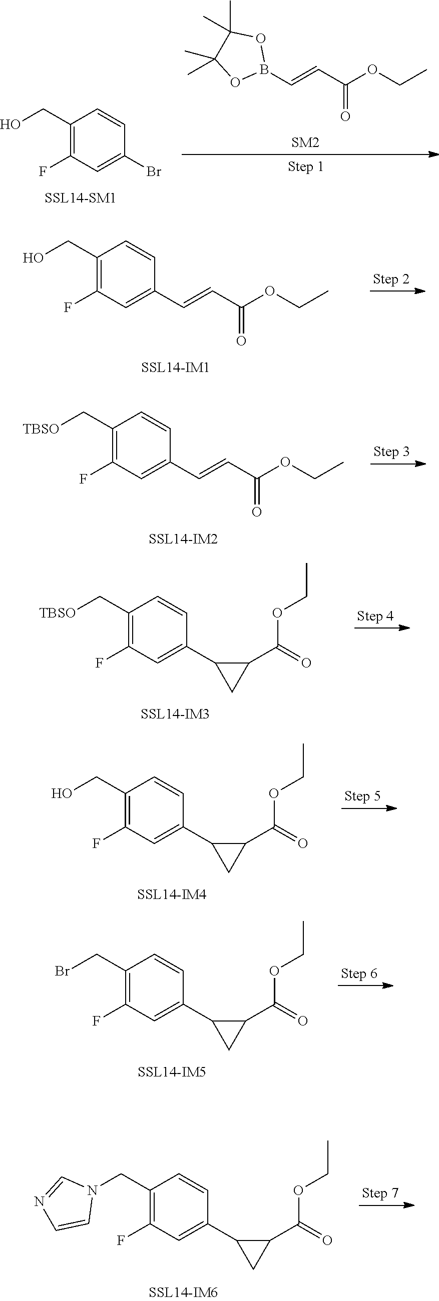

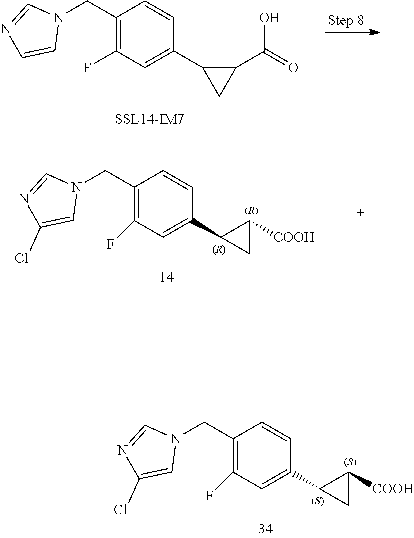

### Step 1

SSL14-SM1 (2 g, 10 mmol, 1.0 eq), SM2 (3.3 g, 15 mmol, 1.5 eq), Pd(dppf)Cl₂ (731 mg, 1 mmol, 0.1 eq), and potassium carbonate (4.1 g, 30 mmol, 3.0 eq) were added to a 100 mL single-neck flask under N₂ atmosphere, and dioxane and water were added thereto, and then the reaction mixture was heated to 100° C. and stirred for 2 hours. The complete reaction of the raw material was monitored by TLC (PE/EA 3:1). After the reaction was complete, the reaction mixture was subjected to rotary evaporation until dryness, and the residue was subjected to column chromatography (DCM/MeOH 50:1) to obtain a light yellow liquid (1.6 g, yield of 72%).

### Step 2

SSL14-IM1 was dissolved in DCM at room temperature, and imidazole (300 mg, 4.4 mmol, 2.0 eq) was added thereto, and then TBSCl (403 mg, 2.7 mmol, 1.2 eq) was added dropwise thereto; the reaction mixture was stirred for 3 hours after the addition was complete. The complete reaction of the raw material was monitored by TLC (PE/EA 6:1). The solvent was removed by rotary evaporation until dryness, and the residue was subjected to column chromatography (PE/EA 10:1) to obtain the product (388 mg, yield of 52%).

### Step 3

Trimethylsulfoxonium iodide (660 mg, 3 mmol, 1.0 eq), DMSO (6 mL), and NaH (144 mg, 3.6 mmol, 1.1 eq) were added to a 50 mL single-neck flask under N₂ atmosphere. The reaction mixture was stirred for 1.5 hours at room temperature. Then all the prepared Ylide (0.24 mL) was added dropwise to a solution of SSL14-IM2 (50 mg, 0.12 mmol, 1.0 eq) in DMSO and stirred at room temperature, and the complete reaction of the raw material was monitored by TLC (PE/EA 10:1). After the reaction was complete, the reaction was quenched with a small amount of water, and the reaction mixture was extracted with ethyl acetate, washed three times with saturated brine. The organic phase was subjected to rotary evaporation until dryness, and the residue was used directly in the next step.

### Step 4

SSL14-IM3 (50 mg, 0.14 mmol, 1.0 eq) was dissolved in THF, then TBAF (0.14 mL, 0.14 mmol, 1.0 eq) was added thereto, and the reaction mixture was stirred at room temperature for 2 hours. After the reaction was complete, the reaction mixture was subjected to rotary evaporation until dryness, and the residue was subjected to column chromatography (PE/EA 10:1) to obtain a light yellow liquid.

### Step 5

SSL14-IM4 (50 mg, 0.23 mmol, 1.0 eq) was dissolved in dichloromethane, and tetrabromomethane (90 mg, 0.27 mmol, 1.2 eq) was added thereto, maintained at 0° C., and triphenylphosphine (71 mg, 0.27 mmol, 1.2 eq) was added thereto in batches, still maintained at 0° C., and then the reaction mixture was stirred for 2 hours at 0° C. After the reaction was complete, the reaction mixture was subjected to rotary evaporation until dryness, and the residue was subjected to column chromatography (PE/EA 10:1) to obtain a light yellow solid (25 mg, yield of 38%).

### Step 6

SSL14-IM5 (60 mg, 0.20 mmol, 1.0 eq), potassium carbonate (55 mg, 0.40 mmol, 2.0 eq), and 4-chloroimidazole (41 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 7

SSL14-IM6 (45 mg, 0.14 mmol, 1.0 eq) was dissolved in tetrahydrofuran and methanol, then the mixture was added dropwise with lithium hydroxide aqueous solution (12 mg, 0.28 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.64 (d, J=1.5 Hz, 1H), 7.22 (t, J=7.9 Hz, 1H), 7.06 (d, J=1.6 Hz, 1H), 7.01-6.93 (m, 2H), 5.19 (s, 2H), 2.46 (ddd, J=10.0, 6.4, 3.9 Hz, 1H), 1.86 (s, 1H), 1.54 (dt, J=9.5, 4.9 Hz, 1H), 1.34 (ddd, J=8.9, 5.2, 3.3 Hz, 1H). Mass: [M+H]⁺ 295.0.

### Step 8

SSL14-IM7 was obtained and purified by a chiral preparative column to obtain compound 14 and Compound 34.

### Example 15 Synthesis of Compound 15 and Compound 35

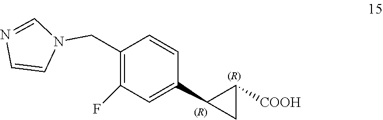

### (1R,2R)-2-(4-((1H-Imidazol-1-yl)methyl)-3-fluorophenyl)cyclopropane-1-carboxylic acid

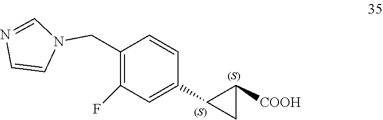

### (1S,2S)-2-(4-((1H-Imidazol-1-yl)methyl)-3-fluorophenyl)cyclopropane-1-carboxylic acid

### Synthetic Route

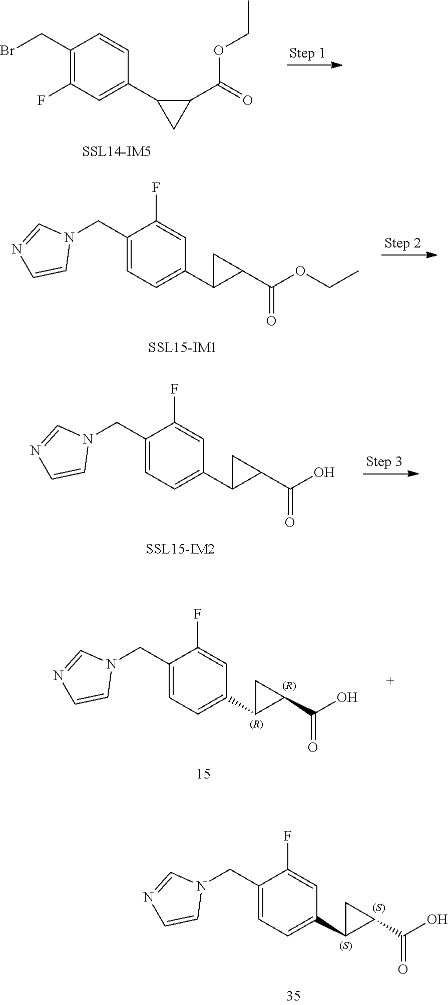

### Step 1

SSL14-IM5 (60 mg, 0.20 mmol, 1.0 eq), potassium carbonate (55 mg, 0.40 mmol, 2.0 eq), and imidazole (27 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 2

SSL15-IM1 (45 mg, 0.16 mmol, 1.0 eq) was dissolved in tetrahydrofuran and methanol, then the mixture was added dropwise with lithium hydroxide aqueous solution (13 mg, 0.31 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.74 (s, 1H), 7.16 (t, J=7.9 Hz, 1H), 7.10 (s, 1H), 6.99-6.86 (m, 3H), 5.22 (s, 2H), 2.35 (ddd, J=9.6, 6.0, 4.1 Hz, 1H), 1.76 (dt, J=9.1, 4.9 Hz, 1H), 1.47 (dt, J=9.4, 4.9 Hz, 1H), 1.16 (ddd, J=8.5, 6.1, 4.3 Hz, 1H). Mass: [M+H]⁺ 261.1.

### Step 3

SSL15-IM2 was obtained and purified by a chiral preparative column to obtain compound 15 and Compound 35.

### Example 16 Synthesis of Compound 16 and Compound 36

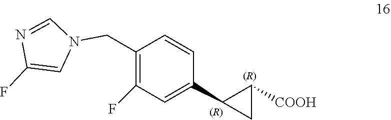

### (1R,2R)-2-(3-Fluoro-4-((4-fluoro-1H-imidazol-1-yl)methyl)phenyl)cyclopropane-1-carboxylic acid

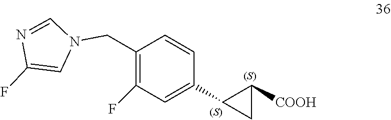

### (1S,2S)-2-(3-Fluoro-4-((4-fluoro-1H-imidazol-1-yl)methyl)phenyl)cyclopropane-1-carboxylic acid

### Synthetic Route

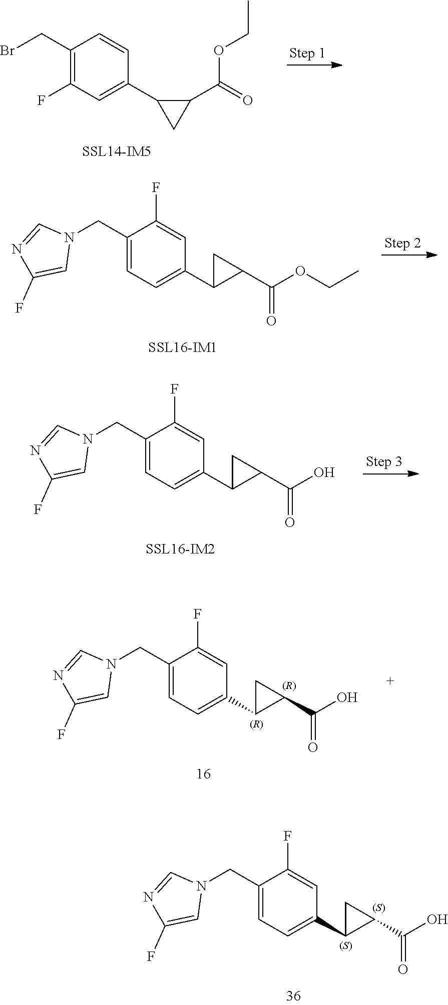

### Step 1

SSL14-IM5 (60 mg, 0.20 mmol, 1.0 eq), potassium carbonate (55 mg, 0.40 mmol, 2.0 eq), and 4-fluorimidazole (41 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 2

SSL16-IM1 (45 mg, 0.14 mmol, 1.0 eq) was dissolved in tetrahydrofuran and methanol, then the mixture was added dropwise with lithium hydroxide aqueous solution (12 mg, 0.28 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.64 (d, J=1.5 Hz, 1H), 7.22 (t, J=7.9 Hz, 1H), 7.06 (d, J=1.6 Hz, 1H), 7.01-6.93 (m, 2H), 5.19 (s, 2H), 2.46 (ddd, J=10.0, 6.4, 3.9 Hz, 1H), 1.86 (s, 1H), 1.54 (dt, J=9.5, 4.9 Hz, 1H), 1.34 (ddd, J=8.9, 5.2, 3.3 Hz, 1H). Mass: [M+H]⁺ 279.1.

### Step 3

SSL16-IM2 was obtained and purified by a chiral preparative column to obtain compound 16 and Compound 36.

### Example 17 Synthesis of Compound 17 and Compound 37

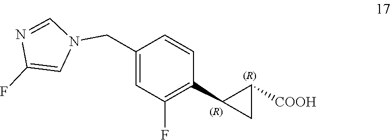

### (1R,2R)-2-(2-Fluoro-4-((4-fluoro-1H-imidazol-1-yl)methyl)phenyl)cyclopropane-1-carboxylic acid

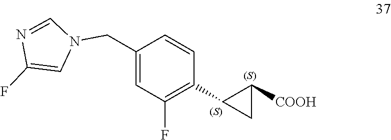

### (1S,2S)-2-(2-Fluoro-4-((4-fluoro-1H-imidazol-1-yl)methyl)phenyl)cyclopropane-1-carboxylic acid

### Synthetic Route

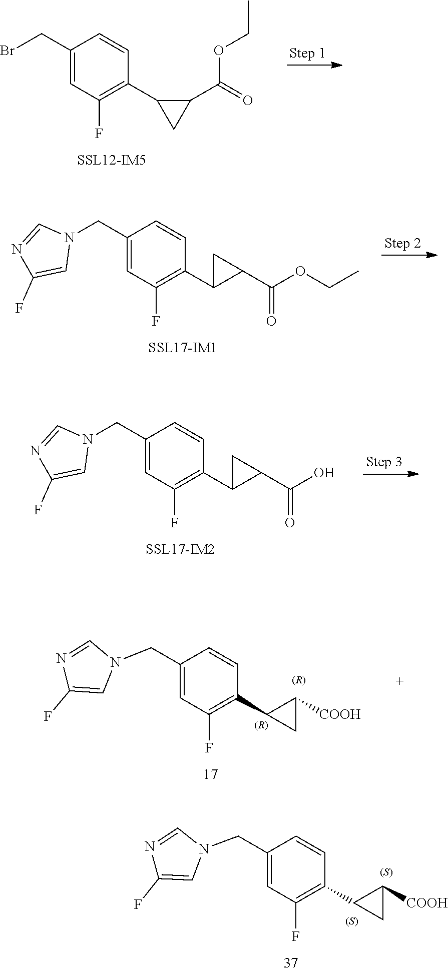

### Step 1

SSL12-IM5 (60 mg, 0.20 mmol, 1.0 eq), potassium carbonate (55 mg, 0.40 mmol, 2.0 eq), and 4-fluorimidazole (34 mg, 0.40 mmol, 2.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (45 mg).

### Step 2

SSL17-IM1 (45 mg, 0.14 mmol, 1.0 eq) was dissolved in tetrahydrofuran and methanol, then the mixture was added dropwise with lithium hydroxide aqueous solution (12 mg, 0.28 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (20 mg). ¹H NMR (400 MHz, Methanol-d₄) δ 7.41 (t, J=1.5 Hz, 1H), 7.15-6.92 (m, 3H), 6.69 (dd, J=7.9, 1.7 Hz, 1H), 5.12 (s, 2H), 2.60-2.47 (m, 1H), 1.93-1.73 (m, 1H), 1.50 (dt, J=9.4, 4.8 Hz, 1H), 1.33 (ddd, J=8.4, 6.3, 4.3 Hz, 1H). Mass: [M+H]⁺ 279.0.

### Step 3

SSL17-IM2 was obtained and purified by a chiral preparative column to obtain compound 17 and Compound 37.

### Example 18 Synthesis of Compound 18 and Compound 38

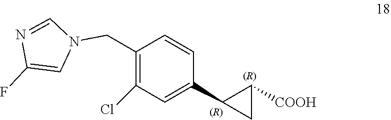

### (1R,2R)-2-(3-Chloro-4-((4-fluoro-1H-imidazol-1-yl)methyl)phenyl)cyclopropane-1-carboxylic acid

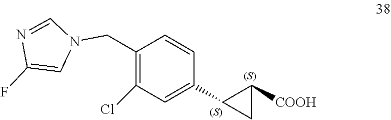

### (1S,2S)-2-(3-Chloro-4-((4-fluoro-1H-imidazol-1-yl)methyl)phenyl)cyclopropane-1-carboxylic acid

### Synthetic Route

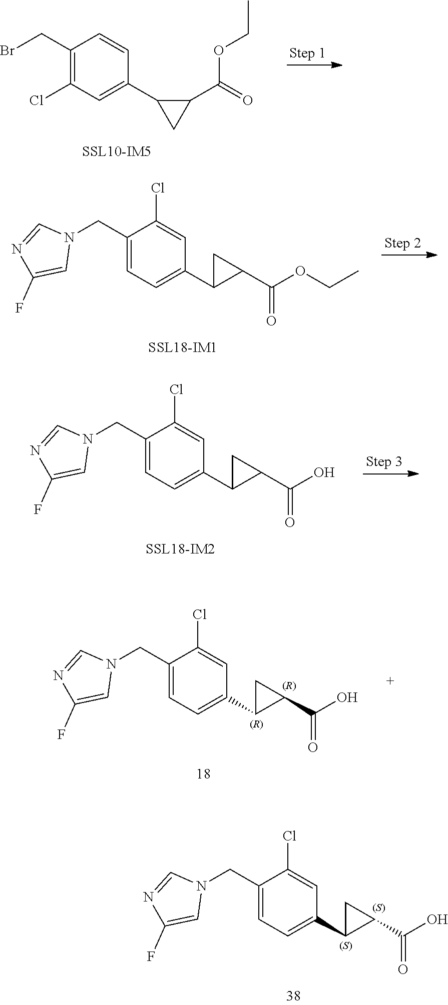

### Step 1

SSL10-IM5 (0.3 g, 0.945 mmol, 1.0 eq), potassium carbonate (0.52 g, 3.78 mmol, 4.0 eq), and 5-fluorimidazole (0.24 g, 2.83 mmol, 3.0 eq) were dissolved in acetonitrile, and then the reaction mixture was heated to 70° C. and stirred for 1 hour. The complete reaction of the raw material was monitored by TLC (PE/EA 6:1), then the reaction mixture was subjected to rotary evaporation until dryness, and the residue was subjected to column chromatography (DCM/CH₃OH 20:1) to obtain a solid (140 mg, yield of 46%).

### Step 2

SSL18-IM1 (0.14 g, 0.434 mmol, 1.0 eq) was dissolved in tetrahydrofuran and methanol, then the mixture was added dropwise with lithium hydroxide aqueous solution (0.037 g, 0.87 mmol, 2.0 eq) in an ice bath and stirred for 2 hours. The reaction mixture was subjected to rotary evaporation until dryness, and the residue was subjected to preparative thin-layer chromatography (DCM/CH₃OH 5:1) to obtain the product (111 mg, yield of 87%). ¹H NMR (400 MHz, MeOD) δ 7.37 (s, 1H), 7.20 (s, 1H), 7.15 (d, J=7.8 Hz, 1H), 7.08 (d, J=7.9 Hz, 1H), 6.67 (d, J=7.9 Hz, 1H), 5.22 (s, 2H), 2.32 (s, 1H), 1.75 (s, 1H), 1.46 (d, J=3.9 Hz, 1H), 1.14 (s, 1H). Mass: [M+H]⁺ 295.1.

### Step 3

SSL18-IM2 was obtained and purified by a chiral preparative column to obtain compound 18 and Compound 38.

Preparation method of a single crystal of compound 18: 3 mg of the target compound 18 was taken and placed in a 2.0 mL liquid phase vial, and 0.5 mL of CH₂Cl₂ was added thereto, then a solid suspension was seen. 3 to 4 drops of MeOH were added thereto, and the solid was dissolved. The vial was sealed with plastic wrap, and several small holes were pricked with a needle. The vial was placed in a 20 mL brown sample bottle containing 4.0 mL of n-hexane, and the brown sample bottle was sealed and placed in a refrigerator (2 to 8° C.) for 48 hours, and the crystal was observed to precipitate.

The structure data of a single crystal of compound 18 are as follows:

| Parameters | Single crystal of compound 18 |
|---|---|
| Experimental formula | C14H12ClFN2O2 |
| Molecular weight | 294.71 |
| Temperature | 113.15 K |
| Wavelength | 0.71073 A |
| Crystal system | Monoclinic |
| Space group | P 21 |
| Crystal cell size | a = 4.5886(2) Å, α = 90°. |
| | b = 10.9726(3) Å, β = 99.554(4)º. |
| | c = 13.3970(5) Å, γ = 90°. |
| Crystal cell volume | 665.17(4) Å³ |
| Z | 2 |
| Density (calculated) | 1.471 Mg/m³ |
| Absorption coefficient | 0.301 mm⁻¹ |
| F(000) | 304.0 |
| Crystal size | 0.23 × 0.2 × 0.17 mm³ |
| θ range for data collection | 4.826 to 65.816° |
| Range of indicators | −6 ≤ h ≤ 6, −16 ≤ k ≤ 16, −20 ≤ l ≤ 19 |
| Diffraction point collection | 9980 |
| Independent diffraction points | 4493 [Rint = 0.0310, Rsigma = 0.0400] |
| Refinement method | Goodness-of-fit on F² |

The single crystal structure of compound 18 is shown in FIG. 2.

### Example 19 Synthesis of Compound 19 and Compound 20

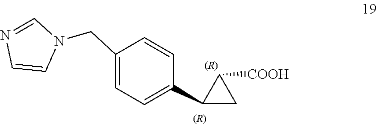

### (1R,2R)-2-(4-((1H-Imidazol-1-yl)methyl)phenyl)cyclopropane-1-carboxylic acid

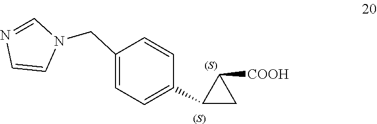

### (1S,2S)-2-(4-((1H-Imidazol-1-yl)methyl)phenyl)cyclopropane-1-carboxylic acid

### Synthetic Route

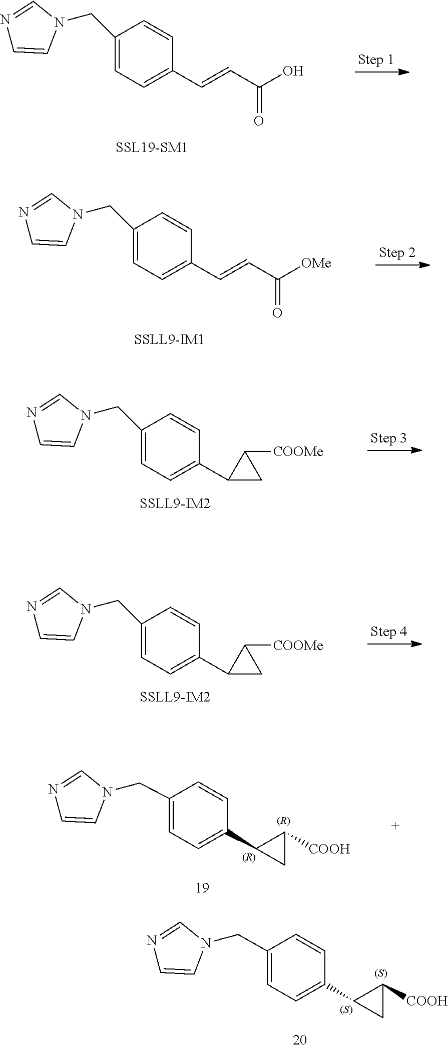

### Step 1

Methanol (132 mL) was measured and placed in a round-bottom flask, and concentrated sulfuric acid (33 mL) was slowly and added dropwise thereto while stirring in an ice bath, and then SSL19-SM1 (30 g, 132 mmol, 1.0 eq) was added thereto. The system was heated, refluxed, and reacted for 5 hours. After the reaction was complete, the reaction mixture was cooled to room temperature, poured into a large amount of crushed ice, and the pH was adjusted to 8 with saturated sodium bicarbonate solution. The mixture was extracted with ethyl acetate, and the organic phase was washed with saturated brine, dried, and concentrated under reduced pressure to obtain the white solid product (27.2 g, yield of 85%). ¹H NMR (400 MHz, Chloroform-d) δ 7.66 (d, J=16.0 Hz, 1H), 7.56 (s, 1H), 7.50 (d, J=8.1 Hz, 2H), 7.14 (s, 2H), 7.10 (s, 1H), 6.90 (s, 1H), 6.43 (d, J=16.0 Hz, 1H), 5.14 (s, 2H), 3.80 (s, 3H).

### Step 2

Trimethylsulfoxide iodide (15 g, 68.1 mmol, 1.1 eq) was weighed and added to a 250 mL reaction flask, and the reaction system was replaced with nitrogen, and then 65 mL of DMSO was added to dissolve the trimethylsulfoxide iodide. NaH (2.72 g, 68.1 mmol, 1.1 eq) was added thereto, and the reaction mixture was reacted for 1 hour at room temperature. SSL19-IM1 (15 g, 61.91 mmol, 1.0 eq) was dissolved in 65 mL of DMSO, slowly added dropwise to the system, and stirred for 2 to 3 hours at room temperature. After the reaction was complete, a large amount of saturated brine was added to quench the reaction, extracted five times with a small amount of ethyl acetate, and the organic phases were combined and dried. The organic phase was then concentrated under reduced pressure, and the residue was purified by column chromatography (ethyl acetate:methanol=30:1) to obtain the product (5 g, yield of 30%).

### Step 3

SSL19-IM2 (9.2 g, 35.89 mmol, 1.0 eq) was dissolved in tetrahydrofuran (110 mL) and methanol (55 mL), and then a solution of lithium hydroxide monohydrate (3 g, 71.78 mmol, 2.0 eq) in water (25 mL) was added dropwise thereto in an ice bath and stirred for 1 hour at room temperature. After the reaction was complete, the solvent was removed by rotary evaporation until dryness. The residue was added with methanol and filtered, and the filtrate was taken and subjected to rotary evaporation again until dryness. The residue was added with ethyl acetate, stirred, and filtered under reduced pressure to obtain the white solid product (8.28 g, yield of 95%). ¹H NMR (400 MHz, MeOD) δ 7.71 (s, 1H), 7.19-7.02 (m, 5H), 6.96 (s, 1H), 5.15 (s, 2H), 2.38-2.26 (m, 1H), 1.76-1.65 (m, 1H), 1.47-1.37 (m, 1H), 1.08 (ddd, J=8.5, 6.0, 4.2 Hz, 1H). Mass: [M+H]⁺ 243.1.

### Step 4

SSL19-IM3 was obtained and purified by a chiral preparative column to obtain compound 19 and Compound 20.

### Example 20 Synthesis of Compound 39

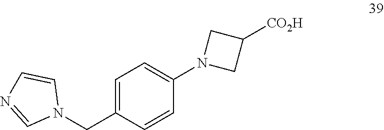 [SMILES: O=C(O)C1CN(c2ccc(Cn3ccnc3)cc2)C1]

### 1-(4-((1H-Imidazol-1-yl)methyl)phenyl)azetidine-3-carboxylic acid

### Synthetic Route

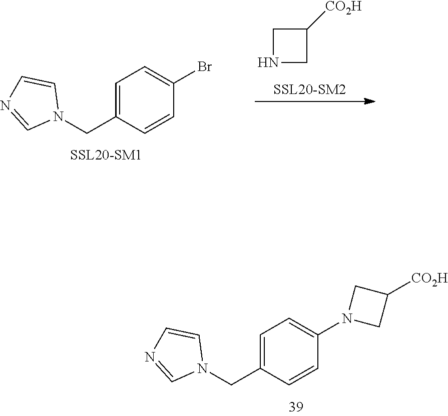 [SMILES: O=C(O)C1CNC1 ; Brc1ccc(Cn2ccnc2)cc1 ; O=C(O)C1CN(c2ccc(Cn3ccnc3)cc2)C1]

SSL20-SM1 (300 mg, 1.27 mmol, 1.00 eq), SSL20-SM2 (154 mg, 1.52 mmol, 1.20 eq), Pd(OAc)₂ (30.0 mg, 133 μmol, 0.105 eq), RuPhos (130 mg, 278 μmol, 0.22 eq), Cs₂CO₃ (1.65 g, 5.06 mmol, 4.00 eq), and t-BuOH (2.5 mL) were sequentially added to a 15 mL sealed tube under N₂ atmosphere. The reaction mixture was reacted for 16 hours at 90° C., and TLC (DCM/MeOH=10/1, R_{f}=0.05) was used to monitor the reaction until the reaction was complete. The pH of the reaction mixture was adjusted to 2 with TFA, and the reaction mixture was concentrated to remove the solvent and subjected to column chromatography (DCM/MeOH=20/1 to 10/1) and prep-TLC to obtain a light yellow solid (82 mg). ¹H NMR (400 MHz, DMSO) δ 13.63-11.92 (m, 1H), 8.04 (s, 1H), 7.26 (s, 1H), 7.16 (d, J=8.3 Hz, 2H), 7.04 (s, 1H), 6.43 (d, J=8.3 Hz, 2H), 5.08 (s, 2H), 3.98 (t, J=7.9 Hz, 2H), 3.82 (t, J=6.6 Hz, 2H), 3.56-3.46 (m, 2H). Mass: [M+H]⁺ 258.0.

### Example 21 Synthesis of Compound 40

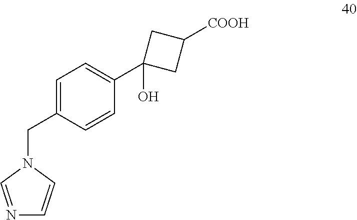 [SMILES: O=C(O)C1CC(O)(c2ccc(Cn3ccnc3)cc2)C1]

### 3-(4-((1H-Imidazol-1-yl)methyl)phenyl)-3-hydroxycyclobutane-1-carboxylic acid

### Synthetic Route

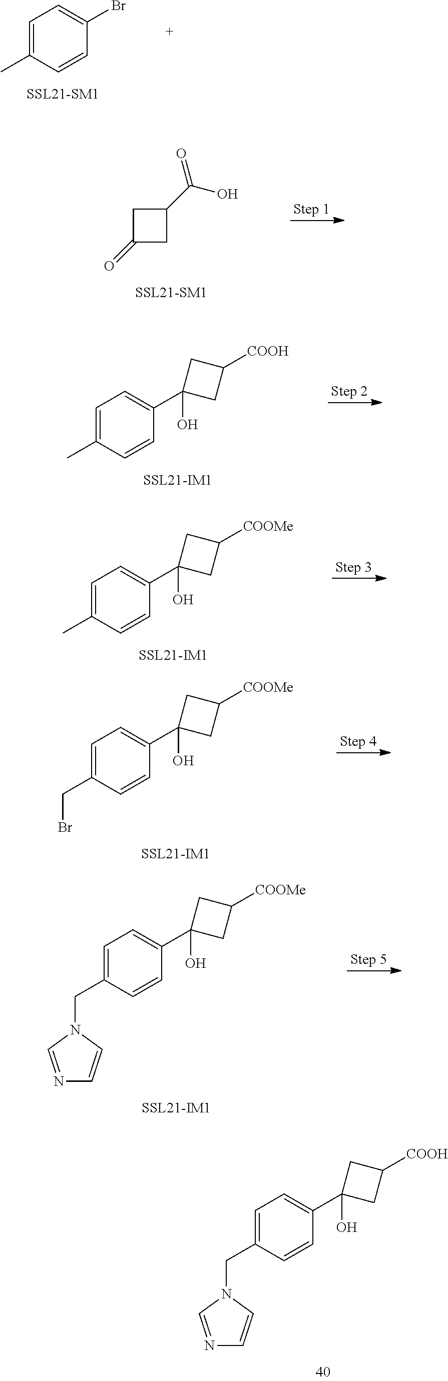

### Step 1

A250 mL dry round-bottom flask was taken, and p-bromotoluene SSL21-SM1 (4.5 g, 26.29 mmol, 2.0 eq) was added thereto, dissolved in 50 mL of THF, and placed at −78° C. for 10 minutes, after which n-BuLi (11.0 mL, 0.54 mmol, 2.0 eq) was slowly added thereto, and solid precipitation was noted in the process of the addition. A turbid mixture was obtained and stirred for 2.0 hours at this temperature. Another 50 mL reaction flask was taken, the substrate SSL21-SM2 (1.5 g, 13.15 mmol, 1.0 eq) was added thereto, added with toluene and concentrated for three times to remove water, dissolved with 10 mL of dry THF, which was slowly added to the turbid mixture and washed twice with another 5 mL of THF, stirred for 10 minutes at this temperature, and then reacted for 0.5 hours at room temperature. The reaction was quenched with 50 mL of a 10% NaHSO₄ solution and extracted with EA. The organic phases were combined, concentrated, and used directly in the next step.

### Step 2

The crude product of SSL21-IM1 was taken and dissolved in 50 mL of acetone, and K₂CO₃ (3.6 g, 26.29 mmol, 2.0 eq) and Mel (1.6 mL, 26.29 mmol, 2.0 eq) were added thereto, and the reaction mixture was reacted overnight at 60° C. TLC (PE:EA=3:1) was used to monitor the reaction to obtain the main product point. The reaction mixture was concentrated and purified by column chromatography (PE:EA=4:1) to obtain the oily product (1.4 g, yield of 50%).

### Step 3

Compound SSL21-IM2 (500 mg, 2.27 mmol, 1.0 eq) was taken and dissolved in 20 mL of CCl₄, and NBS (444 mg, 2.50 mmol, 1.1 eq) and AIBN (38 mg, 0.23 mmol, 0.1 eq) were added thereto, and then the reaction mixture was replaced three times with nitrogen and reacted overnight at 70° C. TLC (PE:EA=3:1) was used to monitor the reaction until the starting material disappeared completely. The reaction mixture was concentrated and purified by column chromatography (PE:EA=3:1) to obtain the yellow solid product (490 mg, yield of 72%).

### Step 4

Compound SSL21-IM3 (150 mg, 0.50 mmol, 1.0 eq) was taken and dissolved in 5.0 mL of CH₃CN, and K₂CO₃ (207 mg, 1.50 mmol, 3.0 eq) and imidazole (102 mg, 1.5 mmol, 3.0 eq) were added thereto, and then the reaction mixture was replaced three times with nitrogen and reacted for 5.0 hours at 60° C. The generation of the product was detected by TLC (DCM:MeOH=10:1). The reaction mixture was concentrated and subjected to PTLC (DCM:MeOH=10:1) to obtain the white solid product (69 mg, yield of 45%).

### Step 5

Compound SSL21-IM4 (30 mg, 0.11 mmol, 1.0 eq) was taken, and 0.3 mL of THF and 0.3 mL of MeOH were added thereto to dissolved compound SSL21-IM4, and then 0.3 mL of H₂O and LiOH (9 mg, 0.21 mmol, 2.0 eq) were added thereto, and the reaction mixture was reacted for 3.0 hours at room temperature. TLC (DCM:MeOH=10:1) was used to monitor the reaction until the starting material disappeared completely, then the reaction was quenched by the addition of 1.0 M HCl (200 μL, 0.2 mmol, 2.0 eq), and filtered through silica gel under reduced pressure to obtain the target compound. 1H NMR (400 MHz, Methanol-d4) δ 7.74 (s, 1H), 7.54 (d, J=8.3 Hz, 2H), 7.26 (d, J=8.3 Hz, 2H), 7.10 (s, 1H), 6.97 (s, 1H), 5.21 (s, 2H), 2.77-2.69 (m, 2H), 2.65-2.59 (m, 1H), 2.57-2.50 (m, 2H). Mass: [M+H]⁺ 273.1.

### Example 22 Synthesis of Compound 41

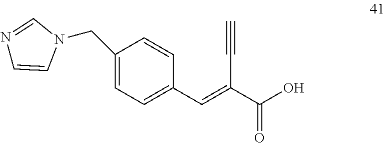 [SMILES: C#C/C(=C\c1ccc(Cn2ccnc2)cc1)C(=O)O]

### (E)-2-(4-((1H-Imidazol-1-yl)methyl)benzylidene)butynoic acid

### Synthetic Route

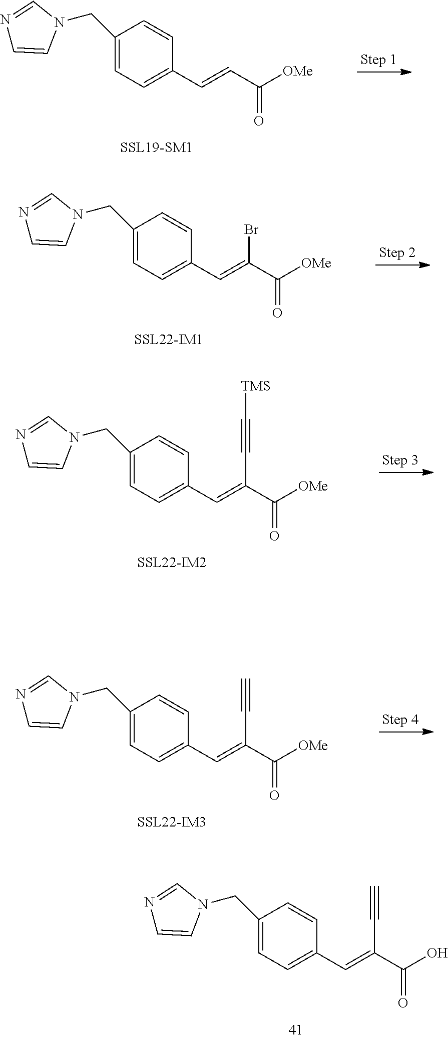 [SMILES: COC(=O)/C=C/c1ccc(Cn2ccnc2)cc1 ; COC(=O)/C(Br)=C/c1ccc(Cn2ccnc2)cc1 ; COC(=O)/C(C#C[Si](C)(C)C)=C/c1ccc(Cn2ccnc2)cc1 ; C#C/C(=C\c1ccc(Cn2ccnc2)cc1)C(=O)OC ; C#C/C(=C\c1ccc(Cn2ccnc2)cc1)C(=O)O]

### Step 1

DMP (12.6 g, 5.95 mmol, 1.20 eq) was dissolved in DCM (150 mL), and the mixture was added with TBAB (9.60 g, 5.95 mmol, 1.20 eq) under nitrogen atmosphere, stirred for 30 minutes until the solution turned to orange, added with SSL19-IM1 (6.00 g, 4.94 mmol, 1.00 eq), and stirred for 24 hours at room temperature until the reaction monitored by TLC (DCM/MeOH=25/1, R_{f}=0.6, polarity decreased) was complete. The reaction mixture was sequentially washed with 10% Na₂S₂O₃, saturated NaHCO₃, and saturated brine, and the organic phase was dried over anhydrous sodium sulfate, concentrated to remove the solvent, and purified by column chromatography (PE/EA/DCM=3/1/1 to 2/1/1) to obtain the crude product, which was slurried and subjected to preparative thin-layer chromatography to obtain a white solid (1.20 g, yield of 15%). ¹H NMR (400 MHz, Chloroform-d) δ 7.67 (d, J=16.1 Hz, 1H), 7.61 (s, 1H), 7.52 (d, J=8.3 Hz, 2H), 7.15 (d, J=8.1 Hz, 2H), 7.09 (s, 1H), 6.44 (d, J=16.0 Hz, 1H), 5.16 (s, 2H), 3.82 (s, 3H).

### Step 2

Pd(PPh₃)₄ (72.0 mg, 62.4 μmol, 0.100 eq), CuI (24.0 mg, 124 μmol, 0.200 eq), TEA (320 μL, 1.87 mmol, 3.00 eq), and THE (2.00 mL) were sequentially added to a 25 mL round-bottom flask under nitrogen atmosphere. SSL22-IM1 (200 mg, 624 μmol, 1.00 eq) was dissolved in THE (1.00 mL), which was added to the reaction mixture, and bubbled with nitrogen for 10 minutes. The reaction mixture was added with trimethylsilylacetylene (240 L, 1.87 mmol, 3.00 eq) and stirred for 24 hours at 70° C. until the reaction monitored by TLC (PE/EA=½, R_{f}=0.3, polarity slightly decreased) was complete. The reaction mixture was directly subjected to rotary evaporation until dryness and purified by column chromatography (PE/EA=5/1 to 1/1) to obtain a brown oily liquid (170 mg, yield of 81%).

### Step 3

TBAF (500 μL, 0.500 mmol, 1.00 eq) was added dropwise to a solution of SSL22-IM2 (170 mg, 0.500 mmol, 1.00 eq) in THE (1.50 mL) at 0° C. under N₂ atmosphere. The reaction mixture was reacted for 1 hour at 0° C. until the reaction monitored by TLC (PE/EA=1/1, R_{f}=0.6, polarity slightly increased) was complete. The reaction mixture was diluted with 10 mL of water, extracted with ethyl acetate (5 mL×3), and washed with saturated brine, and the organic phases were dried over anhydrous sodium sulfate, concentrated to remove the solvent, and subjected to preparative thin-layer chromatography to obtain a yellow solid (93 mg, yield of 69%).

### Step 4

LiOH H₂O (17.6 mg, 419 μmol, 1.20 eq) was added to a solution of SSL22-IM3 (93.0 mg, 349 μmol, 1.00 eq) in THF-MeOH—H₂O (0.4 mL+0.4 mL+0.2 mL), and the reaction mixture was stirred for 14 hours at room temperature until the reaction monitored by TLC (DCM/MeOH=10/1, R_{f}=0.1) was complete. The reaction mixture was diluted with water (10 mL), and the pH was adjusted to 2 to 3 with iN HCl, extracted with DCM, concentrated, and purified by prep-HPLC to obtain a white solid (40 mg, yield of 45%). ¹H NMR (400 MHz, Methanol-d₄) δ 7.85-7.55 (m, 5H), 7.40 (d, J=8.0 Hz, 2H), 6.52 (d, J=16.0 Hz, 1H), 5.50 (s, 2H), 4.36 (s, 1H). Mass: [M+H]⁺ 253.0.

### Example 23 Synthesis of Compound 42

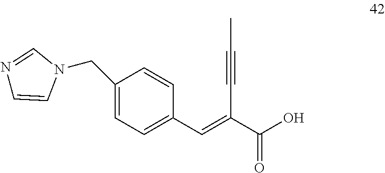 [SMILES: CC#C/C(=C\c1ccc(Cn2ccnc2)cc1)C(=O)O]

### (E)-2-(4-((1H-Imidazol-1-yl)methyl)benzylidene)pent-3-ynoic acid

### Synthetic Route

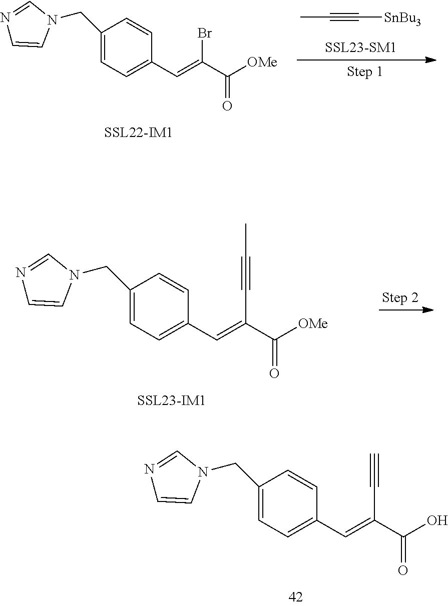

### Step 1

Pd(PPh₃)₄ (55.0 mg, 0.048 mmol, 0.3 eq), SSL22-IM1 (50 mg, 0.16 mmol, 1.0 eq), SSL23-SM1 (0.1 mL, 0.32 mmol, 2.0 eq), and PhMe (2.00 mL) were sequentially added to a 25 mL round-bottom flask under nitrogen atmosphere. The reaction mixture was stirred for 12 hours at 100° C. until the reaction monitored by TLC (DCM/MeOH=20:1, R_{f}=0.3, polarity slightly decreased) was complete. The reaction mixture was directly subjected to rotary evaporation until dryness and then purified by preparative thin-layer chromatography to obtain the product.

### Step 2

SSL23-IM1 (50 mg, 0.17 mmol, 1.0 eq) was dissolved in tetrahydrofuran and methanol, then the mixture was added dropwise with lithium hydroxide aqueous solution (15 mg, 0.34 mmol, 2.0 eq) in an ice bath and stirred for 2 hours. The reaction mixture was subjected to rotary evaporation until dryness, and the residue was subjected to preparative thin-layer chromatography (DCM/CH₃OH 15:1) to obtain the product (20 mg, yield of 44%). 1H NMR (400 MHz, Methanol-d4) δ 7.74 (s, 1H), 7.53 (d, J=8.2 Hz, 2H), 7.44 (d, J=15.9 Hz, 1H), 7.23 (d, J=8.0 Hz, 2H), 7.08 (s, 1H), 6.49 (d, J=16.0 Hz, 1H), 5.24 (s, 2H), 2.04 (s, 3H).

Mass: [M+H]⁺ 267.1.

### Example 24 Synthesis of Compound 43

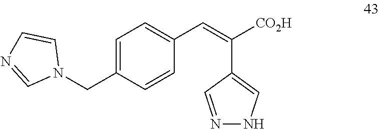 [SMILES: O=C(O)/C(=C/c1ccc(Cn2ccnc2)cc1)c1cn[nH]c1]

### (E)-3-(4-((1H-Imidazol-1-yl)methyl)phenyl)-2-(1H-pyrazol-4-yl)acrylic acid

### Synthetic Route

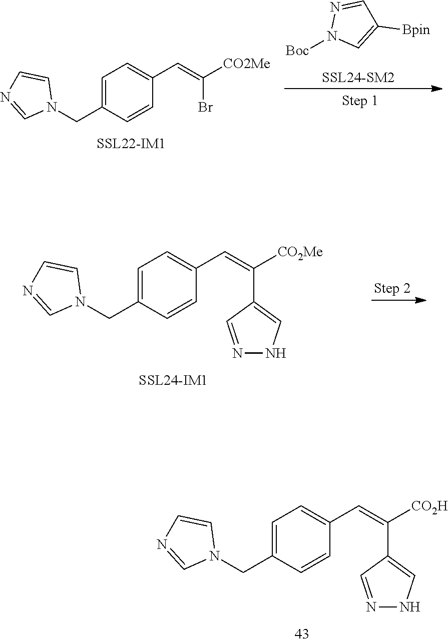

### Step 1

SSL22-IM1 (300 mg, 936 μmol, 1.00 eq), SSL24-SM2 (420 mg, 1.40 mmol, 1.50 eq), Pd(tBu₃P)₂ (96.0 mg, 187 μmol, 0.20 eq), Cs₂CO₃ (612 mg, 1.87 mmol, 2.00 eq), and iPrOH-H₂O (2.4 mL+0.6 mL) were sequentially added to a 20 mL tube with side neck under nitrogen atmosphere. The reaction mixture was reacted at 90° C. for 12 hours until the reaction monitored by TLC (DCM/MeOH=10:1, R_{f}=0.5, polarity increased, another point at R_{f}=0.4) was complete. The reaction mixture was dried over anhydrous magnesium sulfate, concentrated to remove the solvent, and purified by column chromatography (DCM/MeOH=100/1 to 20/1) to obtain a yellow oil (90 mg, yield of 68%).

¹H NMR (400 MHz, MeOD) δ 7.83 (s, 1H), 7.59 (d, J=14.9 Hz, 2H), 7.49 (d, J=7.9 Hz, 1H), 7.10 (s, 1H), 7.02 (d, J=7.9 Hz, 1H), 6.45 (d, J=16.0 Hz, 1H), 5.29 (s, 1H), 3.73 (s, 2H).

### Step 2

Compound SSL24-IM1 (20 mg, 0.065 mmol, 1.0 eq) was taken, and 0.5 mL of THF and 0.5 mL of MeOH were added thereto to dissolved compound SSL24-IM1, and then the mixture was added with 0.5 mL of H₂O and LiOH (10 mg, 0.26 mmol, 4.0 eq), and reacted for 0.5 hours at 50° C. until the reaction monitored by TLC was complete. The pH of the mixture was adjusted to 4 by the addition of 1.0 M HCl to obtain 15 mg of the product. ¹H NMR (400 MHz, MeOH-d₄) δ 9.27 (s, 1H), 7.96 (s, 2H), 7.77 (s, 1H), 7.70-7.51 (m, 5H), 7.21 (d, J=7.6 Hz, 2H), 6.47 (d, J=16.0 Hz, 1H), 5.63 (s, 2H). Mass: [M+H]⁺ 295.1.

### Example 25 Synthesis of Compound 44

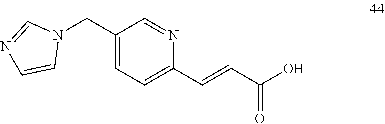 [SMILES: O=C(O)/C=C/c1ccc(Cn2ccnc2)cn1]

### (E)-3-(5-((1H-Imidazol-1-yl)methyl)pyridin-2-yl)acrylic acid

### Synthetic Route

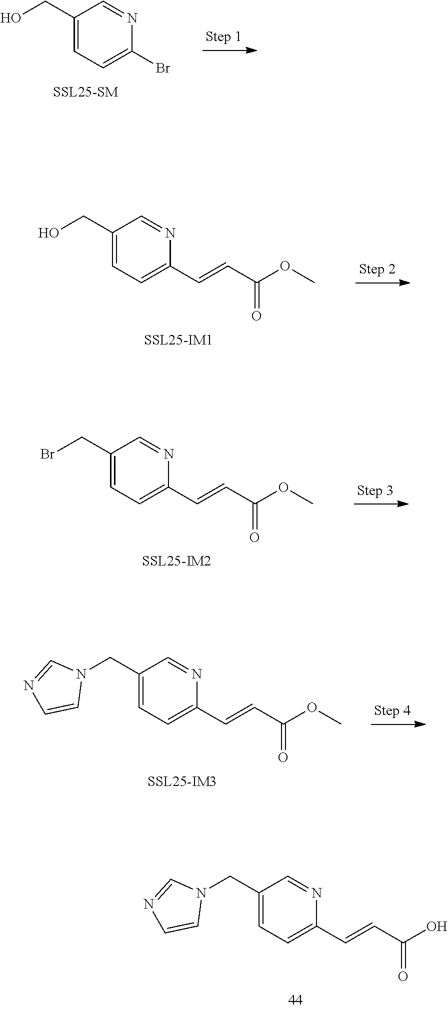 [SMILES: OCc1ccc(Br)nc1 ; COC(=O)/C=C/c1ccc(CO)cn1 ; COC(=O)/C=C/c1ccc(CBr)cn1 ; COC(=O)/C=C/c1ccc(Cn2ccnc2)cn1 ; O=C(O)/C=C/c1ccc(Cn2ccnc2)cn1]

### Step 1

6-Bromo-3-pyridinemethanol SSL25-SM (3.0 g, 0.016 mol, 1.0 eq), palladium acetate (0.36 g, 0.0016 mol, 0.1 eq), and POT (0.97 g, 0.0032 mol, 0.2 eq) were dissolved in DMF in a 75 mL sealed tube, and TEA (6.65 mL, 0.048 mol, 3.0 eq) and methyl acrylate (13.78 g, 0.16 mol, 10.0 eq) were added thereto. The reaction mixture was heated to 110° C. and stirred overnight, and the complete reaction of the raw material was monitored by TLC (PE/EA=1:1). The reaction mixture was poured into 100 mL of ice water, extracted by EA (20 mL*5), and the organic phases were combined, then washed with saturated sodium chloride, dried, and subjected to rotary evaporation until dryness. The residue was subjected to column chromatography (PE:EA=3:1) to obtain a light yellow solid (900 mg, yield of 29%).

### Step 2

SSL25-IM1 (900 mg, 0.0047 mol, 1.0 eq) was dissolved in 10 mL of dichloromethane, and phosphorus tribromide (5.0 g, 0.0188 mol, 4 eq) was added dropwise under an ice bath, warmed to room temperature, and stirred for 30 minutes. After the reaction was completed, the reaction mixture was poured into ice water and added with sodium carbonate solid. The pH of the mixture was adjusted to 9, and the mixture was extracted with ethyl acetate, dried over anhydrous sodium sulfate, subjected to rotary evaporation until dryness, and the residue was subjected to column chromatography (PE:EA=5:1) to obtain a white solid (400 mg, yield of 33%). ¹H-NMR (400 MHz, DMSO) δ 8.72 (s, 1H), 7.93-7.95 (m, 1H), 7.76 (d, J=8.0 Hz, 2H), 7.67 (d, J=16.0 Hz, 1H), 6.91 (d, J=16.0 Hz, 1H), 4.78 (s, 3H), 3.75 (s, 3H).

### Step 3

SSL25-IM2 (400.0 mg, 1.56 mmol, 1.0 eq), potassium carbonate (432.7 mg, 3.12 mmol, 2.0 eq), and imidazole (106.7 mg, 1.56 mmol, 1.0 eq) were dissolved in acetonitrile, and then the reaction mixture was heated to 60° C. and stirred for 2 hours. The complete reaction of the raw material was monitored by TLC (DCM:CH₃OH=20:1), then the reaction mixture was subjected to rotary evaporation until dryness, and the residue was subjected to column chromatography (DCM:CH₃OH=20:1) to obtain a white solid (270 mg, yield of 70%).

### Step 4

SSL25-IM3 (270 mg, 1.1 mmol, 1.0 eq) was dissolved in tetrahydrofuran and methanol, and then a solution of lithium hydroxide monohydrate (94 mg, 2.23 mmol, 2.0 eq) in water was added dropwise thereto in an ice bath, stirred for 1 hour, subjected to rotary evaporation until dryness, added with 10 mL of methanol, filtered to obtain the filtrate. The filtrate was subjected to rotary evaporation until dryness, added with 10 mL of ethyl acetate, stirred, and filtered under reduced pressure to obtain a white solid (220 mg, yield of 86%). ¹H-NMR (400 MHz, DMSO) δ 9.42 (s, 1H), 8.79-8.81 (m, 1H), 8.02-8.04 (m, 1H), 7.86-7.90 (m, 1H), 7.68-7.72 (m, 1H), 7.60-7.64 (m, 1H), 7.63 (d, J=15.6 Hz, 1H), 6.92 (d, J=8.0 Hz, 1H), 5.58 (s, 2H). Mass: [M+H]⁺ 230.1.

### Example 26 Synthesis of Compound 45

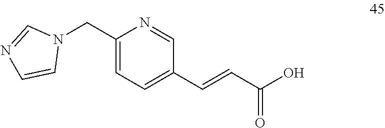 [SMILES: O=C(O)/C=C/c1ccc(Cn2ccnc2)nc1]

### (E)-3-(6-((1H-Imidazol-1-yl)methyl)pyridin-3-yl)acrylic acid

### Synthetic Route

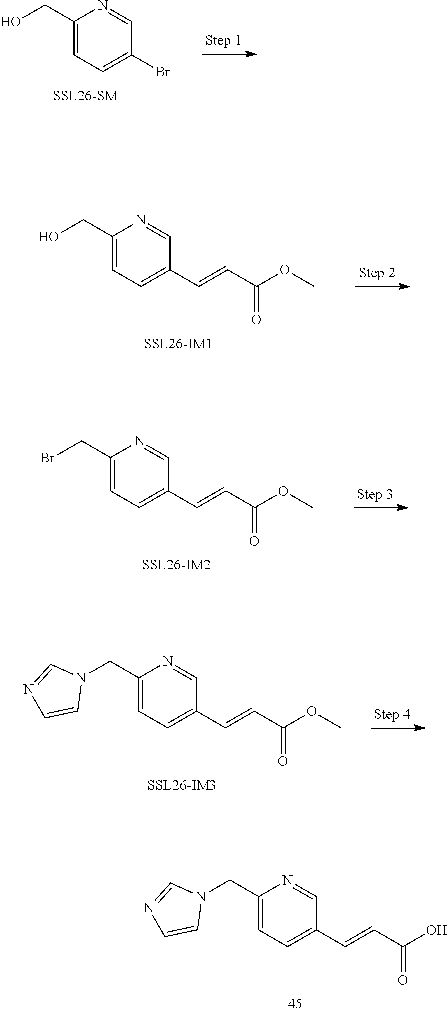 [SMILES: OCc1ccc(Br)cn1 ; COC(=O)/C=C/c1ccc(CO)nc1 ; COC(=O)/C=C/c1ccc(CBr)nc1 ; COC(=O)/C=C/c1ccc(Cn2ccnc2)nc1 ; O=C(O)/C=C/c1ccc(Cn2ccnc2)nc1]

### Step 1

5-Bromo-2-pyridinemethanol SSL26-SM (4.5 g, 0.024 mol, 1.0 eq), palladium acetate (0.54 g, 0.0024 mol, 1.0 eq), and POT (1.47 g, 0.0048 mol, 0.2 eq) were dissolved in DMF, and the mixture was replaced three times with nitrogen, added with TEA (7.34 g, 0.072 mol, 3.0 eq) and methyl acrylate (20.83 g, 0.24 mol, 10.0 eq), heated to 80° C., and stirred for 2 hours. The complete reaction of the raw material was monitored by TLC (PE:EA=1:3). The reaction mixture was poured into 100 mL of ice water, extracted by EA (20 mL*5), and the organic phases were combined, then washed with saturated sodium chloride, dried, subjected to rotary evaporation until dryness, then slurried with 50 mL of TBME, and filtered under reduced pressure to obtain a light yellow solid (2.7 g, yield of 58%).

### Step 2

SSL26-IM1 (2.7 g, 0.014 mol, 1.0 eq) was dissolved in 30 mL of dichloromethane, and then the mixture was added with tetrabromomethane (6.0 g, 0.018 mol, 1.3 eq), maintained at 0° C., added with triphenylphosphine (4.42 g, 0.016 mmol, 1.2 eq) in batches, still maintained at 0° C., and the reaction mixture was stirred for 2 hours at 0° C. After the reaction was complete, the reaction mixture was subjected to rotary evaporation until dryness, and the residue was subjected to column chromatography (PE:EA=5:1) to obtain a light yellow solid (2.3 g, yield of 65%).

### Step 3

SSL26-IM2 (2.3 g, 0.009 mol, 1.0 eq), potassium carbonate (2.48 g, 0.018 mol, 2.0 eq), and imidazole (0.61 g, 0.0090 mol, 1.0 eq) were dissolved in acetonitrile, and then the reaction mixture was heated to 80° C. and stirred for 1 hour. The complete reaction of the raw material was monitored by TLC (DCM:CH₃OH=20:1), then the reaction mixture was subjected to rotary evaporation until dryness, and the residue was subjected to column chromatography (EA:PE=9:1 to DCM:CH₃OH=20:1) to obtain a white solid (0.8 g, yield of 38%).

### Step 4

SSL26-IM3 (0.8 g, 0.0033 mol, 1.0 eq) was dissolved in tetrahydrofuran and methanol, and then a solution of lithium hydroxide monohydrate (0.27 g, 0.0066 mol, 2.0 eq) in water was added dropwise thereto in an ice bath, stirred for 1 hour, subjected to rotary evaporation until dryness, added with 10 mL of methanol, filtered to obtain the filtrate. The filtrate was subjected to rotary evaporation until dryness, added with 10 mL of ethyl acetate, stirred, and filtered under reduced pressure to obtain a white solid (0.72 g, yield of 96%). ¹H-NMR (400 MHz, DMSO) δ 9.42 (s, 1H), 8.45 (m, 1H), 8.30 (m, 1H), 7.83 (m, 1H), 7.70 (m, 1H), 7.62 (m, 1H), 7.60 (d, J=10.0 Hz, 1H), 6.73 (d, J=10.0 Hz, 1H), 5.73 (s, 1H). Mass: [M+H]⁺ 230.1.

### Example 27 Synthesis of Compound 46

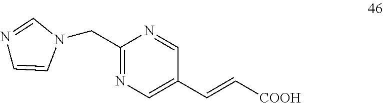 [SMILES: O=C(O)/C=C/c1cnc(Cn2ccnc2)nc1]

### (E)-3-(2-((1H-Imidazol-1-yl)methyl)pyrimidin-5-yl)acrylic acid

### Synthetic Route

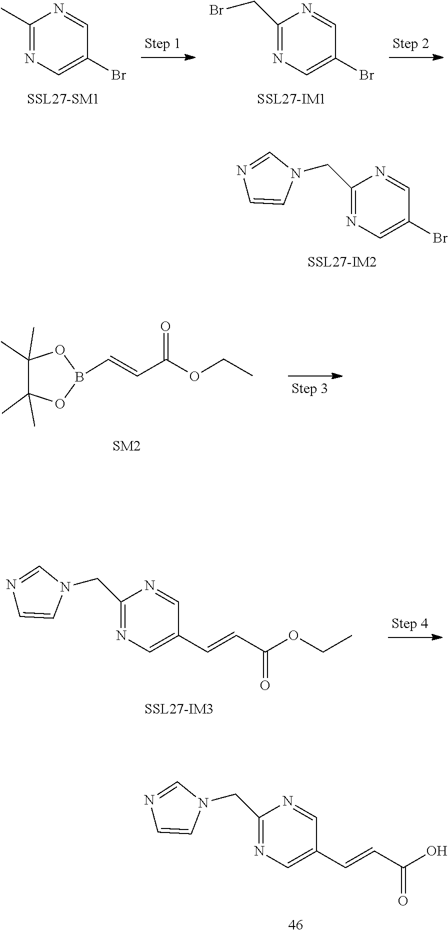

### Step 1

SSL27-SM1 (1 g, 7.30 mmol, 1.0 eq), NBS (1.29 g, 10.95 mmol, 1.5 eq), and AIBN (119 mg, 0.73 mmol, 0.1 eq) were sequentially added to a 50 mL round-bottom flask, and the reaction mixture was replaced three times with nitrogen, then injected with 11 mL of CCl₄, and the system was placed in an oil bath at 70° C. and reacted overnight. TLC was used to monitor the reaction until the reaction was complete, and the reaction mixture was concentrated and used directly in the next step.

### Step 2

SSL27-IM/1 (3.65 mmol, 1.0 eq), Cs₂CO₃ (2.378 g, 7.30 mmol, 2.0 eq), and imidazole (478 mg, 7.96 mmol, 2.2 eq) were sequentially added to a 25 mL round-bottom flask, then injected with 3 mL of acetone, and the reaction mixture was stirred at room temperature for 1 hour. TLC was used to monitor the reaction until the reaction was complete, and the reaction mixture was filtered by diatomite, and separated by preparative thin-layer chromatography to obtain the product (800 mg, a two-step yield of 58%).

### Step 3

10 mL of THE and 2 mL of H₂O were added to a 25 mL round-bottom flask, and bubbled with nitrogen for 30 minutes to remove oxygen. Another 25 mL round-bottom flask was taken, and a magnetic stirrer, SSL27-IM2 (210 mg, 0.87 mmol, 1.0 eq), K₂CO₃ (600 mg, 4.35 mmol, 5.0 eq), Pd(dppf)Cl₂ (129 mg, 0.174 mmol, 0.2 eq), and SM2 (0.3 mL, 1.305 mol, 1.5 eq) were sequentially added thereto. A reflux tube was installed, the system was replaced 5 times with nitrogen, and 5.3 mL of a mixture of THF and H₂O (5:1) was added thereto, and the reaction mixture was placed in a 95° C. oil bath and refluxed overnight. TLC was used to monitor the reaction until a small amount of starting material remained, and the reaction mixture was filtered by diatomite, subjected to rotary evaporation until dryness, and subjected to preparative thin-layer chromatography to obtain the product (105 mg, yield of 46%).

### Step 4

SSL27-IM3 (105 mg, 0.41 mmol, 1.0 eq) was dissolved in tetrahydrofuran and methanol, and then a solution of lithium hydroxide monohydrate (34.4 mg, 0.82 mmol, 2.0 eq) in water was added dropwise thereto in an ice bath, stirred for 1 hour, subjected to rotary evaporation until dryness, added with 2 mL of methanol, and filtered to obtain the filtrate, and the filtrate was subjected to rotary evaporation until dryness, added with 2 mL of ethyl acetate, stirred, and filtered under reduced pressure to obtain the gray solid product (90 mg, yield of 97%). ¹H NMR (400 MHz, Methanol-d4) δ 8.89 (s, 1H), 7.95-7.69 (m, 1H), 7.33 (d, J=6.4 Hz, 1H), 7.19 (s, 1H), 6.99 (s, 1H), 6.81-6.52 (d, J=16 Hz 1H), 5.45 (s, 2H). Mass: [M+H]⁺ 231.1.

### Example 28 Synthesis of Compound 47

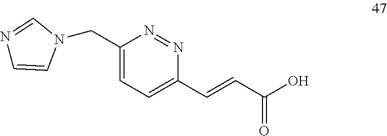 [SMILES: O=C(O)/C=C/c1ccc(Cn2ccnc2)nn1]

### (E)-3-(6-((1H-Imidazol-1-yl)methyl)pyridazin-3-yl)acrylic acid

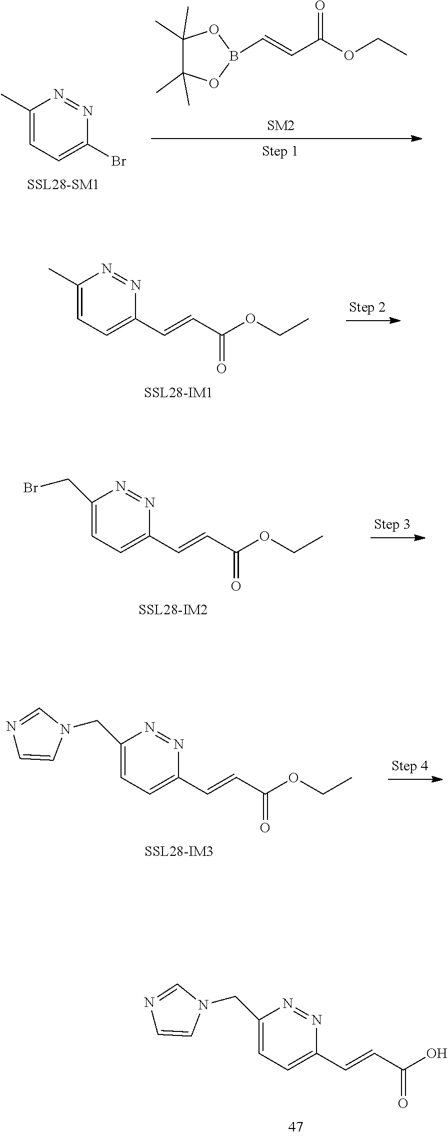

### Step 1

6 mL of DMF and 2 mL of H₂O were added to a 10 mL round-bottom flask, and bubbled with nitrogen for 30 minutes to remove oxygen. A magnetic stirrer, SSL28-SM1 (10 mg, 0.06 mmol, 1.0 eq), K₂CO₃ (16 mg, 0.12 mmol, 2.0 eq), Pd(dppf)Cl₂ (8.7 mg, 0.012 mmol, 0.2 eq), and SM2 (24 mg, 0.12 mmol, 2.0 eq) were sequentially added to a 5 mL round-bottom flask. The system was replaced 5 times with nitrogen, and 0.25 mL of a mixture of DMF:H₂O (3:1) was injected thereto, and the reaction mixture was placed in an 80° C. oil bath and reacted for 3 hours. The complete reaction of the raw material was monitored by TLC, and the reaction mixture was filtered by diatomite and subjected to rotary evaporation until dryness. The product was used directly in the next step without purification.

### Step 2

SSL28-IM1 (0.06 mmol, 1.0 eq), NBS (10.6 mg, 0.09 mmol, 1.5 eq), and AIBN (1 mg, 0.006 mmol, 0.1 eq) were sequentially added to a 5 mL round-bottom flask, and the system was replaced three times with nitrogen, then injected with 0.25 mL of CCl₄, and the system was placed in a 60° C. oil bath and reacted for 4 hours. TLC was used to monitor the reaction until the reaction was complete, and the reaction mixture was used directly in the next step without purification.

### Step 3

SSL28-IM2 (0.03 mmol, 1.0 eq), Cs₂CO₃ (20 mg, 0.06 mmol, 2.0 eq), and imidazole (4 mg, 0.066 mmol, 2.2 eq) were sequentially added to a 5 mL round-bottom flask, then injected with 0.25 mL of acetone, and the reaction mixture was stirred at room temperature overnight. TLC was used to monitor the reaction until the reaction was complete, and the reaction mixture was filtered by diatomite, and separated by preparative thin-layer chromatography to obtain the product. 1H NMR (400 MHz, Chloroform-d) δ 7.82 (d, J=16.0 Hz, 1H), 7.67 (s, 1H), 7.63 (s, 1H), 7.57 (d, J=8.8 Hz, 1H), 7.14 (s, 1H), 7.10 (d, J=8.5 Hz, 2H), 5.51 (s, 2H), 4.29 (q, J=7.0 Hz, 2H), 1.34 (t, J=7.1 Hz, 3H).

### Step 4

SSL28-IM3 (6 mg, 0.023 mmol, 1.0 eq) was dissolved in tetrahydrofuran and methanol, and then a solution of lithium hydroxide monohydrate (2 mg, 0.046 mmol, 2.0 eq) in water was added dropwise thereto in an ice bath, stirred for 1 hour, subjected to rotary evaporation until dryness, added with 2 mL of methanol, and filtered to obtain the filtrate, and the filtrate was subjected to rotary evaporation until dryness. ¹H NMR (400 MHz, Methanol-d4) δ 7.71 (d, J=8.8 Hz, 1H), 7.61 (s, 1H), 7.41 (s, 1H), 7.32 (s, 1H), 7.29 (s, 1H), 6.99-6.95 (m, 1H), 6.72 (d, J=16.1 Hz, 1H), 4.87 (s, 2H).

### Example 29 Synthesis of Compound 48

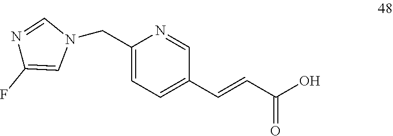 [SMILES: O=C(O)/C=C/c1ccc(Cn2cnc(F)c2)nc1]

### (E)-3-(6-((4-Fluoro-1H-imidazol-1-yl)methyl)pyridin-3-yl)acrylic acid

### Synthetic Route

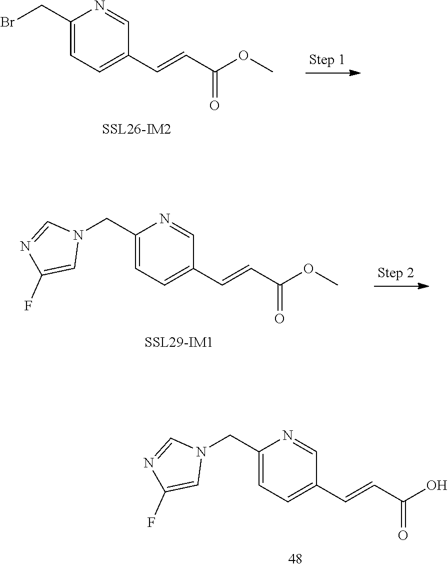 [SMILES: COC(=O)/C=C/c1ccc(CBr)nc1 ; COC(=O)/C=C/c1ccc(Cn2cnc(F)c2)nc1 ; O=C(O)/C=C/c1ccc(Cn2cnc(F)c2)nc1]

### Step 1

5-Fluoroimidazole (56 mg, 0.64 mmol, 2.5 eq) was taken and dissolved in 1.5 mL of CH₃CN, and the mixture was added with NaH (22 mg, 0.64 mmol, 2.5 eq), stirred for 30 minutes at room temperature, added with a solution of SSL26-IM2 (66 mg, 0.26 mmol, 1.0 eq) dissolved in 1.0 mL of THF, and washed with 0.5 mL of THF. The reaction mixture was reacted for 3.0 hours at room temperature, and TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (32 mg, yield of 48%).

### Step 2

Compound SSL29-IM1 (32 mg, 0.12 mmol, 1.0 eq) was taken and dissolved in 1.0 mL of THF, and 0.5 mL of MeOH was added thereto. LiOH (10 mg, 0.24 mmol, 2.0 eq) was added to another EP tube, dissolved in 0.2 mL of H₂O, which was added to the reaction mixture, washed with 0.1 mL of H₂O, and reacted at room temperature for 5.0 hours. TLC (DCM:MeOH=10:1) was used to monitor the reaction until the starting material disappeared completely, and the reaction mixture was concentrated, and then the residue was purified by PTLC (CH₂Cl₂:MeOH=3:1) to obtain the product (30 mg, yield of 94%). ¹H NMR (400 MHz, Methanol-d₄) δ 8.67 (d, J=2.2 Hz, 1H), 8.04 (dd, J=8.2, 2.3 Hz, 1H), 7.52-7.41 (m, 2H), 7.29 (d, J=8.1 Hz, 1H), 6.77 (dd, J=7.9, 1.8 Hz, 1H), 6.62 (d, J=16.1 Hz, 1H), 5.27 (s, 2H). Mass: [M−H]⁻246.1.

### Example 30 Synthesis of Compound 49

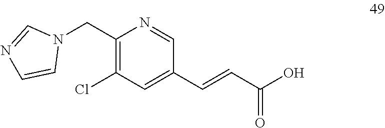 [SMILES: O=C(O)/C=C/c1cnc(Cn2ccnc2)c(Cl)c1]

### (E)-3-(6-((1H-Imidazol-1-yl)methyl)-5-chloropyridin-3-yl)acrylic acid

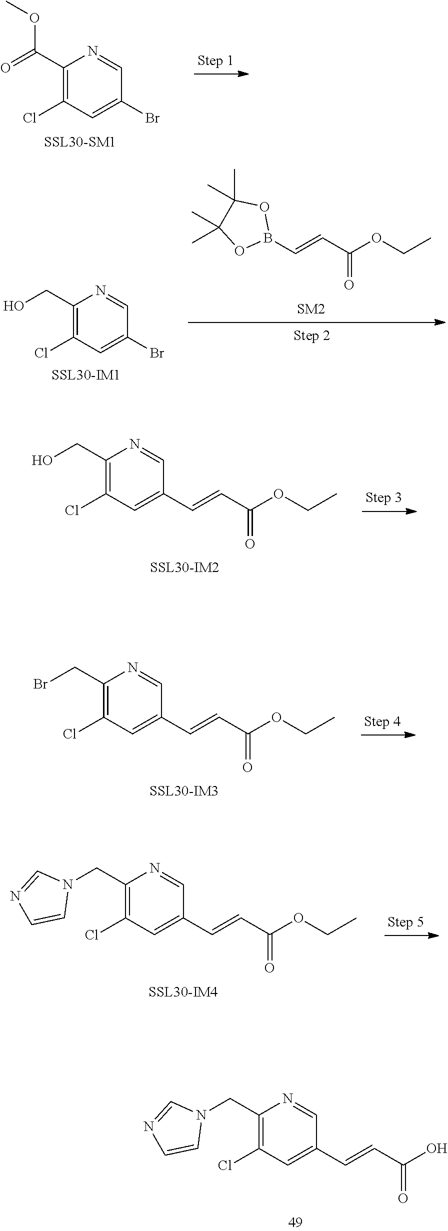

### Step 1

A 100 mL round-bottom flask was taken, and SSL30-SM1 (1.0 g, 3.99 mmol, 1.0 eq) was added thereto and dissolved with 15 mL of CH₂Cl₂, and the mixture was added with 15 mL of MeOH, placed in an ice bath for 5 minutes, and then added with NaBH₄ (302 mg, 7.98 mmol, 2.0 eq) in batches, and after the reaction was stabilized, the ice bath was removed, and the reaction mixture was reacted for 2.0 hours at room temperature. TLC (PE:EA=3:1) was used to monitor the reaction until the starting material disappeared completely. After the reaction was quenched, the reaction mixture washed with saturated NaHCO₃ solution, extracted with CH₂Cl₂, and the organic phases were combined, dried, and concentrated to obtain the white solid product (850 mg, yield of 97%).

### Step 2

SSL30-IM1 (300 mg, 1.35 mmol, 1.0 eq) was taken and dissolved with 6.0 mL of dioxane, and SM2 (457 mg, 2.02 mmol, 1.5 eq), Pd(PPh₃)₄ (16 mg, 0.01 mmol, 0.1 eq), and K₂CO₃ (559 mg, 4.05 mmol, 3.0 eq) were added thereto. Finally, 2.0 mL of H₂O was added thereto, and the reaction mixture was replaced with nitrogen three times, placed, and reacted at 100° C. for 2.0 hours. TLC (PE:EA=2:1) was used to monitor the reaction until the starting material disappeared completely. The insoluble material was removed by filtration under reduced pressure, washed with EtOAc, and the reaction mixture was concentrated and purified by column chromatography (PE:EA=4:1) to obtain the product (212 mg, yield of 65%).

### Step 3

SSL30-IM2 (212 mg, 0.88 mmol, 1.0 eq) was taken and dissolved in 9.0 mL of CH₂Cl₂, placed in an ice bath, added with CBr₄ (318 mg, 0.96 mmol, 1.1 eq) and PPh₃ (252 mg, 0.96 mmol, 1.1 eq), and the reaction mixture was reacted for 30 minutes at this temperature. TLC (PE:EA=4:1) was used to monitor the reaction until the starting material disappeared completely. The reaction mixture was concentrated and used directly in the next step.

### Step 4

The crude product of SSL30-IM3 was dissolved in 5 mL of acetonitrile, and K₂CO₃ (243 mg, 1.76 mmol, 2.0 eq) and imidazole (90 mg, 1.32 mmol, 1.5 eq) were added thereto, and then the reaction mixture was reacted for 5.0 hours at 50° C. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the solid product (145 mg, a two-step yield of 56%). ¹H NMR (400 MHz, Chloroform-d) δ 8.57 (d, J=1.9 Hz, 1H), 7.84 (d, J=1.9 Hz, 1H), 7.65 (s, 1H), 7.58 (d, J=16.1 Hz, 1H), 7.04 (d, J=10.2 Hz, 2H), 6.50 (d, J=16.1 Hz, 1H), 5.36 (s, 2H), 4.27 (q, J=7.1 Hz, 2H), 1.33 (t, J=7.1 Hz, 3H).

### Step 5

Compound SSL30-IM4 (145 mg, 0.50 mmol, 1.0 eq) was taken and dissolved in 3.0 mL of THF, and 1.5 mL of MeOH was added thereto. Another EP tube was taken, and LiOH (41 mg, 0.99 mmol, 2.0 eq) was added thereto, dissolved in 0.8 mL of H₂O, added to the reaction mixture, washed twice with 0.1 mL of H₂O, and the reaction mixture was reacted at room temperature for 5.0 hours. TLC (DCM:MeOH=10:1) was used to monitor the reaction until the starting material disappeared completely, and the reaction mixture was concentrated, and then the residue was purified by PTLC (CH₂Cl₂:MeOH=2:1) to obtain the solid product (110 mg, yield of 83%). ¹H NMR (400 MHz, Methanol-d₄) δ 8.47 (s, 1H), 7.96 (s, 1H), 7.83 (s, 1H), 7.40 (d, J=16.0 Hz, 1H), 7.12 (s, 1H), 6.99 (s, 1H), 6.63 (d, J=16.0 Hz, 1H), 5.41 (s, 2H). Mass: [M+H]⁺ 264.0.

### Example 31 Synthesis of Compound 50

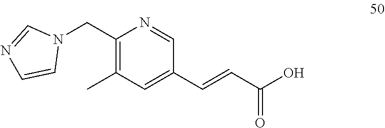 [SMILES: Cc1cc(/C=C/C(=O)O)cnc1Cn1ccnc1]

### (E)-3-(6-((1H-Imidazol-1-yl)methyl)-5-methylpyridin-3-yl)acrylic acid

### Synthetic Route

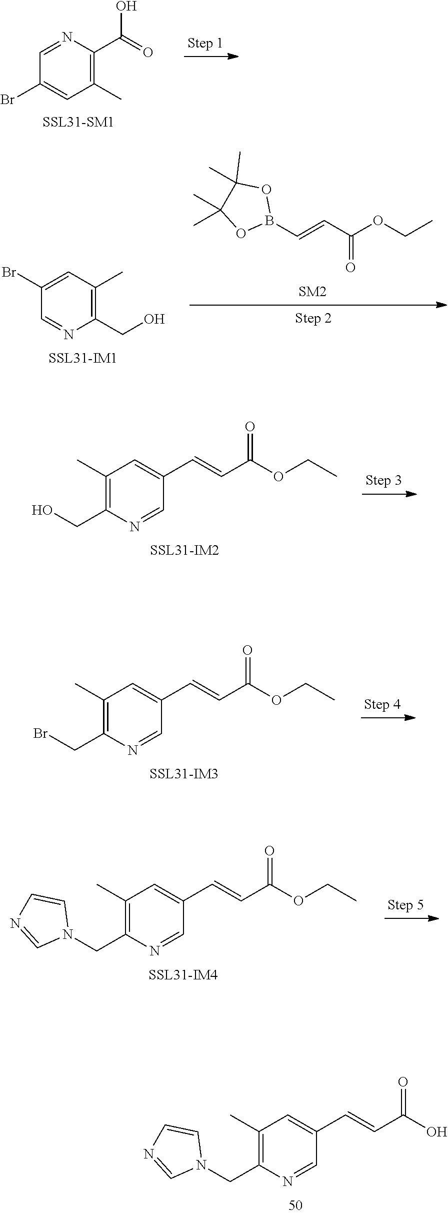

### Step 1

SSL31-SM1 was dissolved in THE in an ice bath, and the mixture was slowly added dropwise with BH₃·THF (14 mL, 13.8 mmol, 3.0 eq). After the dropwise addition was complete, the reaction mixture was heated to 70° C. and stirred for 5 hours. The complete reaction of the raw material was monitored by TLC (DCM:MeOH=20:1). The reaction was quenched with dropwise addition of methanol in an ice bath, and the solvent was removed by rotary evaporation until dryness, and then the residue was added with 40 mL of DCM and 20 mL of saturated sodium carbonate and stirred for 10 minutes. The phases were separated, and then the organic phase was subjected to rotary evaporation until dryness, and the residue was subjected to column chromatography (DCM:MeOH=50:1) to obtain the liquid product.

### Step 2

SSL31-IM1, borate SM2, Pd(dppf)Cl₂ dichloromethane complex, and potassium carbonate were added to a 100 mL single-neck flask under N₂ atmosphere, and dioxane and water were added thereto, and then the reaction mixture was heated to 100° C. and stirred for 2 hours. The complete reaction of the raw material was monitored by TLC (DCM:MeOH=30:1). After the reaction was complete, the reaction mixture was subjected to rotary evaporation until dryness, and the residue was subjected to column chromatography (DCM:MeOH=50:1) to obtain a light yellow liquid (40 mg, yield of 75%).

### Step 3

SSL31-IM2 was dissolved in 2 mL of dichloromethane, and then the mixture was added with tetrabromomethane, maintained at 0° C., added with triphenylphosphine in batches, still maintained at 0° C., and the reaction mixture was stirred for 2 hours at 0° C. After the reaction was complete, the reaction mixture was subjected to rotary evaporation until dryness, and the residue was subjected to column chromatography (PE:EA=10:1) to obtain a light yellow solid (25 mg, yield of 38%).

### Step 4

SSL31-IM3, potassium carbonate, and imidazole were dissolved in acetonitrile, then the reaction mixture was heated to 70° C. and stirred for 1 hour. The complete reaction of the raw material was monitored by TLC (DCM:CH₃OH=30:1), then the reaction mixture was subjected to rotary evaporation until dryness, and the residue was subjected to column chromatography (DCM:CH₃OH=20:1) to obtain a solid (70 mg, yield of 74%).

### Step 5

SSL31-IM4 was dissolved in tetrahydrofuran and methanol, then the mixture was added dropwise with lithium hydroxide aqueous solution in an ice bath and stirred for 2 hours. The reaction mixture was subjected to rotary evaporation until dryness, and the residue was subjected to preparative thin-layer chromatography (DCM:CH₃OH=2:1) to obtain the product (60 mg, yield of 95%). 1H NMR (400 MHz, Methanol-d4) δ 8.32-8.13 (m, 1H), 7.73-7.49 (m, 2H), 7.26 (d, J=16.0 Hz, 1H), 6.88 (dd, J=32.4, 1.3 Hz, 2H), 6.47 (d, J=16.0 Hz, 1H), 5.17 (s, 2H), 2.15 (s, 3H). Mass: [M+H]⁺ 244.1.

### Example 32 Synthesis of Compound 51

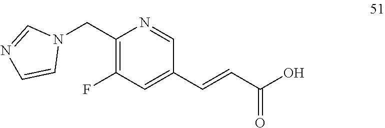 [SMILES: O=C(O)/C=C/c1cnc(Cn2ccnc2)c(F)c1]

### (E)-3-(6-((1H-Imidazol-1-yl)methyl)-5-fluoropyridin-3-yl)acrylic acid

### Synthetic Route

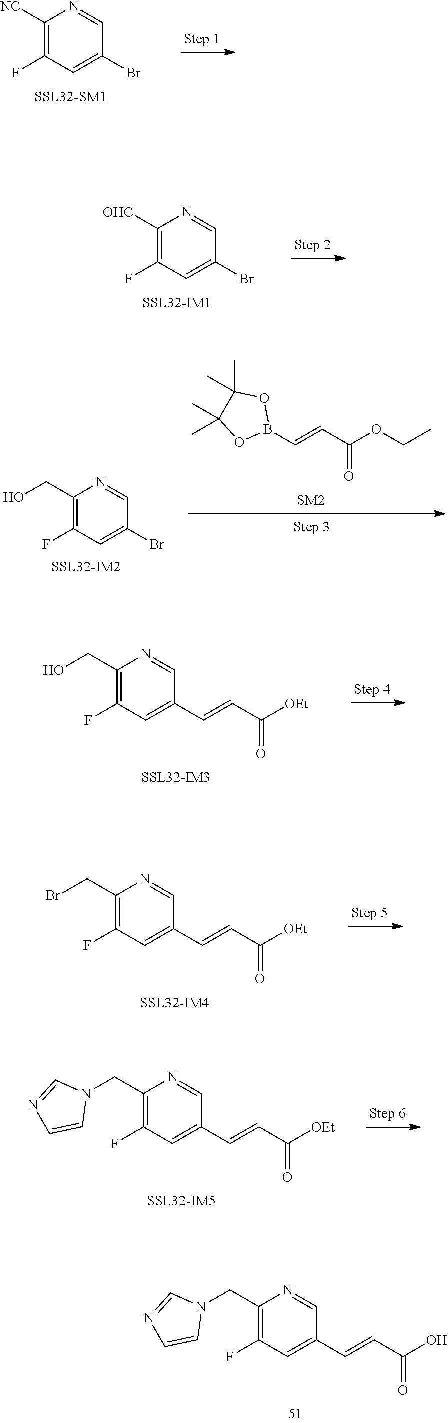

### Step 1

SSL32-SM1 was dissolved in THF at −78° C., and the mixture was slowly added dropwise with DIBAL-H (7.5 mL, 7.5 mmol, 1.5 eq). After the dropwise addition was completed, the reaction mixture was stirred for 2 hours. The complete reaction of the raw material was monitored by TLC (PE/EA 20:1). The reaction was quenched by the dropwise addition of dilute hydrochloric acid, and the reaction mixture was stirred for 30 minutes at room temperature. The solvent was removed by rotary evaporation until dryness, and the residue was added with 40 mL of DCM and 20 mL of saturated sodium chloride and stirred for 2 minutes. The phases were separated, then the organic phase was subjected to rotary evaporation until dryness, and the residue was subjected to column chromatography (PE:EA=30:1) to obtain the product.

### Step 2

SSL32-IM1 was dissolved in THE at 0° C., and the mixture was slowly added with NaBH₃ (151 mg, 3.9 mmol, 3.0 eq). After the dropwise addition was completed, the reaction mixture was stirred for 2 hours. The complete reaction of the raw material was monitored by TLC (DCM/MeOH 100:1). The pH was adjusted to 7 by the dropwise addition of dilute hydrochloric acid, and the mixture was stirred for 10 minutes at room temperature. The mixture was filtered, and the solvent was removed by rotary evaporation until dryness, and then the residue was added with 40 mL of DCM and 20 mL of saturated sodium chloride and stirred for 2 minutes. The phases were separated, then the organic phase was subjected to rotary evaporation until dryness, and the residue was subjected to column chromatography (DCM/MeOH 50:1) to obtain the product.

### Step 3

SSL32-IM2 (50 mg, 0.24 mmol, 1.0 eq), borate SM2 (84 mg, 0.37 mmol, 1.5 eq), Pd(dppf)Cl₂ dichloromethane complex (20 mg, 0.024 mmol, 0.1 eq), and potassium carbonate (100 mg, 0.72 mmol, 3.0 eq) were added to a 100 mL single-neck flask under N₂ atmosphere, and dioxane and water was added thereto, and then the reaction mixture was heated to 100° C. and stirred for 2 hours. TLC (DCM/MeOH 30:1) was used to monitor the reaction until the reaction was complete. After the reaction was complete, the reaction mixture was subjected to rotary evaporation until dryness, and the residue was subjected to column chromatography (DCM/MeOH 50:1) to obtain a light yellow liquid (40 mg, yield of 75%).

### Step 4

SSL32-IM3 (50 mg, 0.23 mmol, 1.0 eq) was dissolved in dichloromethane, and tetrabromomethane (90 mg, 0.27 mmol, 1.2 eq) was added thereto, maintained at 0° C., and triphenylphosphine (71 mg, 0.27 mmol, 1.2 eq) was added thereto in batches, still maintained at 0° C., and then the reaction mixture was stirred for 2 hours at 0° C. After the reaction was complete, the reaction mixture was subjected to rotary evaporation until dryness, and the residue was subjected to column chromatography (PE/EA 10:1) to obtain a light yellow solid (25 mg, yield of 38%).

### Step 5

SSL32-IM4 (100 mg, 0.35 mmol, 1.0 eq), potassium carbonate (100 mg, 0.70 mmol, 2.0 eq), and imidazole (50 mg, 0.70 mmol, 2.0 eq) were dissolved in acetonitrile, and then the reaction mixture was heated to 70° C. and stirred for 1 hour. The complete reaction of the raw material was monitored by TLC (DCM:CH₃OH=30:1), then the reaction mixture was subjected to rotary evaporation until dryness, and the residue was subjected to column chromatography (DCM/CH₃OH 20:1) to obtain a solid (70 mg, yield of 74%).

### Step 6

SSL32-IM5 (70 mg, 0.26 mmol, 1.0 eq) was dissolved in tetrahydrofuran and methanol, then the mixture was added dropwise with lithium hydroxide aqueous solution (22 mg, 0.52 mmol, 2.0 eq) in an ice bath and stirred for 2 hours. The reaction mixture was subjected to rotary evaporation until dryness, and the residue was subjected to preparative thin-layer chromatography (DCM/CH₃OH 2:1) to obtain the product (60 mg, yield of 95%). ¹H NMR (400 MHz, Methanol-d₄) δ 8.45 (s, 1H), 7.87-7.69 (m, 2H), 7.38-7.24 (m, 1H), 7.11 (d, J=1.6 Hz, 1H), 6.92 (d, J=1.2 Hz, 1H), 6.60 (d, J=16.0 Hz, 1H), 5.20-5.14 (m, 2H).

Mass: [M+H]⁺ 248.1.

### Example 33 Synthesis of Compound 52 and Compound 53

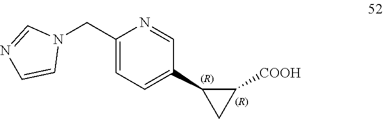

### (1R,2R)-2-(6-((1H-Imidazol-1-yl)methyl)pyridin-3-yl)cyclopropane-1-carboxylic acid

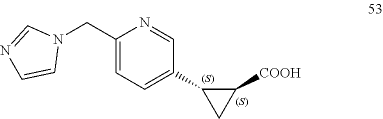

### (1S,2S)-2-(6-((1H-Imidazol-1-yl)methyl)pyridin-3-yl)cyclopropane-1-carboxylic acid

### Synthetic Route

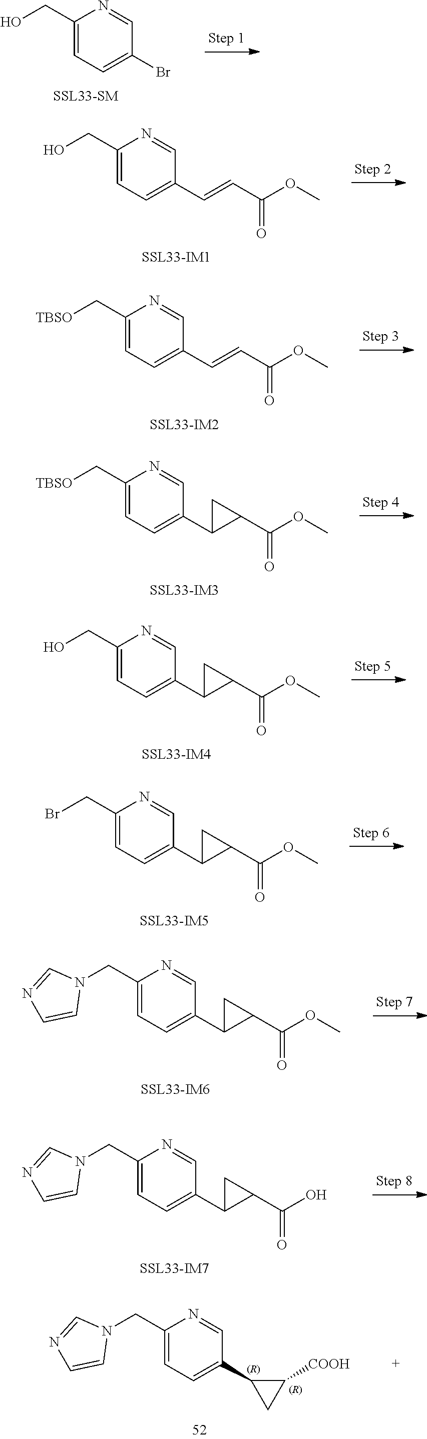

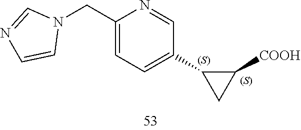

### Step 1

A 50 mL round-bottom flask was taken, and SSL33-SM (2.0 g, 10.76 mmol, 1.0 eq), palladium acetate (242 mg, 1.08 mmol, 0.1 eq), POT (658 mg, 2.16 mmol, 0.2 eq), triethylamine (4.50 mL, 32.32 mmol, 3.0 eq), and methyl acrylate (9.26 g, 107.5 mmol, 10 eq) were added thereto and dissolved in 20 mL of DMF, and the air in the system was replaced three times with N₂ after the dissolution was complete. The reaction mixture was reacted for 5.0 hours at 80° C., and TLC (PE:EA=1:1) was used to monitor the reaction until the starting material disappeared completely, and then the reaction was terminated. The reaction mixture was washed with water and extracted 5 times with ethyl acetate until no product remained in the aqueous phase. The organic phases were combined, concentrated to remove the solvent, and the residue was used directly in the next step.

### Step 2

SSL33-IM1 (180 mg, 0.93 mmol, 1.0 eq) was taken and dissolved in 15.0 mL of CH₂Cl₂, and imidazole (1.5 g, 21.5 mmol, 2.0 eq) and TBSCl (2.1 g, 13.98 mmol, 1.3 eq) were added thereto, and then the reaction mixture was reacted at room temperature for 5.0 hours. TLC (CH₂Cl₂:MeOH=10:1) was used to monitor the reaction until the starting material disappeared completely. The reaction mixture was washed with water and extracted with DCM, and the organic phases were combined, dried, concentrated, and then purified by column chromatography (PE:EA=10:1) to obtain the solid product (1.3 g, a two-step yield of 40%).

### Step 3

9.0 mL of DMSO and NaH (176 mg, 4.40 mmol, 1.3 eq) were added to a 50 mL reaction flask, and 5 minutes after the addition, trimethylsulfoxonium iodide (968 mg, 4.40 mmol, 1.3 eq) was added thereto, and the reaction mixture was stirred at room temperature for 1.0 hour to obtain a clear and transparent solution. SSL33-IM2 (1.04 g, 3.38 mmol, 1.0 eq) was added to another 25 mL reaction flask, dissolved in 3.0 mL of THF, which was added to the reaction mixture and washed twice with 1.0 mL of THF. The reaction mixture was reacted for 2.0 hours at room temperature, and TLC (PE/EA=5:1) was used to monitor the reaction until the starting material disappeared completely. The reaction mixture was concentrated and purified by column chromatography (PE:EA=5:1), after which the product components were combined, concentrated, and purified again by PTLC to obtain the product (30 mg, yield of 3%).

### Step 4

SSL33-IM3 (30 mg, 0.10 mmol, 1.0 eq) was added to a 50 mL reaction flask, dissolved in 1.0 mL of THF, and 1.0 M TBAF (0.20 mL, 0.20 mmol, 2.0 eq) was added thereto, and then the reaction mixture was reacted at room temperature for 1.0 hour. TLC (PE:EA=5:1) was used to monitor the reaction until the starting material disappeared completely. The reaction mixture was concentrated, and the residue was purified by PTLC (CH₂Cl₂:MeOH=10:1) to obtain the product (20 mg, yield of 95%).

### Step 5

SSL33-IM4 (20 mg, 0.10 mmol, 1.0 eq) was taken and dissolved in 1.0 mL of CH₂Cl₂, placed in an ice bath, and CBr₄ (42 mg, 0.13 mmol, 1.3 eq) and PPh₃ (34 mg, 0.13 mmol, 1.3 eq) were added thereto, and then the reaction mixture was reacted for 20 minutes at this temperature. TLC (CH₂Cl₂:MeOH=10:1) was used to monitor the reaction until the starting material disappeared completely. The reaction mixture was concentrated and used directly in the next step.

### Step 6

The crude product of SSL33-IM5 was taken and dissolved with 1.0 mL of CH₃CN, and then imidazole (14 mg, 0.20 mmol, 2.0 eq) and K₂CO₃ (42 mg, 0.30 mmol, 3.0 eq) were added thereto, and the reaction mixture was reacted at 40° C. for 4.0 hours. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (18 mg, a two-step yield of 73%).

### Step 7

Compound SSL33-IM6 (18 mg, 0.07 mmol, 1.0 eq) was taken and dissolved in 0.5 mL of THF, and 0.3 mL of MeOH was added thereto. LiOH (6.0 mg, 0.14 mmol, 2.0 eq) was added to another EP tube, dissolved in 0.3 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=1:1) to obtain the product (12 mg, yield of 83%). ¹H NMR (400 MHz, Methanol-d₄) δ 8.37 (d, J=2.2 Hz, 1H), 7.81 (d, J=1.2 Hz, 1H), 7.48 (dd, J=8.1, 2.3 Hz, 1H), 7.17-7.12 (m, 2H), 7.01 (d, J=1.5 Hz, 1H), 5.28 (s, 2H), 2.40 (ddd, J=9.5, 6.1, 4.1 Hz, 1H), 1.80 (ddd, J=8.5, 5.5, 4.2 Hz, 1H), 1.52 (ddd, J=9.4, 5.4, 4.3 Hz, 1H), 1.21 (ddd, J=8.4, 6.1, 4.3 Hz, 1H). Mass: [M+H]⁺ 244.1.

### Step 8

SSL33-IM7 was obtained and purified by a chiral preparative column to obtain compound 52 and Compound 53.

### Example 34 Synthesis of Compound 54 and Compound 55

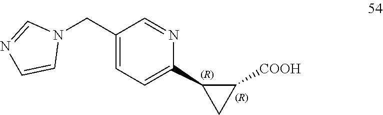

### (1R,2R)-2-(5-((1H-Imidazol-1-yl)methyl)pyridin-2-yl)cyclopropane-1-carboxylic acid

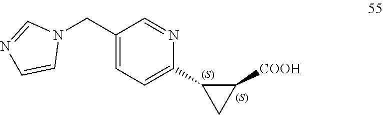

### (1S,2S)-2-(5-((1H-Imidazol-1-yl)methyl)pyridin-2-yl)cyclopropane-1-carboxylic acid

### Synthetic Route

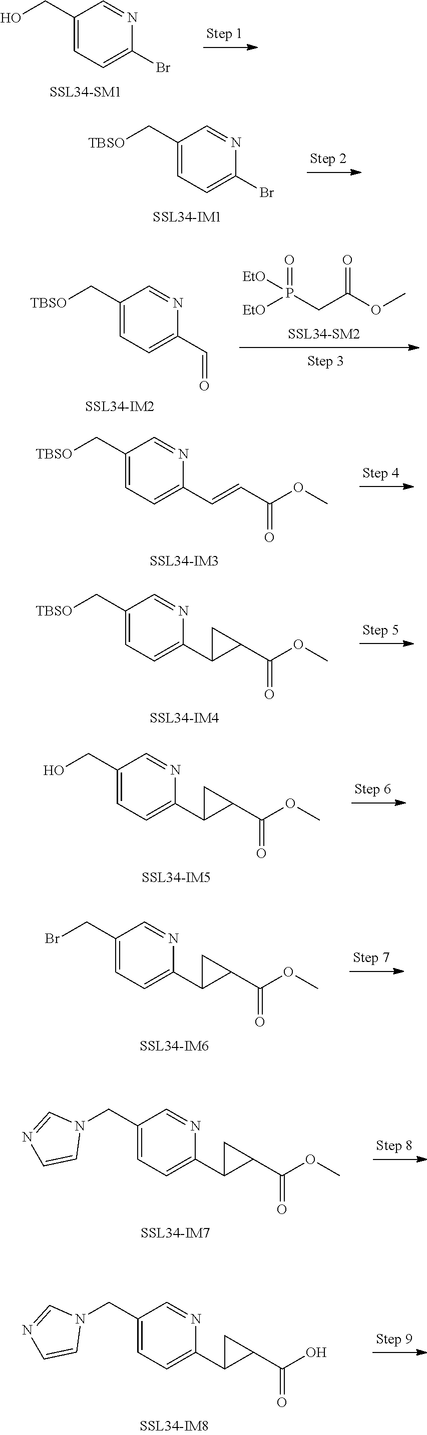

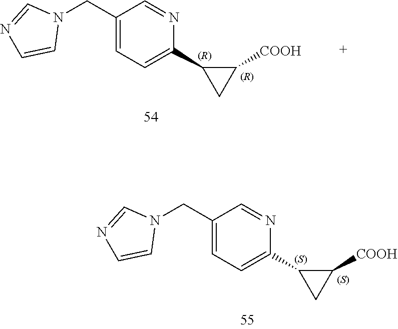

### Step 1

A 100 mL round-bottom flask was taken, and SSL34-SM1 (5.0 g, 26.74 mmol, 1.0 eq) and imidazole (3.6 g, 53.48 mmol, 2.0 eq) were added thereto and dissolved in 20 mL of DCM, and then TBSCl (5.2 g, 37.77 mmol, 1.3 eq) was added thereto. The reaction mixture was reacted at room temperature for 4.0 hours. TLC (PE:EA=1:1) was used to monitor the reaction until the starting material disappeared completely. The reaction mixture was washed with water and extracted with DCM, and the organic phases were combined, concentrated, and then purified by column chromatography to obtain the product (8.0 g, yield of 98%).

### Step 2

SSL34-IM1 (5.0 g, 16.54 mmol, 1.0 eq) was taken, dissolved in 12 mL of THF, and placed at −78° C. The mixture was slowly added with 2.4 M n-BuLi (7.24 mL, 17.37 mmol, 1.05 eq) after 10 minutes and reacted for 30 minutes at −78° C., and then the reaction mixture was slowly added with DMF (1.4 mL, 18.19 mmol, 1.1 eq) and reacted for another 2 hours. TLC (PE:EA=3:1) was used to monitor the reaction until the starting material disappeared completely. The reaction was quenched with saturated ammonium chloride solution, and after the reaction mixture was warmed to room temperature, the organic phase was washed with water and extracted with DCM. The organic phases were combined, concentrated, and used directly in the next step.

### Step 3

A 100 mL reaction flask was taken, SSL34-SM2 (4.6 mL, 24.81 mmol, 1.5 eq) was added thereto, dissolved with 15 mL of THF, placed in an ice bath, and slowly added with NaH (990 mg, 24.81 mmol, 1.5 eq) in batches. After the reaction was stabilized, the ice bath was removed, and the reaction mixture was reacted for 30 minutes at room temperature. Another 25 mL reaction flask was taken, and unpurified SSL34-IM2 was added thereto, dissolved in 3 mL of THF, and then added to the reaction mixture and washed twice with 1 mL of THF. The reaction mixture was reacted for 4.0 hours at room temperature, and TLC (PE:EA=3:1) was used to monitor the reaction until the starting material disappeared completely. The reaction was quenched with saturated ammonium chloride solution, and after the reaction mixture was warmed to room temperature, the organic phase washed with water, and extracted with DCM. The organic phases were combined, concentrated, and purified by column chromatography to obtain the product (3.3 g, a two-step yield of 65%).

### Step 4

8.0 mL of DMSO and NaH (169 mg, 4.23 mmol, 1.3 eq) were added to a 50 mL reaction flask, and 5 minutes after the addition, trimethylsulfoxonium iodide (930 mg, 4.23 mmol, 1.3 eq) was added thereto, and the reaction mixture was stirred at room temperature for 1.0 hour to obtain a clear and transparent solution. SSL34-IM3 (66 mg, 0.26 mmol, 1.0 eq) was added to another 25 mL reaction flask, dissolved in 3.0 mL of THF, which was added to the reaction mixture and washed twice with 1.0 mL of THF. The reaction mixture was reacted for 2.0 hours at room temperature, and TLC (PE:EA=5:1) was used to monitor the reaction until the starting material disappeared completely. The reaction mixture was concentrated and subjected to column chromatography (PE:EA=5:1), after which the product components were combined to obtain the crude product (200 mg), which was used in the next step.

### Step 5

The crude product of SSL34-IM4 (200 mg, 0.62 mmol, 1.0 eq) was added to a 50 mL reaction flask, dissolved in 4.0 mL of THF, and 1.0 M TBAF (0.20 mL, 0.20 mmol, 2.0 eq) was added thereto, and then the reaction mixture was reacted at room temperature for 2.0 hours. TLC (PE:EA=5:1) was used to monitor the reaction until the starting material disappeared completely. The reaction mixture was concentrated and purified by PTLC (PE:EA=1:1) to obtain the crude product (120 mg, a two-step yield of 17%).

### Step 6

SSL34-IM5 (120 mg, 0.58 mmol, 1.0 eq) was taken and dissolved in 5.0 mL of CH₂Cl₂, placed in an ice bath, added with CBr₄ (250 mg, 0.75 mmol, 1.3 eq) and PPh₃ (197 mg, 0.75 mmol, 1.3 eq), and the reaction mixture was reacted for 30 minutes at this temperature. TLC (CH₂Cl₂:MeOH=10:1) was used to monitor the reaction until the starting material disappeared completely. The reaction mixture was concentrated and used directly in the next step.

### Step 7

The crude product of SSL34-IM6 was taken and dissolved with 5.0 mL of CH₃CN, and then imidazole (79 mg, 1.16 mmol, 2.0 eq) and K₂CO₃ (240 mg, 1.74 mmol, 3.0 eq) were added thereto, and the reaction mixture was reacted at room temperature for 5.0 hours. TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the crude product, which was then purified by HPLC to obtain the product (33 mg, a two-step yield of 22%).

### Step 8

Compound SSL34-IM7 (33 mg, 0.13 mmol, 1.0 eq) was taken and dissolved in 1.0 mL of THF, and 0.5 mL of MeOH was added thereto. LiOH (11 mg, 0.14 mmol, 2.0 eq) was added to another EP tube, dissolved in 0.4 mL of H₂O, which was added to the reaction mixture and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=2:1) to obtain the product (20 mg, yield of 63%). ¹H NMR (400 MHz, Methanol-d₄) δ 8.34 (d, J=2.2 Hz, 1H), 7.85 (s, 1H), 7.57 (dd, J=8.1, 2.4 Hz, 1H), 7.29 (dd, J=8.1, 0.8 Hz, 1H), 7.16 (d, J=1.3 Hz, 1H), 7.03 (d, J=1.3 Hz, 1H), 5.25 (s, 2H), 2.55 (ddd, J=8.8, 6.0, 3.9 Hz, 1H), 2.05 (ddd, J=9.1, 5.5, 3.9 Hz, 1H), 1.55-1.43 (m, 2H). Mass: [M+H]⁺ 244.1.

### Step 9

SSL34-IM8 was obtained and purified by a chiral preparative column to obtain compound 54 and Compound 55.

### Example 35 Synthesis of Compound 56

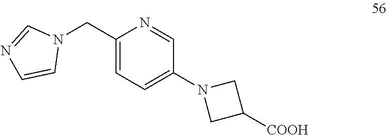 [SMILES: O=C(O)C1CN(c2ccc(Cn3ccnc3)nc2)C1]

### 1-(6-((1H-Imidazol-1-yl)methyl)pyridin-3-yl)azetidine-3-carboxylic acid

### Synthetic Route

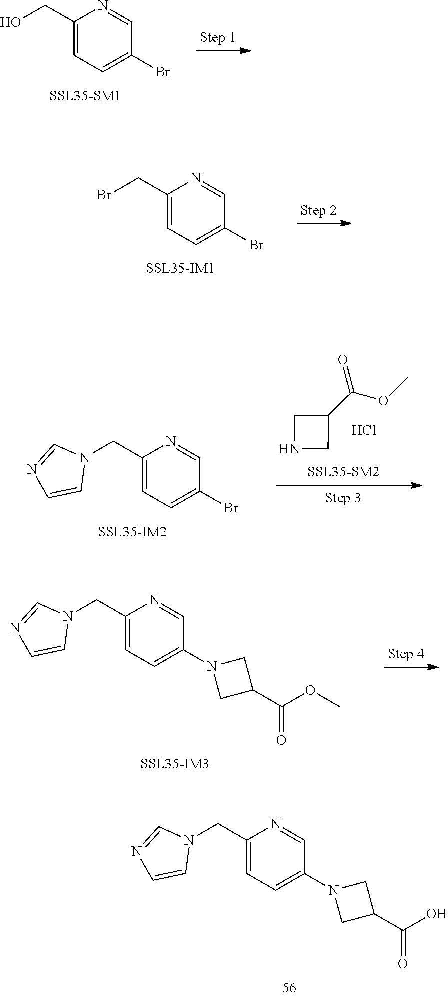

### Step 1

A 100 mL round-bottom flask was taken, and SSL35-SM1 (0.5 g, 2.67 mmol, 1.0 eq) was added thereto, dissolved in 20 mL of THF, and then PPh₃ (1.4 g, 5.34 mmol, 2.0 eq) and CBr₄ (1.3 g, 4.0 mmol, 1.5 eq) were sequentially added thereto, and the reaction mixture was reacted for 1.0 hour at room temperature. TLC (PE:EA=1:1) was used to monitor the reaction, and the reaction was terminated when the starting material disappeared completely.

The solvent was concentrated and the residue was used directly in the next step.

### Step 2

The crude product of SSL35-IM1 was taken and dissolved in 15 mL of acetonitrile, and K₂CO₃ (1.5 g, 10.68 mmol, 4.0 eq) and imidazole (545 mg, 8.01 mmol, 3.0 eq) were added thereto, and then the reaction mixture was reacted for 3.0 hours at room temperature. TLC (CH₂Cl₂:MeOH=10:1) was used to monitor the reaction until the starting material disappeared completely. The reaction mixture was concentrated, and the residue was purified by column chromatography (CH₂Cl₂:MeOH=20:1) to obtain the solid product (450 mg, a two-step yield of 71%).

### Step 3

Compound SSL35-IM2 (240 mg, 1.01 mmol, 1.0 eq) was taken and dissolved in 6.0 mL of t-BuOH, and then SM2 (300 mg, 2.02 mmol, 2.0 eq), Pd(OAc)₂ (23 mg, 0.10 mmol, 0.1 eq), RuPhos (94 mg, 0.20 mmol, 0.2 eq), and Cs₂CO₃ (990 mg, 3.03 mmol, 3.0 eq) were sequentially added thereto. The reaction mixture was replaced three times with nitrogen and reacted for 6.0 hours at 95° C. TLC (CH₂Cl₂:MeOH=10:1) was used to monitor the reaction until the starting material disappeared completely. The reaction mixture was concentrated and filtered under reduced pressure, and washed with dichloromethane, and the filtrate was concentrated and purified by PTLC (CH₂Cl₂:MeOH=10:1) to obtain the solid product (103 mg, yield of 37%). ¹H NMR (300 MHz, Chloroform-d) δ 7.80 (d, J=2.9 Hz, 1H), 7.58 (s, 1H), 7.07 (s, 1H), 6.96 (s, 1H), 6.87 (d, J=8.6 Hz, 1H), 6.68 (dd, J=8.4, 2.8 Hz, 1H), 5.12 (s, 2H), 4.17-4.04 (m, 4H), 3.75 (s, 3H), 3.67-3.57 (m, 1H).

### Step 4

Compound SSL35-IM3 (110 mg, 0.38 mmol, 1.0 eq) was taken and dissolved in 3.0 mL of THF, and 1.0 mL of MeOH was added thereto. Another 2.0 mL reaction flask was taken, and LiOH (31 mg, 0.76 mmol, 2.0 eq) was added thereto, dissolved in 0.7 mL of H₂O, added to the reaction mixture, washed twice with 0.15 mL of H₂O, and the reaction mixture was reacted at room temperature for 2.0 hours. TLC (DCM:MeOH=10:1) was used to monitor the reaction until the starting material disappeared completely, and the reaction mixture was concentrated, and then the residue was dissolved with methanol and filtered, and the filtrate was concentrated to obtain the white solid product (90 mg, yield of 92%). ¹H NMR (400 MHz, Methanol-d₄) δ 7.84-7.68 (m, 2H), 7.14 (d, J=8.6 Hz, 1H), 7.10 (s, 1H), 6.95 (s, 1H), 6.88 (d, J=8.7 Hz, 1H), 5.16 (s, 2H), 4.09 (t, J=7.8 Hz, 2H), 4.00 (t, J=6.9 Hz, 2H), 3.45 (d, J=7.7 Hz, 1H). Mass: [M+H]⁺ 259.0.

### Example 36 Synthesis of Compound 57

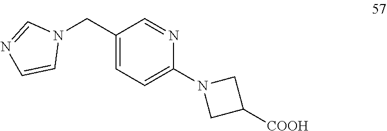 [SMILES: O=C(O)C1CN(c2ccc(Cn3ccnc3)cn2)C1]

### 1-(5-((1H-Imidazol-1-yl)methyl)pyridin-2-yl)azetidine-3-carboxylic acid

### Synthetic Route

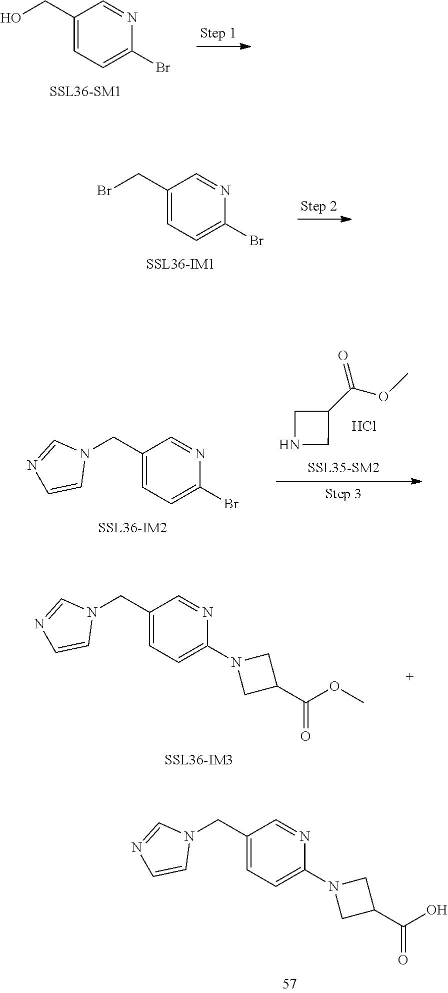

### Step 1

A 100 mL round-bottom flask was taken, and SSL36-SM1 (0.5 g, 2.67 mmol, 1.0 eq) was added thereto, dissolved in 20 mL of THF, and then PPh₃ (1.4 g, 5.34 mmol, 2.0 eq) and CBr₄ (1.3 g, 4.0 mmol, 1.5 eq) were sequentially added thereto, and the reaction mixture was reacted for 1.0 hour at room temperature. TLC (PE:EA=1:1) was used to monitor the reaction, and the reaction was terminated when the starting material disappeared completely. The solvent was concentrated and the residue was used directly in the next step.

### Step 2

The crude product of SSL36-IM1 was taken and dissolved in 10 mL of acetonitrile, and K₂CO₃ (750 mg, 5.34 mmol, 2.0 eq) and imidazole (273 mg, 401 mmol, 1.5 eq) were added thereto, and then the reaction mixture was reacted for 3.0 hours at room temperature. TLC (CH₂Cl₂:MeOH=10:1) was used to monitor the reaction until the starting material disappeared completely. The reaction mixture was concentrated, and the residue was purified by column chromatography (CH₂Cl₂:MeOH=20:1) to obtain the solid product (320 mg, a two-step yield of 50%).

### Step 3

Compound SSL36-IM2 (170 mg, 0.71 mmol, 1.0 eq) was taken and dissolved in 6.0 mL of t-BuOH, and then SSL35-SM2 (217 mg, 1.43 mmol, 2.0 eq), Pd(OAc)₂ (16 mg, 0.07 mmol, 0.1 eq), RuPhos (63 mg, 0.14 mmol, 0.2 eq), and Cs₂CO₃ (694 mg, 2.14 mmol, 3.0 eq) were sequentially added thereto. The reaction mixture was replaced three times with nitrogen and reacted for 6.0 hours at 100° C. TLC (CH₂Cl₂:MeOH=10:1) was used to monitor the reaction until the starting material disappeared completely. The reaction mixture was concentrated and filtered under reduced pressure, washed with dichloromethane, and the filtrate was concentrated and purified by PTLC (CH₂Cl₂:MeOH=2:1) to obtain the small polar product of SSL36-IM3 (86 mg, yield of 44%) and the large polar product 57 (38 mg, yield of 21%). SSL36-IM3: ¹H NMR (300 MHz, Chloroform-d) δ 8.08 (d, J=2.4 Hz, 1H), 7.53 (s, 1H), 7.28 (d, J=2.5 Hz, 1H), 7.07 (s, 1H), 6.87 (s, 1H), 6.28 (d, J=8.5 Hz, 1H), 4.98 (s, 2H), 4.25-4.18 (m, 4H), 3.76 (s, 3H), 3.63-3.55 (m, 1H). 57: ¹H NMR (400 MHz, Methanol-d₄) δ 7.99 (d, J=2.3 Hz, 1H), 7.75 (s, 1H), 7.47 (dd, J=8.7, 2.4 Hz, 1H), 7.10 (t, J=1.4 Hz, 1H), 6.97 (s, 1H), 6.40 (d, J=8.6 Hz, 1H), 5.09 (s, 2H), 4.18-4.11 (m, 4H), 3.46-3.38 (m, 1H). Mass: [M−H]⁻257.1.

### Example 37 Synthesis of Compound 58

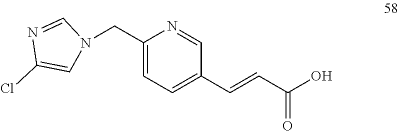 [SMILES: O=C(O)/C=C/c1ccc(Cn2cnc(Cl)c2)nc1]

### (E)-3-(6-((4-Chloro-1H-imidazol-1-yl)methyl)pyridin-3-yl)acrylic acid

### Synthetic Route

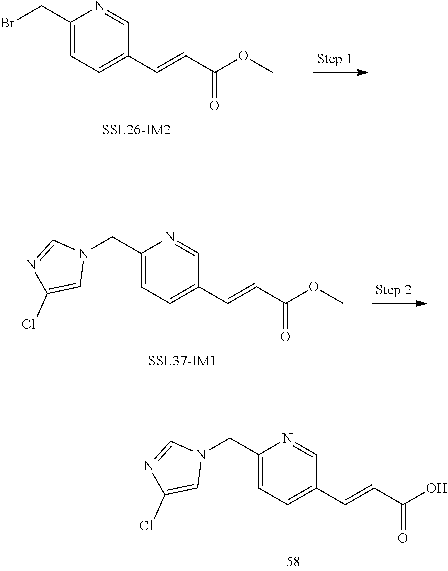 [SMILES: COC(=O)/C=C/c1ccc(CBr)nc1 ; COC(=O)/C=C/c1ccc(Cn2cnc(Cl)c2)nc1 ; O=C(O)/C=C/c1ccc(Cn2cnc(Cl)c2)nc1]

### Step 1

5-Chloroimidazole (120 mg, 1.17 mmol, 2.0 eq) was taken and dissolved in 4.0 mL of CH₃CN, and the mixture was added with NaH (47 mg, 1.17 mmol, 2.0 eq), stirred for 30 minutes at room temperature, added with a solution of SSL26-IM2 (150 mg, 0.59 mmol, 1.0 eq) dissolved in 1.0 mL of THF, and washed with 0.5 mL of THF. The reaction mixture was reacted for 3.0 hours at room temperature, and TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (40 mg, yield of 25%).

### Step 2

Compound SSL37-IM1 (40 mg, 0.14 mmol, 1.0 eq) was taken and dissolved in 1.0 mL of THF, and 0.5 mL of MeOH was added thereto. LiOH (12 mg, 0.76 mmol, 2.0 eq) was added to another EP tube, dissolved in 0.2 mL of H₂O, which was added to the reaction mixture, washed with 0.1 mL of H₂O, and reacted at room temperature for 5.0 hours. TLC (DCM:MeOH=10:1) was used to monitor the reaction until the starting material disappeared completely, and the reaction mixture was concentrated, and then the residue was purified by PTLC (CH₂Cl₂:MeOH=3:1) to obtain the product (24 mg, yield of 67%). ¹H NMR (400 MHz, Methanol-d₄) δ 8.67 (d, J=2.1 Hz, 1H), 8.03 (dd, J=8.1, 2.3 Hz, 1H), 7.72 (d, J=1.6 Hz, 1H), 7.48 (d, J=16.0 Hz, 1H), 7.30 (d, J=8.0 Hz, 1H), 7.16 (d, J=1.6 Hz, 1H), 6.63 (d, J=16.0 Hz, 1H), 5.31 (s, 2H). Mass: [M+H]⁺ 264.0.

### Example 38 Synthesis of Compound 59

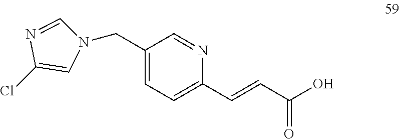 [SMILES: O=C(O)/C=C/c1ccc(Cn2cnc(Cl)c2)cn1]

### (E)-3-(5-((4-Chloro-1H-imidazol-1-yl)methyl)pyridin-2-yl)acrylic acid

### Synthetic Route

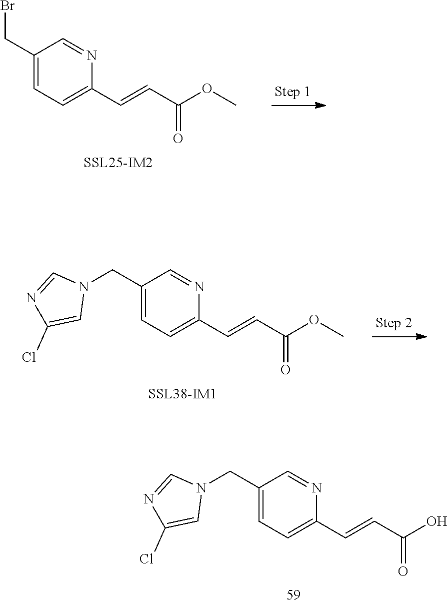 [SMILES: COC(=O)/C=C/c1ccc(CBr)cn1 ; COC(=O)/C=C/c1ccc(Cn2cnc(Cl)c2)cn1 ; O=C(O)/C=C/c1ccc(Cn2cnc(Cl)c2)cn1]

### Step 1

5-Chloroimidazole (213 mg, 2.08 mmol, 2.0 eq) was taken and dissolved in 6 mL of CH₃CN, and the mixture was added with NaH (83 mg, 2.08 mmol, 2.0 eq), stirred for 30 minutes at room temperature, added with the crude product of SSL25-IM2 dissolved in 2.0 mL of CH₃CN, and washed twice with 1.0 mL of CH₃CN. The reaction mixture was reacted for 4.0 hours at room temperature, and TLC (CH₂Cl₂:MeOH=10:1) was used to monitor the reaction until the starting material disappeared completely. The reaction mixture was concentrated, and the residue was purified by PTLC (CH₂Cl₂:MeOH=10:1) to obtain the product (94 mg, a two-step yield of 33%).

### Step 2

Compound SSL38-IM1 (94 mg, 0.34 mmol, 1.0 eq) was taken and dissolved in 2.0 mL of THF, and 1.0 mL of MeOH was added thereto. LiOH (28 mg, 0.68 mmol, 2.0 eq) was added to another EP tube, dissolved in 0.5 mL of H₂O, which was added to the reaction mixture, washed with 0.2 mL of H₂O, and reacted at room temperature for 4.0 hours. TLC (DCM:MeOH=10:1) was used to monitor the reaction until the starting material disappeared completely, and the reaction mixture was concentrated, and then the residue was purified by PTLC (CH₂Cl₂:MeOH=3:1) to obtain the product (86 mg, yield of 96%). ¹H NMR (400 MHz, Methanol-d₄) δ 8.52 (d, J=2.2 Hz, 1H), 7.75 (d, J=1.6 Hz, 1H), 7.71 (dd, J=8.1, 2.3 Hz, 1H), 7.62 (d, J=8.1 Hz, 1H), 7.50 (d, J=15.8 Hz, 1H), 7.17 (d, J=1.6 Hz, 1H), 6.87 (d, J=15.9 Hz, 1H), 5.28 (s, 2H). Mass: [M−H]⁻262.0.

Effect Example 1: Analysis of the result of the experimental study of the inhibitory effect of the test compounds on platelet aggregation

### 1. Experimental Method 1

### 1.1 Grouping

Several experimental groups were established, including a blank control group, an AA (arachidonic acid) model control group, and various test compounds. Each test compound had three concentration groups: 10⁻³ M, 10⁻⁴ M, and 10⁻⁵ M. The data from the blank and model control group were shared among the different drug concentration groups as a control.

### 1.2 Preparation of Rat Test Plasma

Male SD rats were anesthetized with 3% chloral hydrate (300 mg/kg) through intraperitoneal injection. After being fixed in a supine position, the skin of the neck was cut about 3 cm, bluntly separated with a hemostat, and the trachea was exposed after the common carotid artery was separated. The distal end of the common carotid artery was ligated with a fine thread, and the proximal end was clamped with an arterial clamp for vascular intubation. After the intubation was fixed, the artery clamp was opened, and the blood was placed into a blood collection tube containing 3.8% trisodium citrate so that the blood was mixed well with the 3.8% trisodium citrate in a volume ratio of 9:1.

The prepared anticoagulant blood was mixed well and centrifuged for 10 minutes at 500 rpm, then the upper layer of plasma was aspirated to obtain platelet-rich plasma (PRP), and the PRP was centrifuged again for 5 minutes at 3,000 rpm. The supernatant was discarded, and the platelet precipitate was obtained, which was washed once with Ca²⁺-free HEPES-Tyrode's buffer, then centrifuged at 3,000 rpm for 5 minutes. The supernatant was discarded, and the platelets were resuspended with Ca²⁺-free HEPES-Tyrode's buffer to obtain the washed platelets.

### 1.3 Inhibitory Effect of the Test Compound on AA-Induced Platelet Aggregation in Rats In Vitro

First, 300 μL of Ca²⁺-containing HEPES-Tyrode's solution was taken into the test cup, put into the test wells, and calibrated by pressing the “PPP” key. Subsequently, 240 μL of the washed platelets were added to the test cup, then added with 30 μL of different concentrations of drug solution, incubated for 10 minutes at 37° C. in a preheated bath, and then put into the test wells. 3 μL of 100× CaCl₂ solution (final concentration of 0.2 g/L) and 30 μL of inducer (final concentration of AA of 800 μM) were added to the test wells, and then the “Start” key was pressed immediately. The maximum aggregation rate was recorded every 60 s. The measurement was repeated three times for each concentration for 10 minutes, and the aggregation curve was plotted.

The platelet aggregation inhibition rate was calculated with the following formula:

Inhibition rate (%)=(maximum aggregation rate in the model control group−maximum aggregation rate in the administration group)/maximum aggregation rate in the model control group×100%

### 1.4 Statistical Method

All data were expressed as mean±standard deviation (mean±SD). SPSS 22.0 software was used for statistical analysis of the data. Intergroup data were analyzed using one-way ANOVA, with P<0.05 considered as statistically significant and P<0.01 considered as highly significant.

### 1.5 Experimental Results

TABLE 1
Platelet aggregation rate and inhibition rate of each group
of drugs at 4 minutes (mean ± SD, n = 3)

| Group | Aggregation rate | Inhibition rate (%) |
|---|---|---|
| Blank control group | 1.9 ± 0.4 | — |
| AA model group | 41.2 ± 4.2** | — |
| Ozagrel (1 * 10⁻³M) | 13.2 ± 1.8^{##} | 68.0 |
| 1 (1 * 10⁻³M) | 20.8 ± 1.4^{##} | 49.5 |
| 2 (1 * 10⁻³M) | 31.2 ± 2.5^{##} | 24.3 |
| 3 (1 * 10⁻³M) | 42.3 ± 2.4 | −2.7 |
| 4 (1 * 10⁻³M) | 19.9 ± 1.9^{##} | 51.7 |
| 5 (1 * 10⁻³M) | 18.8 ± 1.3^{##} | 54.4 |
| 6 (1 * 10⁻³M) | 28.5 ± 2.1^{##} | 30.8 |
| 7 (1 * 10⁻³M) | 15.4 ± 1.1^{##} | 62.6 |
| 8 (1 * 10⁻³M) | 27.1 ± 1.4^{##} | 34.2 |
| 9 (1 * 10⁻³M) | 31.7 ± 2.0^{##} | 23.1 |
| 10 (1 * 10⁻³M) | 18.1 ± 2.1^{##} | 56.1 |
| 11 (1 * 10⁻³M) | 8.1 ± 0.4^{##} | 80.3 |
| 12 (1 * 10⁻³M) | 20.4 ± 0.9 | 50.5 |
| 13 (1 * 10⁻³M) | 10.7 ± 1.5^{##} | 74.0 |
| 14 (1 * 10⁻³M) | 27.2 ± 2.7^{##} | 34.0 |
| 15 (1 * 10⁻³M) | 6.8 ± 0.5^{##} | 83.5 |
| 16 (1 * 10⁻³M) | 21.6 ± 1.9^{##} | 47.6 |
| 17 (1 * 10⁻³M) | 11.1 ± 1.3^{##} | 73.1 |
| 18 (1 * 10⁻³M) | 8.4 ± 1.7^{##} | 79.6 |
| 19 (1 * 10⁻³M) | 14.4 ± 2.2^{##} | 65.0 |
| 20 (1 * 10⁻³M) | 14.8 ± 1.4^{##} | 64.1 |
| 21 (1 * 10⁻³M) | 21.0 ± 1.6^{##} | 49.0 |
| 22 (1 * 10⁻³M) | 31.6 ± 2.7^{##} | 23.3 |
| 23 (1 * 10⁻³M) | 42.9 ± 2.9 | −4.1 |
| 24 (1 * 10⁻³M) | 20.7 ± 1.9^{##} | 49.8 |
| 25 (1 * 10⁻³M) | 19.5 ± 1.3^{##} | 52.7 |
| 26 (1 * 10⁻³M) | 29.1 ± 2.0^{##} | 29.4 |
| 27 (1 * 10⁻³M) | 15.8 ± 1.6^{##} | 61.7 |
| 28 (1 * 10⁻³M) | 27.3 ± 1.1^{##} | 33.7 |
| 29 (1 * 10⁻³M) | 31.5 ± 2.2^{##} | 23.5 |
| 30 (1 * 10⁻³M) | 17.7 ± 2.1^{##} | 57.0 |
| 31 (1 * 10⁻³M) | 8.8 ± 0.4^{##} | 78.0 |
| 32 (1 * 10⁻³M) | 19.6 ± 0.7^{##} | 52.4 |
| 33 (1 * 10⁻³M) | 9.9 ± 1.1^{##} | 76.0 |
| 34 (1 * 10⁻³M) | 26.6 ± 2.9^{##} | 35.4 |
| 35 (1 * 10⁻³M) | 6.0 ± 0.5^{##} | 85.4 |
| 36 (1 * 10⁻³M) | 20.8 ± 1.7^{##} | 49.5 |
| 37 (1 * 10⁻³M) | 10.5 ± 1.0^{##} | 74.5 |
| 38 (1 * 10⁻³M) | 8.0 ± 1.9^{##} | 80.6 |
| SSL1-IM8 (1 * 10⁻³M) | 22.7 ± 2.4^{##} | 44.7 |
| SSL6-IM2 (1 * 10⁻³M) | 20.1 ± 1.1^{##} | 50.2 |
| SSL7-IM2 (1 * 10⁻³M) | 9.3 ± 0.5^{##} | 77.2 |
| SSL8-IM2 (1 * 10⁻³M) | 21.2 ± 1.2^{##} | 48.8 |
| SSL9-IM2 (1 * 10⁻³M) | 27.6 ± 2.7^{##} | 32.8 |
| SSL10-IM7 (1 * 10⁻³ | 16.4 ± 2.1^{##} | 60.9 |
| M) | | |
| SSL11-IM2 (1 * 10⁻³ | 10.0 ± 1.4^{##} | 75.5 |
| M) | | |
| Ozagrel (1 * 10⁻⁴M) | 34.8 ± 3.1 | 15.5 |
| 1 (1 * 10⁻⁴M) | 35.2 ± 4.3 | 14.6 |
| 2 (1 * 10⁻⁴M) | 33.1 ± 0.3 | 19.7 |
| 3 (1 * 10⁻⁴M) | 46.0 ± 5.0 | −11.7 |
| 4 (1 * 10⁻⁴M) | 25.9 ± 4.0^{##} | 37.1 |
| 5 (1 * 10⁻⁴M) | 29.5 ± 0.9^{##} | 28.4 |
| 6 (1 * 10⁻⁴M) | 32.6 ± 0.9 | 20.9 |
| 7 (1 * 10⁻⁴M) | 27.7 ± 2.3^{##} | 32.8 |
| 8 (1 * 10⁻⁴M) | 28.4 ± 3.8^{##} | 31.1 |
| 9 (1 * 10⁻⁴M) | 45.4 ± 2.9 | −10.2 |
| 10 (1 * 10⁻⁴M) | 34.8 ± 4.7 | 15.5 |
| 11 (1 * 10⁻⁴M) | 18.9 ± 1.8^{##} | 54.1 |
| 12 (1 * 10⁻⁴M) | 40.2 ± 1.5 | 2.4 |
| 13 (1 * 10⁻⁴M) | 28.6 ± 1.8^{##} | 30.6 |
| 14 (1 * 10⁻⁴M) | 29.4 ± 2.1^{##} | 28.6 |
| 15 (1 * 10⁻⁴M) | 27.9 ± 1.2^{##} | 32.3 |
| 16 (1 * 10⁻⁴M) | 34.3 ± 4.0 | 16.7 |
| 17 (1 * 10⁻⁴M) | 24.8 ± 4.1^{##} | 39.8 |
| 18 (1 * 10⁻⁴M) | 26.0 ± 3.7^{##} | 36.9 |
| 19 (1 * 10⁻⁴M) | 23.0 ± 2.1^{##} | 44.2 |
| 20 (1 * 10⁻⁴M) | 28.6 ± 0.3^{##} | 30.6 |
| 21 (1 * 10⁻⁴M) | 35.4 ± 4.4 | 14.1 |
| 22 (1 * 10⁻⁴M) | 33.5 ± 0.9 | 18.7 |
| 23 (1 * 10⁻⁴M) | 46.6 ± 3.2 | −13.1 |
| 24 (1 * 10⁻⁴M) | 26.7 ± 4.1^{##} | 35.2 |
| 25 (1 * 10⁻⁴M) | 30.3 ± 1.9^{##} | 26.5 |
| 26 (1 * 10⁻⁴M) | 33.2 ± 0.7 | 19.4 |
| 27 (1 * 10⁻⁴M) | 28.1 ± 1.9^{##} | 31.8 |
| 28 (1 * 10⁻⁴M) | 28.6 ± 3.8^{##} | 30.6 |
| 29 (1 * 10⁻⁴M) | 45.2 ± 3.9 | −9.7 |
| 30 (1 * 10⁻⁴M) | 34.4 ± 4.5 | 16.5 |
| 31 (1 * 10⁻⁴M) | 18.3 ± 1.2^{##} | 55.6 |
| 32 (1 * 10⁻⁴M) | 39.4 ± 1.4 | 4.4 |
| 33 (1 * 10⁻⁴M) | 27.8 ± 1.1^{##} | 32.5 |
| 34 (1 * 10⁻⁴M) | 28.8 ± 2.8^{##} | 30.1 |
| 35 (1 * 10⁻⁴M) | 27.5 ± 1.9^{##} | 33.3 |
| 36 (1 * 10⁻⁴M) | 33.9 ± 4.2 | 17.7 |
| 37 (1 * 10⁻⁴M) | 24.2 ± 2.1^{##} | 41.3 |
| 38 (1 * 10⁻⁴M) | 26.2 ± 3.3^{##} | 36.4 |
| SSL6-IM2 (1 * 10⁻⁴M) | 27.1 ± 0.9 | 22.1 |
| SSL7-IM2 (1 * 10⁻⁴M) | 23.7 ± 2.0^{##} | 31.9 |
| SSL8-IM2 (1 * 10⁻⁴M) | 25.1 ± 1.8^{##} | 27.9 |
| SSL16-IM2 (1 * 10⁻⁴M) | 29.1 ± 4.0 | 16.4 |
| SSL17-IM2 (1 * 10⁻⁴M) | 20.4 ± 2.1^{##} | 41.4 |
| SSL18-IM2 (1 * 10⁻⁴M) | 23.7 ± 1.9^{##} | 31.9 |
| SSL19-IM3 (1 * 10⁻⁴M) | 21.8 ± 2.0^{##} | 37.4 |

**P < 0.01 vs. blank control group;
^{##}P < 0.01 vs. AA model group

### 1.6 Conclusion

Under the conditions of this experiment, among the 38 test compounds, all compounds except for compound 3 and Compound 23 can significantly inhibit AA-induced platelet aggregation at a concentration of 1*10-3 M. Compound 4, compound 5, compound 7, compound 8, compound 11, compound 13, compound 14, compound 15, compound 17, compound 18, compound 19, compound 20, compound 24, compound 25, compound 27, compound 28, compound 31, compound 33, compound 34, compound 35, compound 37, and compound 38 can significantly inhibit AA-induced platelet aggregation at a concentration of 1*10⁻⁴ M.

### 2. Experimental Method 2

### 2.1 Grouping

Several experimental groups were established, including a blank control group, an AA model control group, and various test compounds. Each test compound had three concentration groups: 10⁻³ M, 10⁻⁴ M, and 10⁻⁵ M.

### 2.2 Preparation of Rabbit Test Plasma

Rabbits were anesthetized with a 20% urethane solution (5 mL/kg body weight) through intraperitoneal injection. After being fixed in a supine position, the skin of the neck was cut about 6 cm, bluntly separated with a hemostat, and the trachea was exposed after the common carotid artery was separated. The distal end of the common carotid artery was ligated with a fine thread, and the proximal end was clamped with an arterial clamp for vascular intubation. After the intubation was fixed, the artery clamp was opened, and the blood was placed into a blood collection tube containing 3.8% trisodium citrate so that the blood was mixed well with the 3.8% trisodium citrate in a volume ratio of 9:1.

The prepared anticoagulant blood was mixed well and centrifuged for 10 minutes at 500 rpm, and the upper layer of plasma was aspirated, then platelet-rich plasma (PRP) was obtained, and the remaining anticoagulant blood was centrifuged for 15 minutes at 3,000 rpm, followed by aspirating the supernatant to obtain platelet-poor plasma (PPP).

### 2.3 Inhibitory Effect of the Test Compound on AA-Induced Platelet Aggregation in Rabbits In Vitro

First, 300 μL of PPP was added to the test cup and put into the test wells, and calibrated by pressing the “PPP” key. Subsequently, 240 μL of the washed platelets were added to the test cup, then added with 30 μL of different concentrations of drug solution, incubated for 10 minutes at 37° C. in a preheated bath, and then put into the test wells. 3 μL of 100× CaCl₂) solution (final concentration of 0.2 g/L) and 30 μL of inducer (final concentration of AA of 80 M) were added to the test wells, and then the “Start” key was pressed immediately. The maximum aggregation rate within 4 minutes was measured. Measurements were repeated five times for each drug concentration.

The platelet aggregation inhibition rate was calculated with the following formula:

Inhibition rate (%)=(maximum aggregation rate in the AA model control group−maximum aggregation rate in the administration group)/maximum aggregation rate in the AA model control group×100%

### 2.4 Statistical Method

All data were expressed as mean standard deviation (mean b SD). SPSS 22.0 software was used for statistical analysis of the data. Intergroup data were analyzed using one-way ANOVA, with P<0.05 considered as statistically significant and P<0.01 considered as highly significant.

### 2.5 Experimental Results (1)

TABLE 2
Effect of samples on AA-induced platelet aggregation in
rabbits (mean ± SD, n = 5)

| Group | Aggregation rate | Inhibition rate (%) |
|---|---|---|
| Blank control group | 3.5 ± 2.1 | — |
| AA model group | 39.8 ± 3.4** | — |
| Ozagrel (1 * 10⁻³M) | 22.2 ± 5.1^{##} | 44.3 |
| Ozagrel (1 * 10⁻⁴M) | 27.0 ± 2.6^{#} | 32.3 |
| Ozagrel (1 * 10⁻⁵M) | 34.6 ± 4.2 | 13.1 |
| 39 (1 * 10⁻³M) | 25.8 ± 6.1^{##} | 35.1 |
| 39 (1 * 10⁻⁴M) | 33.3 ± 6.3 | 16.4 |
| 39 (1 * 10⁻⁵M) | 38.3 ± 5.6 | 3.8 |
| 40 (1 * 10⁻³M) | 25.5 ± 2.7^{##} | 36.0 |
| 40 (1 * 10⁻⁴M) | 29.6 ± 3.5 | 25.6 |
| 40 (1 * 10⁻⁵M) | 39.2 ± 5.1 | 1.5 |
| 41 (1 * 10⁻³M) | 26.9 ± 3.6^{#} | 32.4 |
| 41 (1 * 10⁻⁴M) | 36.9 ± 4.1 | 7.4 |
| 41 (1 * 10⁻⁵M) | 37.7 ± 2.1 | 5.2 |
| 42 (1 * 10⁻³M) | 22.0 ± 2.9^{##} | 44.8 |
| 42 (1 * 10⁻⁴M) | 38.0 ± 4.5 | 4.4 |
| 42 (1 * 10⁻⁵M) | 38.4 ± 5.0 | 3.6 |
| 43 (1 * 10⁻³M) | 24.7 ± 6.7^{##} | 37.9 |
| 43 (1 * 10⁻⁴M) | 36.2 ± 4.7 | 9.1 |
| 43 (1 * 10⁻⁵M) | 40.6 ± 5.0 | −2.0 |
| 44 (1 * 10⁻³M) | 27.2 ± 2.9^{#} | 31.8 |
| 44 (1 * 10⁻⁴M) | 33.8 ± 4.8 | 15.1 |
| 44 (1 * 10⁻⁵M) | 39.7 ± 2.7 | 0.3 |
| 45 (1 * 10⁻³M) | 26.3 ± 4.2^{#} | 34.0 |
| 45 (1 * 10⁻⁴M) | 37.9 ± 3.4 | 4.9 |
| 45 (1 * 10⁻⁵M) | 40.2 ± 4.2 | −1.1 |
| 46 (1 * 10⁻³M) | 22.4 ± 3.4^{##} | 43.6 |
| 46 (1 * 10⁻⁴M) | 36.6 ± 4.1 | 8.1 |
| 46 (1 * 10⁻⁵M) | 38.1 ± 2.9 | 4.3 |
| 47 (1 * 10⁻³M) | 24.5 ± 3.9^{##} | 38.4 |
| 47 (1 * 10⁻⁴M) | 37.3 ± 3.5 | 6.3 |
| 47 (1 * 10⁻⁵M) | 41.6 ± 1.0 | −4.6 |
| 48 (1 * 10⁻³M) | 37.2 ± 4.4 | 6.6 |
| 48 (1 * 10⁻⁴M) | 36.3 ± 4.2 | 8.8 |
| 48 (1 * 10⁻⁵M) | 36.1 ± 2.6 | 9.2 |
| 49 (1 * 10⁻³M) | 24.7 ± 3.9^{##} | 37.9 |
| 49 (1 * 10⁻⁴M) | 34.9 ± 7.9 | 12.4 |
| 49 (1 * 10⁻⁵M) | 40.0 ± 3.7 | −0.6 |
| 50 (1 * 10⁻³M) | 23.3 ± 4.3^{##} | 41.4 |
| 50 (1 * 10⁻⁴M) | 36.7 ± 3.9 | 7.7 |
| 50 (1 * 10⁻⁵M) | 36.3 ± 4.6 | 8.9 |
| 51 (1 * 10⁻³M) | 26.7 ± 8.4^{#} | 32.9 |
| 51 (1 * 10⁻⁴M) | 35.1 ± 1.6 | 11.8 |
| 51 (1 * 10⁻⁵M) | 36.9 ± 5.0 | 7.2 |

**P < 0.01 vs. blank control group;
^{#}P < 0.05;
^{##}P < 0.01 vs. AA model group

### 2.6 Conclusion (1)

Under the conditions of this experiment, among the 13 test compounds, all compounds except for compound 48 can significantly inhibit AA-induced platelet aggregation at a concentration of 1*10⁻³ M.

### 2.7 Experimental Results (2)

TABLE 3
Effect of samples on AA-induced platelet aggregation
in rabbits (mean ± SD, n = 5)

| Group | Aggregation rate | Inhibition rate (%) |
|---|---|---|
| Blank control group | 3.6 ± 2.3 | — |
| AA model group | 54.0 ± 5.1** | — |
| Ozagrel (1 * 10⁻³M) | 24.5 ± 5.2^{##} | 54.6 |
| Ozagrel (1 * 10⁻⁴M) | 31.4 ± 4.7^{##} | 41.9 |
| Ozagrel (1 * 10⁻⁵M) | 45.6 ± 4.4 | 15.6 |
| 52 (1 * 10⁻³M) | 30.9 ± 5.5^{##} | 42.7 |
| 52 (1 * 10⁻⁴M) | 40.0 ± 6.7 | 26.0 |
| 52 (1 * 10⁻⁵M) | 42.4 ± 10.7 | 21.5 |
| 53 (1 * 10⁻³M) | 49.3 ± 5.1 | 8.7 |
| 53 (1 * 10⁻⁴M) | 48.0 ± 7.7 | 11.1 |
| 53 (1 * 10⁻⁵M) | 51.3 ± 12.2 | 5.1 |
| 54 (1 * 10⁻³M) | 32.0 ± 5.3^{##} | 40.8 |
| 54 (1 * 10⁻⁴M) | 41.4 ± 7.0 | 23.3 |
| 54 (1 * 10⁻⁵M) | 49.0 ± 7.2 | 9.3 |
| 55 (1 * 10⁻³M) | 22.1 ± 7.0^{##} | 59.1 |
| 55 (1 * 10⁻⁴M) | 46.1 ± 9.9 | 14.7 |
| 55 (1 * 10⁻⁵M) | 45.1 ± 4.4 | 16.5 |
| 56 (1 * 10⁻³M) | 8.1 ± 4.0^{##} | 85.0 |
| 56 (1 * 10⁻⁴M) | 25.8 ± 7.0^{##} | 52.2 |
| 56 (1 * 10⁻⁵M) | 45.8 ± 4.6 | 15.1 |
| 57 (1 * 10⁻³M) | 15.4 ± 5.0^{##} | 71.6 |
| 57 (1 * 10⁻⁴M) | 38.8 ± 12.0 | 28.1 |
| 57 (1 * 10⁻⁵M) | 43.0 ± 11.6 | 20.4 |
| 58 (1 * 10⁻³M) | 42.6 ± 7.9 | 21.2 |
| 58 (1 * 10⁻⁴M) | 43.6 ± 5.2 | 19.2 |
| 58 (1 * 10⁻⁵M) | 49.7 ± 7.0 | 8.0 |
| 59 (1 * 10⁻³M) | 12.9 ± 4.0^{##} | 76.1 |
| 59 (1 * 10⁻⁴M) | 35.1 ± 9.9^{#} | 35.0 |
| 59 (1 * 10⁻⁵M) | 46.8 ± 3.2 | 13.3 |

**P < 0.01 vs. blank control group;
^{#}P < 0.05;
^{##}P < 0.01 vs. AA model group

### 2.8 Conclusion (2)

Under the conditions of this experiment, among the 8 test compounds, all compounds except for compound 53 and Compound 58 can significantly inhibit AA-induced platelet aggregation at a concentration of 1*10-3 M. Compound 56 and Compound 59 can significantly inhibit AA-induced platelet aggregation at a concentration of 1*10⁻⁴ M.

Effect Example 2: Experimental study on the effects of therapeutic administration (i.v.) of the test compound on cerebral ischemic injury caused by middle cerebral artery occlusion/reperfusion in the rat brain

### I. Experimental Method

### 1. Establishment of the Middle Cerebral Artery Occlusion/Reperfusion (MCAO/R) Model in Rats

Male SD rats weighing 250 to 300 g were taken, and the rats in each group fasted for 12 hours before modeling, while water was not restricted. According to the method of Longa et al.^{[1]}, a rat model of MCAO/R was established by occluding the blood flow of internal carotid artery using the thread embolization method. The rats were anesthetized with 3% chloral hydrate (300 mg/kg, 1 mL/100 g body weight) through intraperitoneal injection and fixed in the supine position on the operating table. A midline incision was made in the neck, and the right common carotid artery was isolated using forceps and threaded for later use. The external and internal carotid arteries were isolated from the bifurcation of the common carotid artery. A single thread was threaded through the internal carotid artery, and two threads were threaded through the external carotid artery. Both distal and proximal ends were ligated, and the middle part of the ligation site was cut using ophthalmic scissors. The proximal main trunk of the external carotid artery was isolated and reserved for later use. The prepared cotton thread was used to half-ligate the common carotid artery (tying a slipknot), and the thread prepared for the internal carotid artery was tightened with hemostatic forceps, temporarily occluding the blood flow of the internal carotid artery. A small incision at the proximal main trunk of the external carotid artery was made using ophthalmic scissors, and then, the fishing line was held with straight forceps, inserted from this incision, and slowly advanced through the isolated proximal main trunk of the external carotid artery towards the intracranial direction of the internal carotid artery. The advancement was stopped at a predetermined position, and then the thread of the internal carotid artery that was tightened with the hemostatic forceps was loosened. The predetermined position referred to the point where, starting from the bifurcation of the common carotid artery and advancing about 18 mm, resistance was encountered, indicating that all blood supply to the Middle Cerebral Artery (MCA) has been occluded. Another thread was threaded and tightened around the proximal main trunk of the external carotid artery and the fishing line that had already been inserted into the predetermined position. The half-ligated thread on the common carotid artery was then loosened, and the skin was subsequently sutured. After 2 hours of ischemia, a small section of the fishing line was pulled out. If the rat showed intense struggling or twisting, it was considered a successful reperfusion. After rats in the blank control group were anesthetized, only the bifurcation of the internal and external carotid arteries was exposed without the occlusion of the MCA.

### 2. Animal Grouping and Administration

Successfully modeled rats (neurological function score of 3 after reperfusion) were taken and divided into 20 groups (8 rats for each) according to the random number table method. The groups were divided into a model control group, three dose groups (high, 12 mg/kg, 2.4 mg/mL; medium, 6 mg/kg, 1.2 mg/mL; low, 3 mg/kg, 0.6 mg/mL) for each of the six test compounds, and a positive drug Ozagrel group (6 mg/kg, 1.2 mg/mL), respectively. Both blank and model control groups were administered equal volumes of mixed solvents, and the rats in each group were administered by tail vein injection (i.v.) 2 hours after reperfusion, once a day for 3 consecutive days, with a volume of 0.5 mL/100 g body weight.

### 3. Effect of Therapeutic Administration on the Neurological Function Score in MCAO/R Rats

Ten minutes after the last administration, the neurological function of the animals was graded according to the modified Bederson scoring method. The standard is as follows[2]:
0 point: no neurological symptoms;
1 point: when the rat is suspended by lifting its tail, the forelimb on the surgical side of the rat was flexed and pressed against the chest wall;
2 points: on a smooth plane, when the rat is pushed from the surgical side to the opposite side, the resistance is less than when it is moved to the same side;
3 points: the animal moves around or in circles when moving freely;
4 points: flaccid paralysis, no spontaneous movement of limbs.

### 4. Effect of Therapeutic Administration on Cerebral Infarction Rate and Brain Water Content in MCAO/R Rats

After Bederson scoring, the rats were executed by cervical dislocation, and the whole brain was removed and weighed. After weighing, the whole brain was placed in a −20° C. refrigerator for 20 minutes. Four coronal cuts were made at the optic chiasm and 2 mm before and after it. The five cut brain slices were immersed in a phosphate buffer solution containing 1% TTC, and incubated in a 37° C. water bath in the dark for 15 minutes. After 15 minutes of incubation, the brain slices were taken out, arranged in order, and photographed with a digital camera. The pale area (infarct area) and the non-pale area (normal area) were separated, weighed, and recorded as the weight of the pale area and the weight of the non-pale area, respectively. The sum of the two weights was recorded as the wet weight of the brain tissue. The infarct percentage is calculated as follows^{[3]}:

Infarct percentage(%)=weight of pale area/(weight of pale area+weight of non-pale area)×100%

The stained brain tissues were dried for 24 hours at 110° C. in an oven, weighed, and recorded as the dry weight of the brain tissues. The brain water content was calculated by comparing the dry weight of the brain with the wet weight of the brain^{[4]}:

Brain tissue water content(%)=(1−dry weight of brain tissue/wet weight of brain tissue)×100%.

### 5. Statistical Method

All data were expressed as Mean±SD and statistically analyzed using IBM SPSS Statistics v22.0 software, and intergroup data were analyzed using one-way ANOVA. P<0.05 is considered as statistically significant and P<0.01 is considered as highly significant. Data results were plotted using Graphpad Prism 5.0 software. (Note: The blank control group is not included in the statistical test of neurological function scores and cerebral infarction area).

### II. Experimental Results

TABLE 4
Effect of the test compound on relevant indicators after cerebral ischemia in
rats (mean ± SD, n = 8)

| | | | | Cerebral | Brain water |
|---|---|---|---|---|---|
| | Neurological | Clotting time | Bleeding time | infarction rate | content |
| Group | function score | (s) | (s) | (%) | (%) |
| Blank control | 0 | 151.81 ± 27.96 | 325.75 ± 41.91 | 0 | 78.58 ± 1.24 |
| group | | | | | |
| Model group | 2.63 ± 0.52 | 115.13 ± 14.21 | 239.38 ± 37.97 | 33.77 ± 2.45 | 82.82 ± 1.47** |
| Ozagrel group | 1.50 ± 0.93 | 154.50 ± 16.91 | 300.00 ± 26.93 | 21.70 ± | 79.73 ± 2.02 |
| | | | | 4.69^{▴▴} | |
| 11 low dose | 2.25 ± 0.46 | 138.38 ± 27.34 | 307.63 ± 64.59 | 28.07 ± 4.88 | 82.04 ± 1.91* |
| 11 medium | 2.00 ± 0.53 | 136.63 ± 9.80 | 279.75 ± 37.97 | 19.96 ± | 80.33 ± 1.65 |
| dose | | | | 4.44^{▴▴} | |
| 11 high dose | 1.25 ± 0.46^{▴▴} | 152.81 ± 29.69 | 319.25 ± 49.38 | 13.53 ± | 80.58 ± 0.77 |
| | | | | 7.24^{▴▴} | |
| 13 low dose | 2.00 ± 0.53 | 146.94 ± 24.73 | 303.00 ± 39.59 | 23.02 ± | 80.87 ± 2.84 |
| | | | | 5.96^{▴▴} | |
| 13 medium | 1.88 ± 0.64 | 164.56 ± | 314.50 ± 41.83 | 22.15 ± | 80.58 ± 1.56 |
| dose | | 34.63^{▴} | | 4.56^{▴▴} | |
| 13 high dose | 1.50 ± 0.53 | 163.63 ± | 301.25 ± 46.40 | 14.30± | 80.73 ± 1.10 |
| | | 19.85^{▴} | | 4.87^{▴▴} | |
| 15 low dose | 2.38 ± 0.74 | 155.38 ± 41.85 | 281.38 ± 61.33 | 29.98 ± 5.52 | 81.71 ± 1.64 |
| 15 medium | 1.75 ± 0.71 | 156.50 ± 31.15 | 304.88 ± 43.01 | 21.57 ± | 81.19 ± 1.32 |
| dose | | | | 7.30^{▴▴} | |
| 15 high dose | 1.50 ± 0.53 | 163.13 ± 20.06 | 289.63 ± 60.00 | 15.57± | 80.84 ± 1.40 |
| | | | | 5.76^{▴▴} | |
| 17 low dose | 2.25 ± 0.71 | 141.25 ± 28.04 | 290.75 ± 79.23 | 30.61 ± 5.72 | 82.47 ± 3.03** |
| 17 medium | 1.88 ± 0.83 | 155.00 ± 29.81 | 307.50 ± 64.66 | 22.75 ± | 81.96 ± 2.16* |
| dose | | | | 4.61^{▴▴} | |
| 17 high dose | 1.63 ± 0.74 | 163.00 ± 29.03 | 338.38 ± | 17.37± | 80.34 ± 1.65 |
| | | | 45.43^{▴} | 5.92^{▴▴} | |
| 18 low dose | 2.25 ± 0.46 | 152.38 ± 28.67 | 267.38 ± 50.19 | 26.58 ± 4.57 | 80.64 ± 2.21 |
| 18 medium | 1.88 ± 0.83 | 158.19 ± 19.62 | 271.63 ± 40.55 | 20.75 ± | 80.95 ± 1.79 |
| dose | | | | 5.11^{▴▴} | |
| 18 high dose | 1.63 ± 0.52 | 147.25 ± 13.30 | 330.25 ± 50.14 | 17.78 ± | 80.54 ± 1.62 |
| | | | | 3.11^{▴▴} | |
| 19 low dose | 2.00 ± 0.53 | 136.25 ± 36.28 | 272.38 ± 52.89 | 28.23 ± 2.76 | 82.40 ± 1.50** |
| 19 medium | 2.00 ± 0.76 | 148.63 ± 28.41 | 274.13 ± 47.46 | 22.38 ± | 81.92 ± 1.66* |
| dose | | | | 5.97^{▴▴} | |
| 19 high dose | 1.75 ± 0.89 | 151.19 ± 21.35 | 304.75 ± 35.16 | 13.48 ± | 80.84 ± 1.04 |
| | | | | 1.39^{▴▴} | |

*P < 0.05,
**P < 0.01 vs. blank control group;
^{▴}P < 0.05,
^{▴▴}P < 0.01 vs. model group

### III. Experimental Conclusion

Under the conditions of this experiment, each test compound can significantly reduce the infarct area after cerebral ischemia-reperfusion injury in rats at medium and high doses. Low dose of 13 can also significantly reduce infarct size. High dose of 11 can significantly reduce neurological deficits in rats. Low and medium doses of 19 and 17, and low dose of 11 did not significantly improve cerebral edema in rats. Medium and high doses of 13 can significantly prolong the coagulation time of rats. The result shows that intravenous administration of the test drugs can improve MCAO/R-induced cerebral ischemic injury in rats, with high doses of 19 and 11 being more advantageous in reducing the infarct area (with the smallest mean infarct rate), and high dose of 11 being more advantageous in improving the neurological function scores of the rats.

Effect Example 3: Pharmacokinetic and blood-brain barrier (BBB) studies of compounds after single intravenous (IV) and oral (PO) administration in SD rats

### I. Experimental Method

### 1. Experimental Method of Gavage Administration in Rats

The experimental method of administration by gavage injection in rats: SD rats weighing 180 to 220 g were fasted for 12 hours prior to the experiment, with free access to water. After administration, food and water were withheld for 4 hours. After this 4-hour period, the rats were allowed free access to water, and food was provided after 8 hours. The drugs were administered at a set dose. N=3, blood samples were taken before administration, and 5 minutes, 0.25 hours, 0.5 hours, 0.75 hours, 1 hour, 2 hours, 4 hours, 8 hours, and 24 hours after administration, respectively, and placed into sodium heparin anticoagulant tubes. The tubes were centrifuged for 1 hour at 4° C. and at 8000 rpm for 5 minutes, and the plasma was then separated and stored in centrifuge tubes at −70° C. for analysis.

### 2. Experimental Method for Intravenous Administration to Rats

The experimental method of intravenous administration to rats: SD rats weighing 180 to 220 g were fasted for 12 hours prior to the experiment, with free access to water. After administration, food and water were withheld for 4 hours. After this 4-hour period, the rats were allowed free access to water, and food was provided after 8 hours. The drugs were administered at a set dose. N=3, blood samples were taken before administration, and 5 minutes, 0.25 hours, 0.5 hours, 0.75 hours, 1 hour, 2 hours, 4 hours, 8 hours, and 24 hours after administration, respectively, and placed into sodium heparin anticoagulant tubes. The tubes were centrifuged for 1 hour at 4° C. and at 8000 rpm for 5 minutes, and the plasma was then separated and stored in centrifuge tubes at −70° C. for analysis.

### 3. Data Processing

The plasma concentration-time data were processed using the Phoenix® WinNonlin® 8.0 program to calculate pharmacokinetic parameters.

Among them, Cmax and Tmax are measured values, the elimination rate constant k of the tail section of the C-t curve is obtained from the linear regression of LnC-t, and the value of AUC₀₋₄ is calculated by the trapezoidal area method, and the area under the curve from 0 to ∞ time is AUC=AUC₀₋₄+Ct/k.

### 4. Chromatography and Mass Spectrometry Methods for Sample Determination

TABLE 5
Summary of chromatography and mass spectrometry methods

| Chromatography system | ACQUITY UPLC I-Class Plus | | |
|---|---|---|---|
| Chromatography column | ACQUITY UPLC BEH C18, | | |
| | (1.7 μm, 2.1 × 50 mm) | | |
| Column temperature | 40° C. | | |
| Mobile phase | A: 0.1% formic acid-water (v/v) B: acetonitrile | | |
| Flow rate | 400 μL/minute | | |
| Operation time | 4 minutes | | |
| | Time (minute) | % A | % B |
| Elution gradient | 0.01 | 95 | 5 |
| | 0.50 | 95 | 5 |
| | 2.0 | 50 | 50 |
| | 2.5 | 5 | 95 |
| | 3.2 | 5 | 95 |
| | 3.3 | 95 | 5 |
| | 4.0 | 95 | 5 |

| | |
|---|---|
| Mass spectrometry system | Xevo TQ-XS |
| Detection method | ESI+, MRM |
| Capillary (kV) | 2.7 |
| Desolvation Temp (° C.) | 450 |
| Desolvation Gas (L/Hr) | 1000 |

### II. Experimental Results

TABLE 6

| | Ozagrel (rat- | 11 (rat- | 15 (rat- | 17 (rat- | 18 (rat- | 19 (rat- |
|---|---|---|---|---|---|---|
| Animal code | Plasma) | Plasma) | Plasma) | Plasma) | Plasma) | Plasma) |
| Route of | IV | IV | IV | IV | IV | IV |
| Dosing | | | | | | |
| Dose Level | 1 | 1 | 1 | 1 | 1 | 1 |
| (mg/kg) | | | | | | |
| T_{1/2} (h) | 0.26 | 34.78 | 0.91 | 3.24 | 10.36 | 0.44 |
| Tₘₐₓ (h) | 0.083 | 0.083 | 0.083 | 0.083 | 0.083 | 0.083 |
| Cₘₐₓ (ng/mL) | 1839.57 | 5139.30 | 3161.04 | 6838.08 | 6337.49 | 5486.65 |
| AUC₍₀₋ₜ₎ | 509.87 | 86369.45 | 3430.93 | 17542.09 | 55887.37 | 3248.01 |
| (h *ng/mL) | | | | | | |
| AUC_{(0-∞)} | 510.28 | 228137.06 | 3439.27 | 17615.89 | 70742.72 | 3254.4 |
| (h*ng/mL) | | | | | | |
| MRT₍₀₋ₜ₎ (h) | 0.12 | 10.54 | 1.14 | 3.80 | 7.73 | 0.50 |
| MRT_{(0-∞)} (h) | 0.13 | 50.08 | 1.16 | 3.91 | 14.28 | 0.51 |
| C₀ (ng/mL) | 3672.18 | 5147.06 | 3709.43 | 7318.02 | 6586.83 | 6868.77 |
| Vₛₛ (L/kg) | 0.25 | 0.22 | 0.34 | 0.22 | 0.20 | 0.16 |
| V_{z} (L/kg) | 0.74 | 0.22 | 0.38 | 0.27 | 0.21 | 0.20 |

TABLE 7

| | Ozagrel (rat- | 11 (rat- | 15 (rat- | 17 (rat- | 18 (rat- | 19 (rat- |
|---|---|---|---|---|---|---|
| Group | Plasma) | Plasma) | Plasma) | Plasma) | Plasma) | Plasma) |
| Route of | PO | PO | PO | PO | PO | PO |
| Dosing | | | | | | |
| Dose Level | 6 | 6 | 6 | 6 | 6 | 6 |
| (mg/kg) | | | | | | |
| T_{1/2} (h) | 0.73 | 37.02 | 1.51 | 2.96 | 11.57 | 0.72 |
| Tmax (h) | 0.25 | 4.00 | 1.00 | 2.00 | 1.00 | 0.25 |
| Cmax | 3784.42 | 32404.85 | 18637.32 | 27174.41 | 41430.35 | 27342.27 |
| (ng/mL) | | | | | | |
| AUC₍₀₋ₜ₎ | 2677.19 | 586643.45 | 78397.61 | 178068.52 | 576281.73 | 36600.96 |
| (h*ng/mL) | | | | | | |
| AUC_{(0-∞)} | 2678.81 | 1,695,577.49 | 80739.43 | 178730.98 | 757250.44 | 36618.63 |
| (h*ng/mL) | | | | | | |
| MRT₍₀₋ₜ₎ | 0.62 | 10.97 | 2.57 | 4.16 | 8.39 | 1.19 |
| (h) | | | | | | |
| MRT_{(0-∞)} | 0.63 | 54.42 | 2.79 | 4.25 | 16.11 | 1.20 |
| (h) | | | | | | |
| F (%) | 87.51 | 113.20 | 380.84 | 169.18 | 171.86 | 187.81 |

TABLE 8

| | Ozagrel | | | | | |
|---|---|---|---|---|---|---|
| | (rat- | 11 (rat- | 15 (rat- | 17 (rat- | 18 (rat- | 19 (rat- |
| Group | Brain) | Brain) | Brain) | Brain) | Brain) | Brain) |
| Route of | IV | IV | IV | IV | IV | IV |
| Dosing | | | | | | |
| Dose Level | 1 | 1 | 1 | 1 | 1 | 1 |
| (mg/kg) | | | | | | |
| T_{1/2} (h) | 0.11 | 19.50 | 2.20 | 3.72 | 8.70 | 0.38 |
| Tmax (h) | 0.08 | 1.00 | 0.08 | 0.08 | 0.08 | 0.08 |
| Cmax | 27.55 | 57.55 | 41.21 | 84.09 | 75.54 | 36.00 |
| (ng/g) | | | | | | |
| AUC₍₀₋ₜ₎ | 4.96 | 855.25 | 33.91 | 169.74 | 633.23 | 16.44 |
| (h*ng/g) | | | | | | |
| AUC_{(0-∞)} | 5.25 | 1475.23 | 69.74 | 212.39 | 741.29 | 19.83 |
| (h*ng/g) | | | | | | |
| MRT₍₀₋ₜ₎ | 0.15 | 9.49 | 0.82 | 2.53 | 7.19 | 0.35 |
| (h) | | | | | | |
| MRT_{(0-∞)} | 0.17 | 27.41 | 3.05 | 4.70 | 11.47 | 0.56 |
| (h) | | | | | | |

TABLE 9

| | Ozagrel | | | | | |
|---|---|---|---|---|---|---|
| | (rat- | 11 (rat- | 15 (rat- | 17 (rat- | 18 (rat- | 19 (rat- |
| Group | Brain) | Brain) | Brain) | Brain) | Brain) | Brain) |
| Route of | PO | PO | PO | PO | PO | PO |
| Dosing | | | | | | |
| Dose Level | 6 | 6 | 6 | 6 | 6 | 6 |
| (mg/kg) | | | | | | |
| T_{1/2} (h) | 0.40 | 19.69 | 1.70 | 2.82 | 8.36 | 0.77 |
| Tmax (h) | 0.25 | 4.00 | 1.00 | 2.00 | 1.00 | 0.25 |
| Cmax | 42.57 | 384.75 | 198.69 | 323.13 | 459.57 | 167.92 |
| (ng/g) | | | | | | |
| AUC₍₀₋ₜ₎ | 35.86 | 7014.55 | 815.87 | 2329.55 | 6454.78 | 289.35 |
| (h*ng/g) | | | | | | |
| AUC_{(0-∞)} | 37.46 | 12,439.74 | 853.63 | 2336.46 | 7477.48 | 296.91 |
| (h*ng/g) | | | | | | |
| MRT₍₀₋ₜ₎ | 0.59 | 9.94 | 2.56 | 4.53 | 7.84 | 1.22 |
| (h) | | | | | | |
| MRT_{(0-∞)} | 0.68 | 28.46 | 2.91 | 4.60 | 11.70 | 1.32 |
| (h) | | | | | | |

### III. EXPERIMENTAL CONCLUSION

When five samples of Ozagrel, 11, 15, 17, 18, and 19 were administered intravenously at 1 mg/kg, the T_{1/2} and AUC_{(0-∞)} of 11, 15, 17, and 18 were significantly increased in the whole body blood circulation system as compared to that of Ozagrel, suggesting that the metabolic stability of the new compounds is more superior. In the brain tissue, the T_{1/2} and AUC_{(0-∞)} of 11, 15, 17, and 18 were significantly increased, indicating that the new compounds are more abundant in the brain tissue, which is more conducive to exerting better efficacy.

When five samples of Ozagrel, 11, 15, 17, 18, and 19 were administered orally at 6 mg/kg, the T_{1/2} and AUC_{(0-∞)} of 11, 15, 17, and 18 were significantly increased in the whole body blood circulation system as compared to that of Ozagrel, suggesting that the metabolic stability of the new compounds is more superior. In the brain tissue, the T_{1/2} and AUC_{(0-∞)} of 11, 15, 17, and 18 were significantly increased, indicating that the new compounds are more abundant in the brain tissue, which is more conducive to exerting better efficacy.

1. An imidazole compound of formula I or a pharmaceutically acceptable salt thereof; 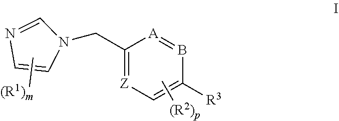
wherein A, B, and Z are independently CH or N;
R¹ and R² are each independently H, halogen, or C₁₋₆ alkyl;
m is 0, 1, 2, or 3;
R³ is 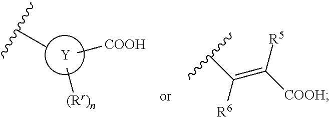
ring Y is a 3- to 6-membered cycloalkyl ring or a 3- to 6-membered heterocycloalkyl ring containing 1 to 3 heteroatoms selected from one or more than one of N, O, and S;
p and n are independently 0, 1, 2, 3, or 4;
each R is independently H, —OH, halogen, C₁₋₆ alkyl, or C₁₋₆ alkoxy;
R⁵ and R⁶ are independently H, C₂₋₆ alkenyl, C₂₋₆ alkynyl, C₂₋₆ alkynyl substituted by one or more than one R⁵⁻¹, 5- to 6-membered heteroaryl, or 5- to 6-membered heteroaryl substituted by one or more than one R⁵⁻², and the 5- to 6-membered heteroaryl in the 5- to 6-membered heteroaryl and 5- to 6-membered heteroaryl substituted by one or more than one R⁵⁻² contains 1 to 4 heteroatoms selected from one or more than one of N, O, or S; when there is more than one substituent, the substituents are the same or different;
R⁵⁻¹ and R⁵⁻² are independently halogen, C₁₋₆ alkyl, or C₁₋₆ alkoxy;
when A, B, and Z are CH at the same time, R⁵ and R⁶ are not H at the same time.

2. The imidazole compound of formula I or the pharmaceutically acceptable salt thereof according to claim 1, wherein the imidazole compound of formula I satisfies one or more than one of the following conditions:
(1) A, B, and Z are all CH, or at least one selected from the group of A, B, and Z is N;
(2) R¹ is H or halogen;
(3) 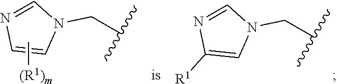
(4) m, p, and n are independently 0 or 1;
(5) when cis-trans isomerism of 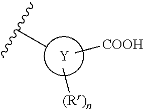
is present, the 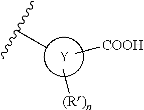
is of trans configuration;
(6) 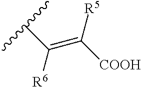
is of trans configuration;
(7) R^{r} is independently H or —OH;
(8) R⁵ is independently H, C₂₋₆ alkynyl substituted by one or more than one R⁵⁻¹, or 5- to 6-membered heteroaryl containing 1 to 2 N heteroatoms;
(9) R⁶ is H.

3. The imidazole compound of formula I or the pharmaceutically acceptable salt thereof according to claim 1, wherein the imidazole compound of formula I satisfies one or more than one of the following conditions:
(1) when R¹ and R² are independently halogen, the halogen is fluorine, chlorine, bromine, or iodine;
(2) when R¹ and R² are independently C₁₋₆ alkyl, the C₁₋₆ alkyl is methyl, ethyl, n-propyl, isopropyl, n-butyl, sec-butyl, isobutyl, or tert-butyl;
(3) when ring Y is a 3- to 6-membered cycloalkyl ring, the 3- to 6-membered cycloalkyl ring is a cyclopropyl, cyclobutyl, cyclopentyl, or cyclohexyl ring;
(4) when ring Y is a 3- to 6-membered heterocycloalkyl ring, the 3- to 6-membered heterocycloalkyl ring contains one N heteroatom;
(5) when R^{r} is independently C₁₋₆ alkyl, the C₁₋₆ alkyl is methyl, ethyl, n-propyl, isopropyl, n-butyl, sec-butyl, isobutyl, or tert-butyl;
(6) when R^{r} is independently C₁₋₆ alkoxy, the C₁₋₆ alkoxy is methoxy, ethoxy, n-propoxy, isopropoxy, n-butoxy, sec-butoxy, isobutoxy, or tert-butoxy;
(7) when R⁵ and R⁶ are independently C₂₋₆ alkenyl, the C₂₋₆ alkenyl is vinyl, propenyl, or allyl;
(8) when R⁵ and R⁶ are independently C₂₋₆ alkynyl or C₂₋₆ alkynyl substituted by one or more than one R⁵⁻¹, the C₂₋₆ alkynyl in the C₂₋₆ alkynyl and C₂₋₆ alkynyl substituted by one or more than one R⁵⁻¹ is ethynyl, propynyl, or propargyl;
(9) when R⁵ and R⁶ are independently 5- to 6-membered heteroaryl or 5- to 6-membered heteroaryl substituted by one or more than one R⁵⁻², the 5- to 6-membered heteroaryl in the 5- to 6-membered heteroaryl and the 5- to 6-membered heteroaryl substituted by one or more than one R⁵⁻² contains 1 to 2 N heteroatoms;
(10) when R⁵⁻¹ and R⁵⁻² are independently halogen, the halogen is fluorine, chlorine, bromine, or iodine;
(11) when R⁵⁻¹ and R⁵⁻² are independently C₁₋₆ alkyl, the C₁₋₆ alkyl is methyl, ethyl, n-propyl, isopropyl, n-butyl, sec-butyl, isobutyl, or tert-butyl;
(12) when R⁵⁻¹ and R⁵⁻² are independently C₁₋₆ alkoxy, the C₁₋₆ alkoxy is methoxy, ethoxy, n-propoxy, isopropoxy, n-butoxy, sec-butoxy, isobutoxy, or tert-butoxy; 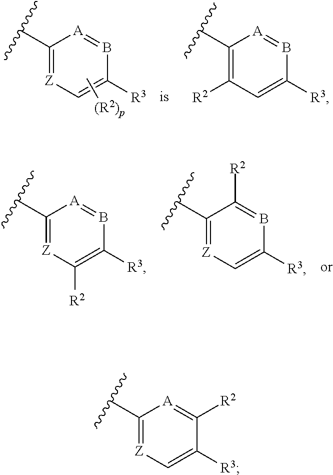
(14) when at least one selected from the group of A, B, and Z is N, 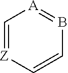
is a pyridine ring, a pyrimidine ring, or a pyridazine ring.

4. The imidazole compound of formula I or the pharmaceutically acceptable salt thereof according to claim 3, wherein the imidazole compound of formula I satisfies one or more than one of the following conditions:
(1) when R¹ and R² are independently halogen, the halogen is fluorine or chlorine;
(2) when ring Y is a 3- to 6-membered cycloalkyl ring, the 3- to 6-membered cycloalkyl ring is a cyclopropyl or cyclobutyl ring;
(3) when ring Y is a 3- to 6-membered heterocycloalkyl ring, the 3- to 6-membered heterocycloalkyl ring is a 4-membered heterocycloalkyl ring containing one N heteroatom;
(4) when R⁵ and R⁶ are independently C₂₋₆ alkynyl or C₂₋₆ alkynyl substituted by one or more than one R⁵⁻¹, the C₂₋₆ alkynyl in the C₂₋₆ alkynyl and C₂₋₆ alkynyl substituted by one or more than one R⁵⁻¹ is ethynyl;
(5) when R⁵ and R⁶ are independently 5- to 6-membered heteroaryl or 5- to 6-membered heteroaryl substituted by one or more than one R⁵⁻², the 5- to 6-membered heteroaryl in the 5- to 6-membered heteroaryl and the 5- to 6-membered heteroaryl substituted by one or more than one R⁵⁻² is 5-membered heteroaryl containing 2 N heteroatoms;
(6) when R⁵⁻¹ and R⁵⁻² are independently C₁₋₆ alkyl, the C₁₋₆ alkyl is methyl;
(7) when R³ is 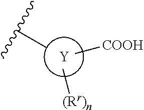
and ring Y is a 3 to 6-membered cycloalkyl ring, the 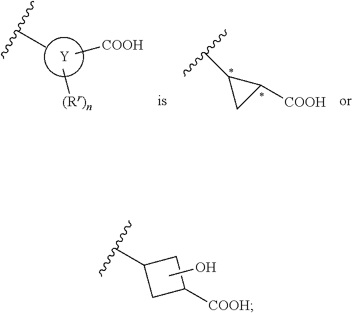
“*” means S configuration, R configuration, or a mixture of both.

5. The imidazole compound of formula I or the pharmaceutically acceptable salt thereof according to claim 1, wherein the imidazole compound of formula I satisfies one or more than one of the following conditions:
(1) when 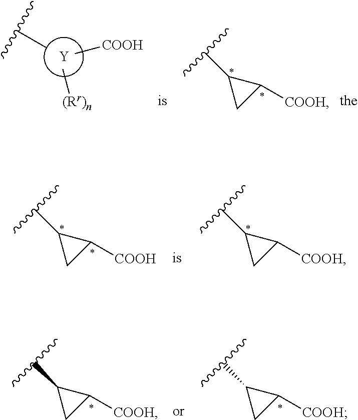
(2) when 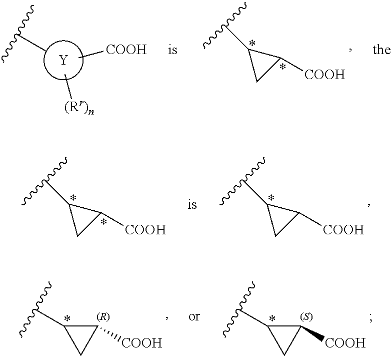
(3) when 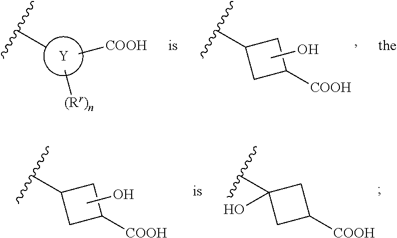
(4) when R³ is 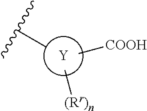
and ring Y is a 3- to 6-membered heterocycloalkyl ring, the 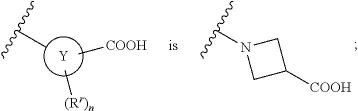
(5) when 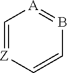
is a pyridine ring, 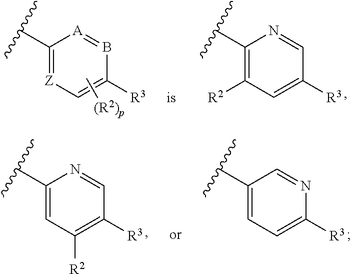
(6) when 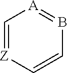
is a pyrimidine ring, 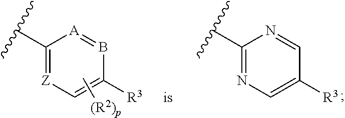
(7) when 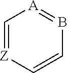
is a pyridazine ring, 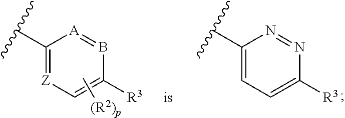
(8) when 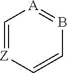
is a benzene ring,

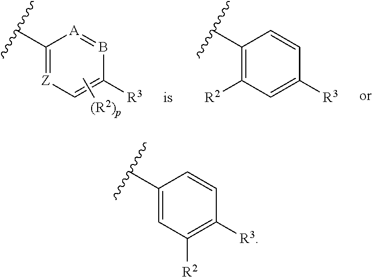

6. The imidazole compound of formula I or the pharmaceutically acceptable salt thereof according to claim 5, wherein the imidazole compound of formula I satisfies one or more than one of the following conditions:
(1) when 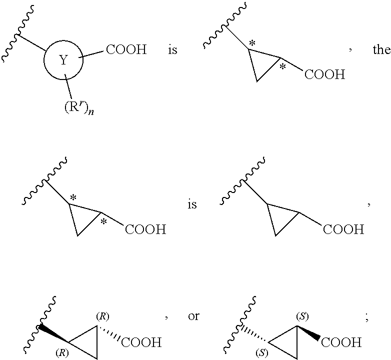
(2) when R¹ is fluorine and 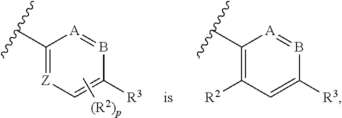
the R² is halogen;
(3) when R¹ is fluorine and 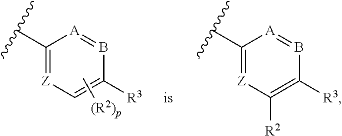
the R² is halogen or C₁₋₆ alkyl;
(4) when R¹ is chlorine and 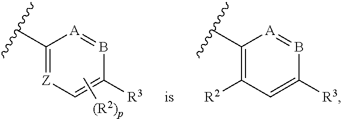
the R² is chlorine or C₁₋₆ alkyl;
(5) when R¹ is chlorine and 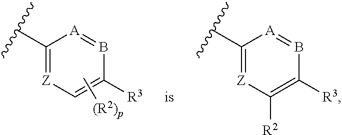
the R² is fluorine;
(6) when R¹ is H, 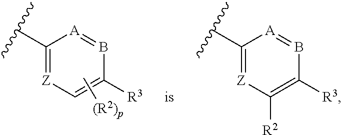
and R² is halogen or C₁₋₆ alkyl, the R² is chlorine or C₁₋₆ alkyl;
(7) when R¹ is H, 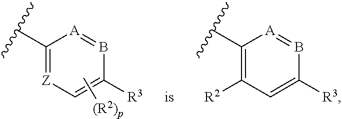
and R² is halogen or C₁₋₆ alkyl, the R² is halogen.

7. The imidazole compound of formula I or the pharmaceutically acceptable salt thereof according to claim 5, wherein the imidazole compound of formula I satisfies one or more than one of the following conditions:
(1) when R¹ is chlorine and 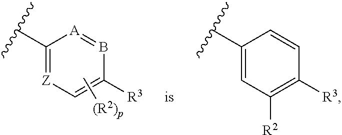
the R² is fluorine;
(2) when R¹ is chlorine and 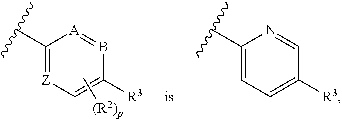
the R³ is 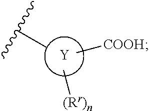
(3) when R¹ is H, 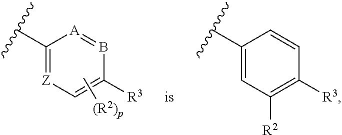
and R³ is 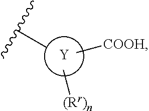
the R² is H, chlorine, or C₁₋₆ alkyl;
(4) when R¹ is H and 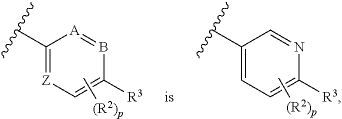
the R³ is 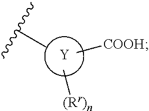
(5) when R¹ is H and 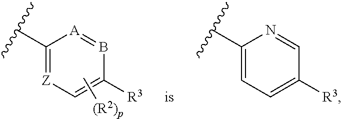
the R³ is

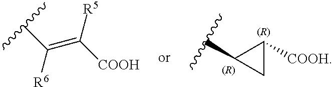

8. The imidazole compound of formula I or the pharmaceutically acceptable salt thereof according to claim 5, wherein the imidazole compound of formula I satisfies one or more than one of the following conditions:
(1) when R¹ is H and 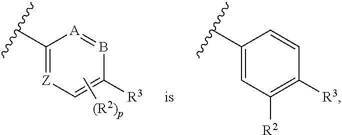
the R² is C₁₋₆ alkyl;
(2) when R¹ is H and 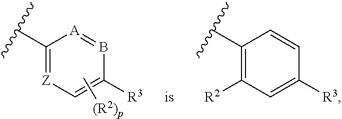
the R² is fluorine;
(3) when R¹ is fluorine or chlorine, and 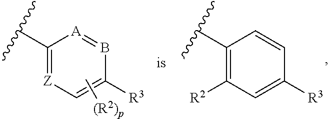
the R² is chlorine;
(4) when R¹ is chlorine or fluorine, and 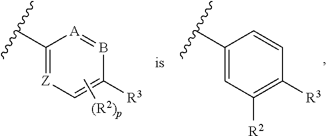
the R² is fluorine;
(5) when R¹ is H and 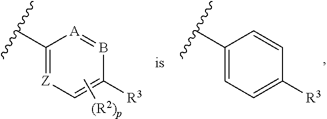
the R³ is 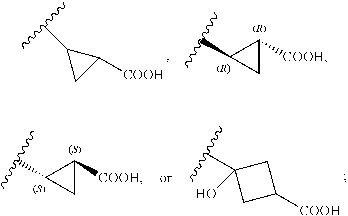
(6) when R¹ is H and 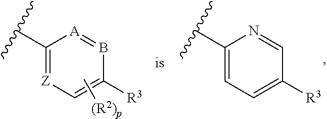
the R³ is 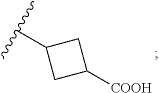 [SMILES: *C1CC(C(=O)O)C1]
(7) when R¹ is chlorine and R³ is

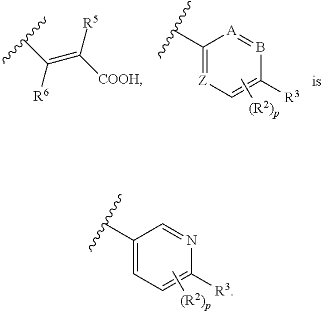

9. The imidazole compound of formula I or the pharmaceutically acceptable salt thereof according to claim 5, wherein the imidazole compound of formula I satisfies one or more than one of the following conditions:
(1) when R¹ is chlorine and 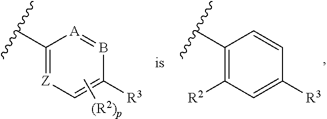
the R² is C₁₋₆ alkyl or halogen;
(2) when R¹ is fluorine and 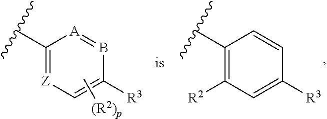
the R² is C₁₋₆ alkyl or chlorine;
(3) when R¹ is fluorine and 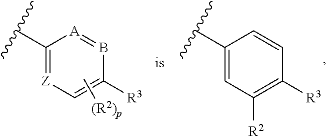
the R² is C₁₋₆ alkyl;
(4) when R¹ is H and 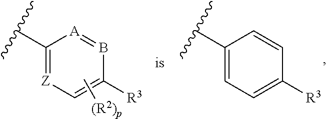
the R³ is 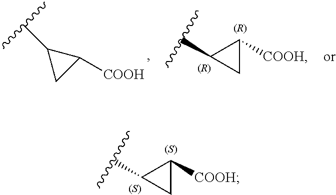
(5) when R¹ is H and 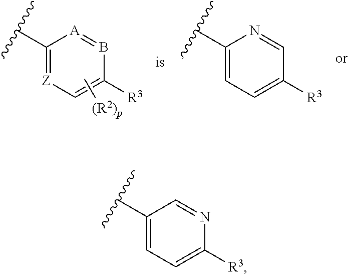
the R³ is

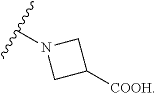 [SMILES: *N1CC(C(=O)O)C1]

10. The imidazole compound of formula I or the pharmaceutically acceptable salt thereof according to claim 1, wherein the imidazole compound of formula I satisfies any one of the following schemes:
Scheme 1:
in 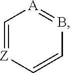
A, B, and Z are all CH, or at least one selected from the group of A, B, and Z is N;
R¹ is H or halogen;
the 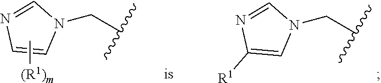
R² is H, halogen, or C₁₋₆ alkyl;
R³ is 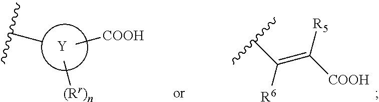
ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
R⁵ is independently H, C₂₋₆ alkynyl, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when A, B, and Z are CH at the same time, R⁵ and R⁶ are not H at the same time;
Scheme 2:
in 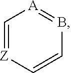
A, B, and Z are all CH, or at least one selected from the group of A, B, and Z is N;
R¹ is independently H or halogen;
the 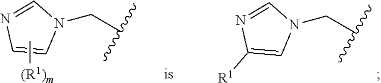
R² is 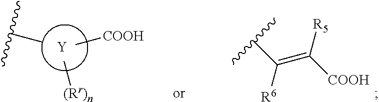
ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is 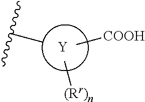
and ring Y is a 3- to 6-membered cycloalkyl ring, the 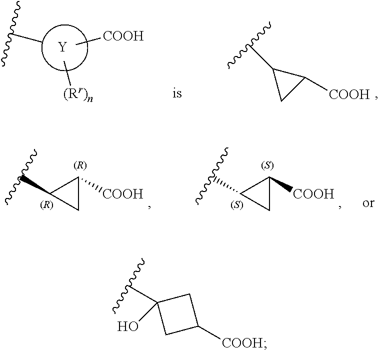
when R³ is 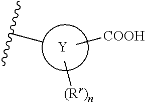
and ring Y is a 3- to 6-membered heterocycloalkyl ring, the 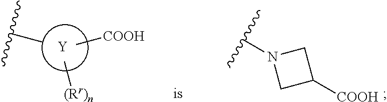
R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine and 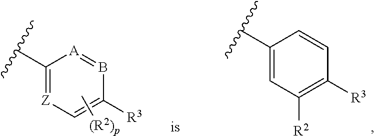
the R² is fluorine;
when R¹ is chlorine and 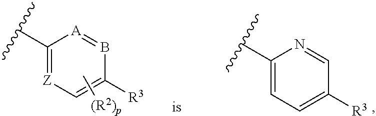
the R³ is 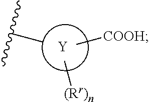
when R¹ is H, 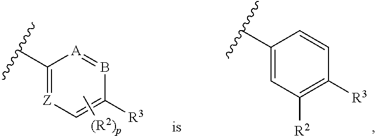
and R is 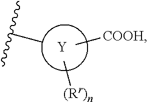
the R² is H, chlorine, or C₁₋₆ alkyl;
when R¹ is H and 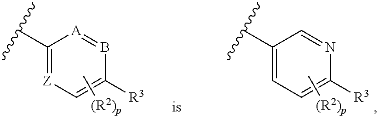
the R³ is 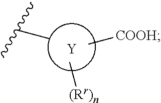
when R¹ is H and 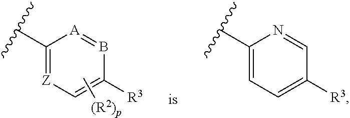
the R³ is 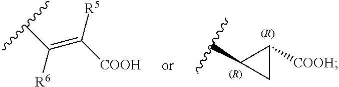
when A, B, and Z are CH at the same time, R⁵ and R⁶ are not H at the same time;
Scheme 3: 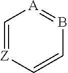
is a benzene ring, a pyridine ring, a pyrimidine ring, or a pyridazine ring;
R¹ is independently H or halogen;
the 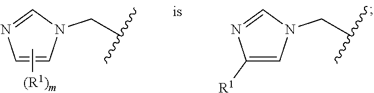
R² is H, halogen, or C₁₋₆ alkyl;
R³ is 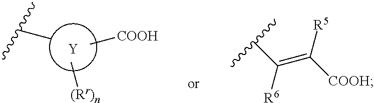
ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is 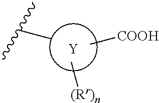
and ring Y is a 3- to 6-membered cycloalkyl ring, the 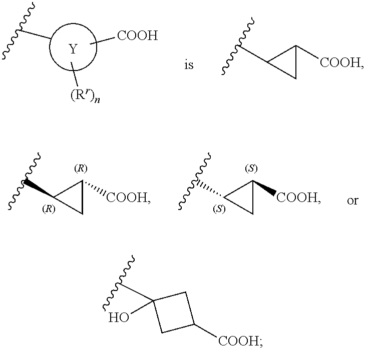
when R³ is 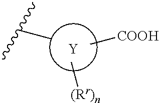
and ring Y is a 3- to 6-membered heterocycloalkyl ring, the 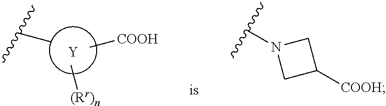
R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is H and 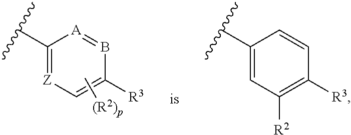
the R² is C₁₋₆ alkyl;
when R¹ is H and 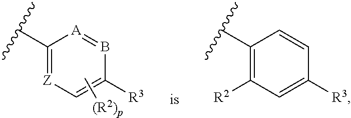
the R² is fluorine;
when R¹ is fluorine or chlorine, and 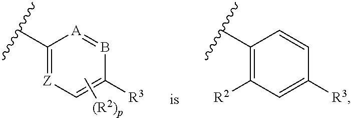
the R² is chlorine;
when R¹ is chlorine or fluorine, and 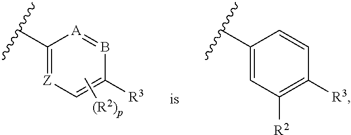
the R² is fluorine;
when R¹ is H and 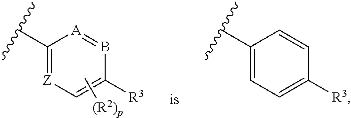
the R³ is 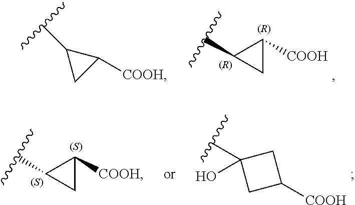
when R¹ is H and 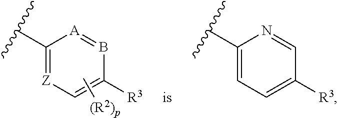
the R³ is 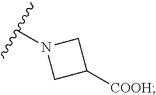 [SMILES: *N1CC(C(=O)O)C1]
when R¹ is chlorine and R³ is 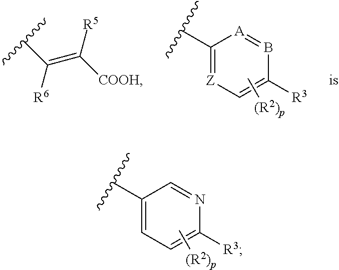
when A, B, and Z are CH at the same time, R⁵ and R⁶ are not H at the same time;
Scheme 4: 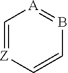
is a benzene ring, a pyridine ring, a pyrimidine ring, or a pyridazine ring;
R¹ is independently H or halogen;
the 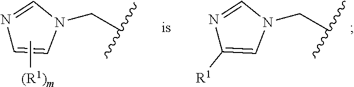
R² is H, halogen, or C₁₋₆ alkyl;
R³ is 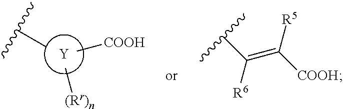
ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is 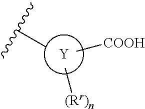
and ring Y is a 3- to 6-membered cycloalkyl ring, the 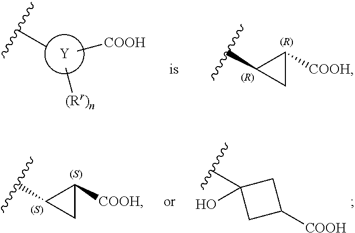
when R³ is 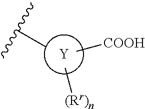
and ring Y is a 3- to 6-membered heterocycloalkyl ring, the 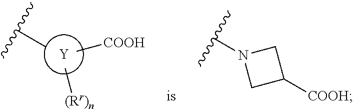
R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine and 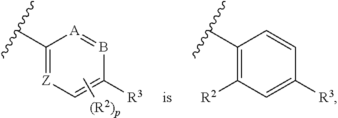
the R² is C₁₋₆ alkyl or halogen;
when R¹ is fluorine and 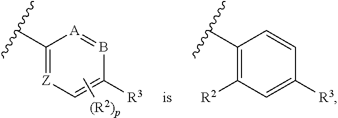
the R² is C₁₋₆ alkyl or chlorine;
when R¹ is fluorine and 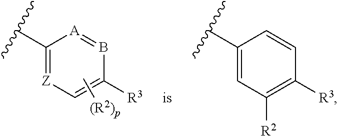
the R² is C₁₋₆ alkyl;
when R¹ is H and 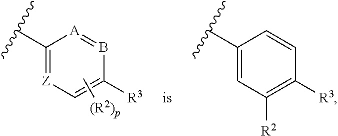
the R² is C₁₋₆ alkyl;
when R¹ is H and 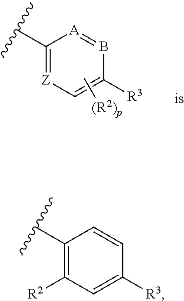
the R² is fluorine;
when R¹ is chlorine or fluorine, and 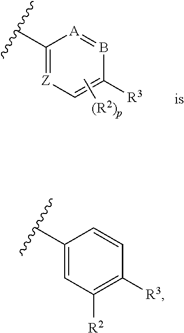
the R² is fluorine;
when R¹ is H and 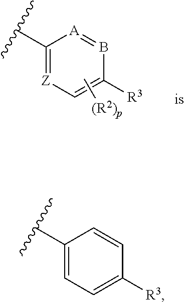
the R³ is 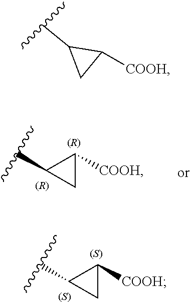
when R¹ is H and 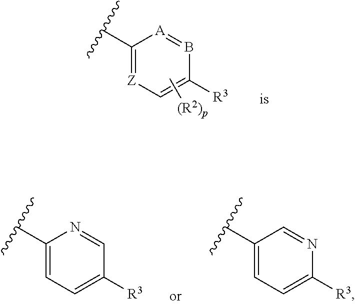
the R³ is 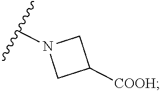 [SMILES: *N1CC(C(=O)O)C1]
when R¹ is chlorine and R³ is 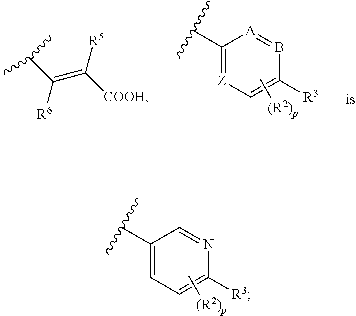
when A, B, and Z are CH at the same time, R⁵ and R⁶ are not H at the same time;
Scheme 5: 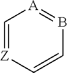
is a benzene ring, a pyridine ring, a pyrimidine ring, or a pyridazine ring;
R¹ is independently H or halogen;
the 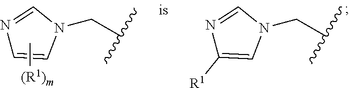
R² is H, halogen, or C₁₋₆ alkyl;
R³ is 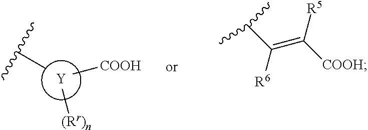
ring Y is a 3- to 6-membered cycloalkyl ring or a 3- to 6-membered heterocycloalkyl ring, and the 3- to 6-membered heterocycloalkyl ring is a 4-membered heterocycloalkyl ring containing one N heteroatom;
m, p, and n are independently 0 or 1;
R^{r} is H or —OH;
when R³ is 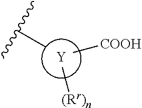
and ring Y is a 3- to 6-membered cycloalkyl ring, the 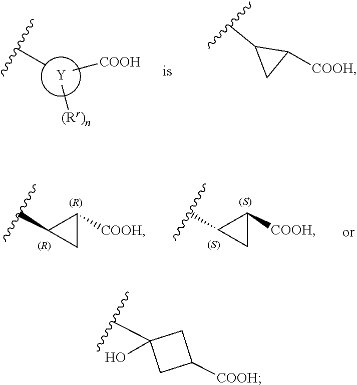
when R³ is 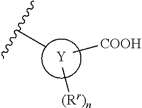
and ring Y is a 3- to 6-membered heterocycloalkyl ring, the 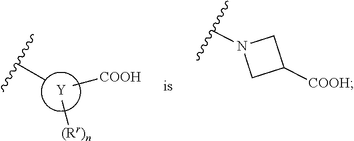
R⁵ is independently H, C₂₋₆ alkynyl substituted by one or more than one R⁵⁻¹, or 5- to 6-membered heteroaryl, and the 5- to 6-membered heteroaryl is 5-membered heteroaryl containing two N heteroatoms;
R⁵⁻¹ is independently C₁₋₆ alkyl;
R⁶ is H;
when R¹ is chlorine or fluorine, and 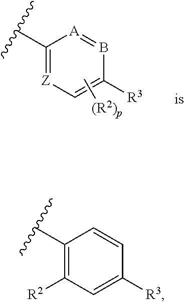
the R² is chlorine;
when R¹ is H, 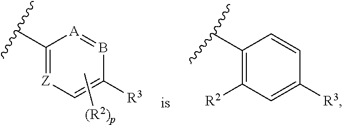
and R² is C₁₋₆ alkyl or halogen, the R² is fluorine;
when R¹ is H and 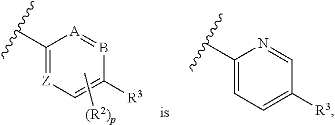
the R³ is 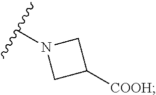 [SMILES: *N1CC(C(=O)O)C1]
when A, B, and Z are CH at the same time, R⁵ and R⁶ are not H at the same time;
Scheme 6: 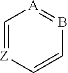
is a benzene ring;
R¹ and R² are independently H, chlorine, or fluorine;
the 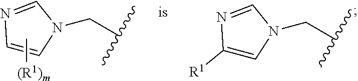
m and p are independently 0 or 1;
R³ is 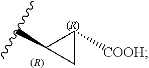
when R¹ is chlorine or fluorine, and 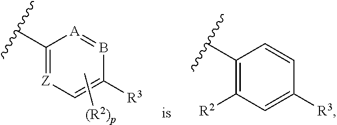
the R² is chlorine;
when R¹ is chlorine or fluorine, and 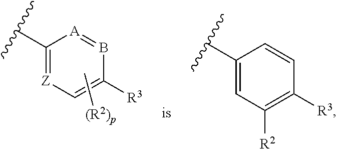
the R² is fluorine;
when R¹ is H and 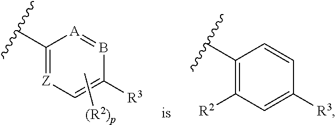
the R² is H or fluorine.

11. The imidazole compound of formula I or the pharmaceutically acceptable salt thereof according to claim 1, wherein the imidazole compound of formula I satisfies any one of the following schemes:
(1) 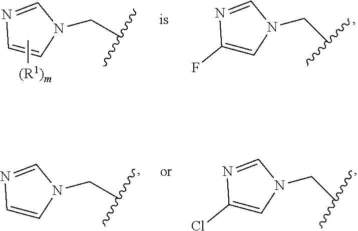
(2) when R³ is 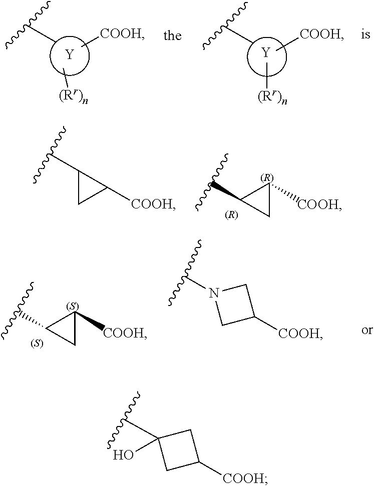
(3) when R³ is 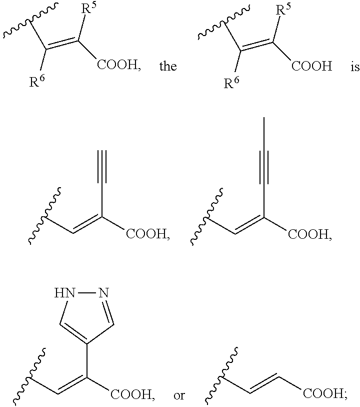
(4) 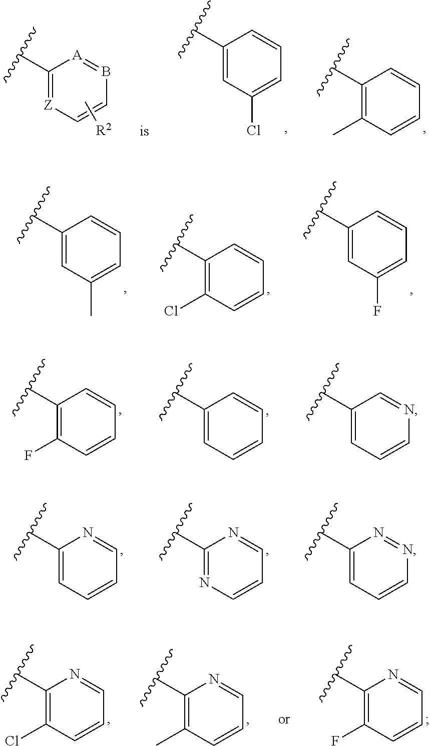
(5)

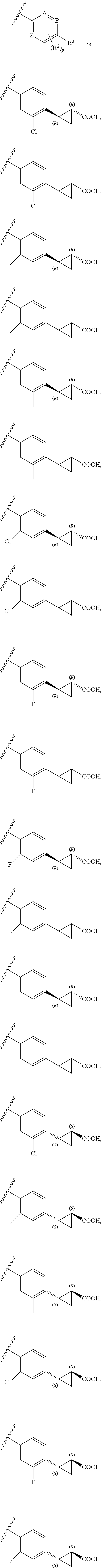

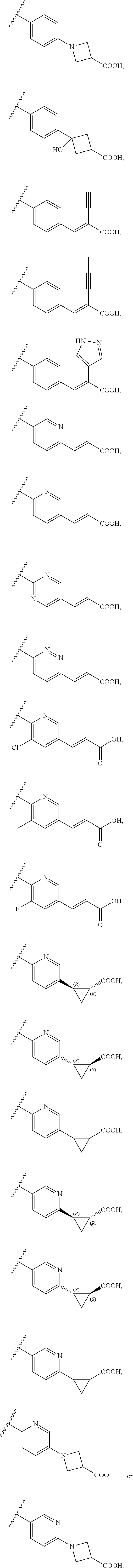

12. The imidazole compound of formula I or the pharmaceutically acceptable salt thereof according to claim 1, wherein the imidazole compound of formula I has any one of the following structures:

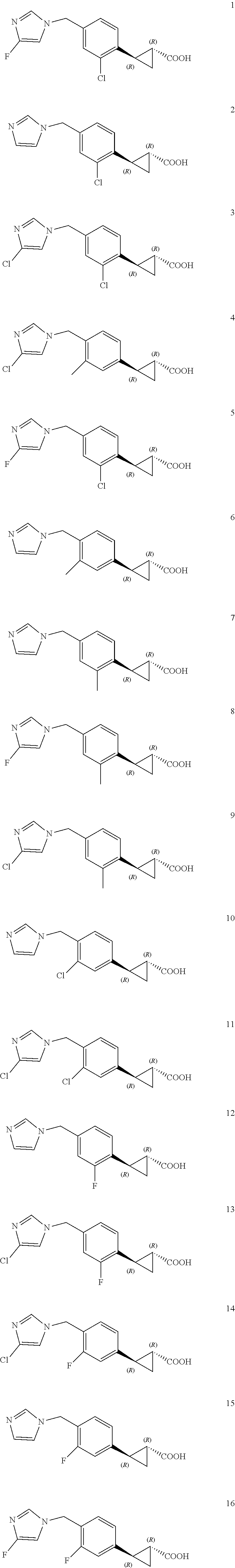

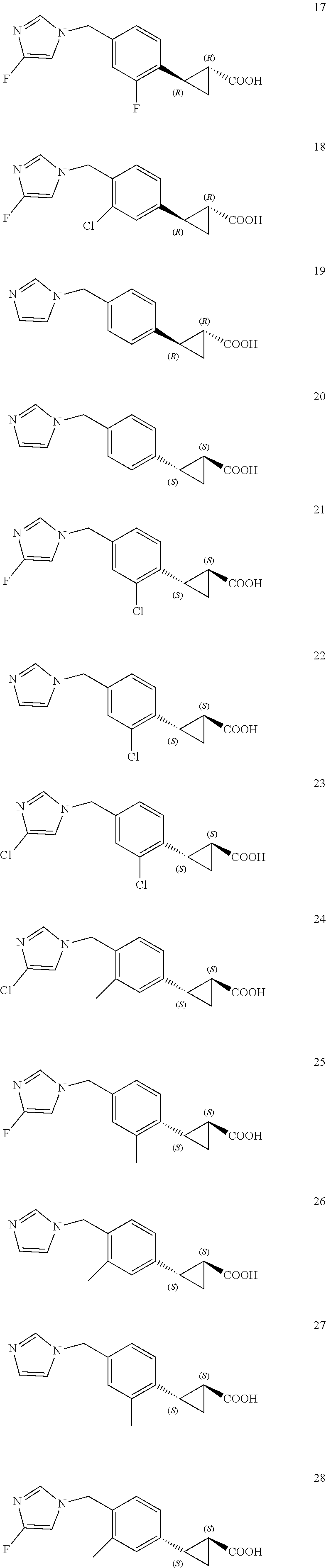

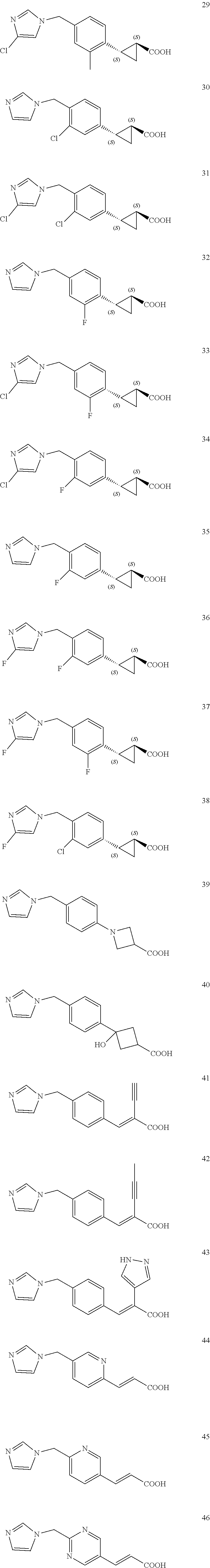

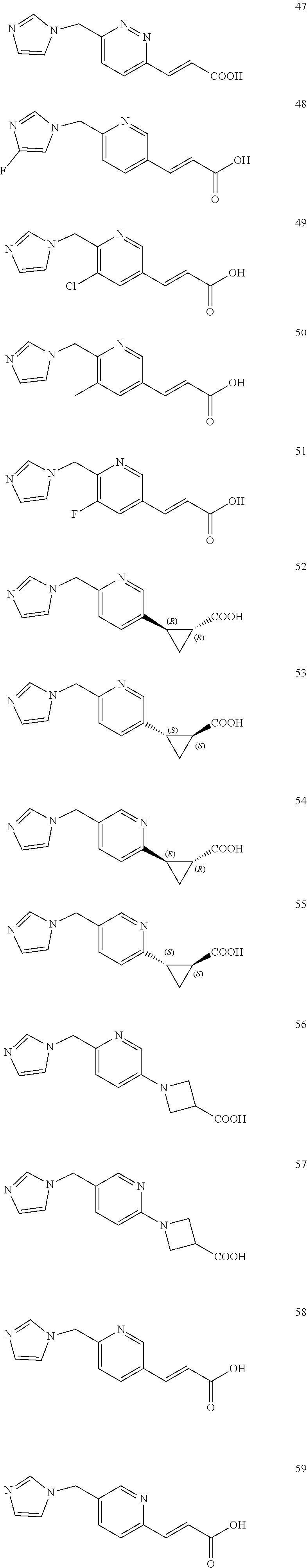

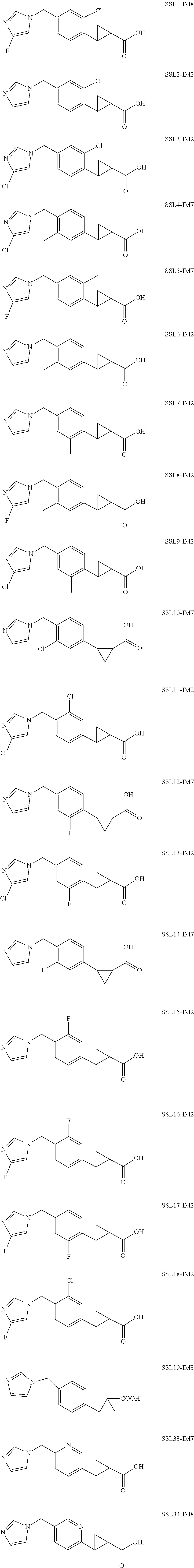

13. A compound of formula II or III, 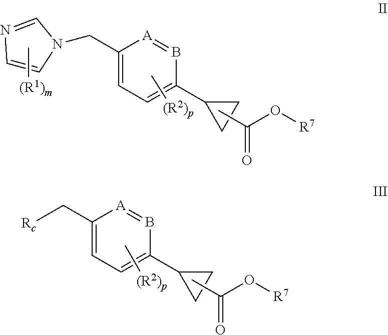
wherein A and B are independently CH or N:
R¹ and R² are each independently H, halogen, or C₁₋₆ alkyl;
m is 0, 1, 2, or 3;
p is independently 0, 1, 2, 3, or 4;
R_{c} is —OH, a leaving group, or an —O-hydroxyl protecting group;
R⁷ is C₁₋₆ alkyl.

14. The compound of formula II or III according to claim 13, wherein the compound of formula II or III satisfies one or more than one of the following conditions:
(1) the leaving group is Cl or Br;
(2) the hydroxyl protecting group is TBS;
(3) in R⁷, the C₁₋₆ alkyl is methyl, ethyl, n-propyl, isopropyl, n-butyl, sec-butyl, isobutyl, or tert-butyl, such as methyl or ethyl; the compound of formula II or III can be any one of the following compounds:

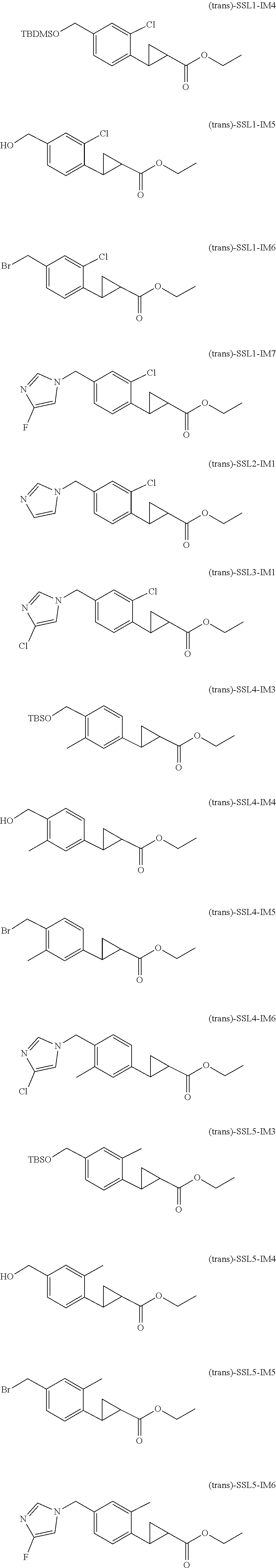

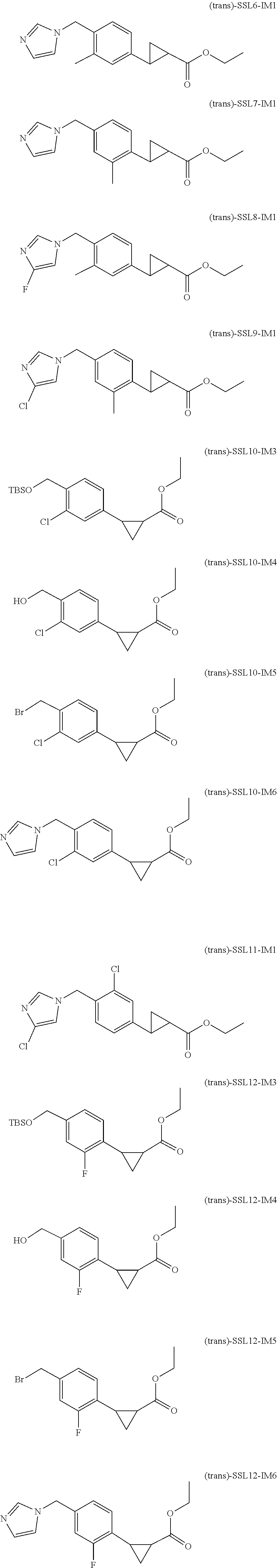

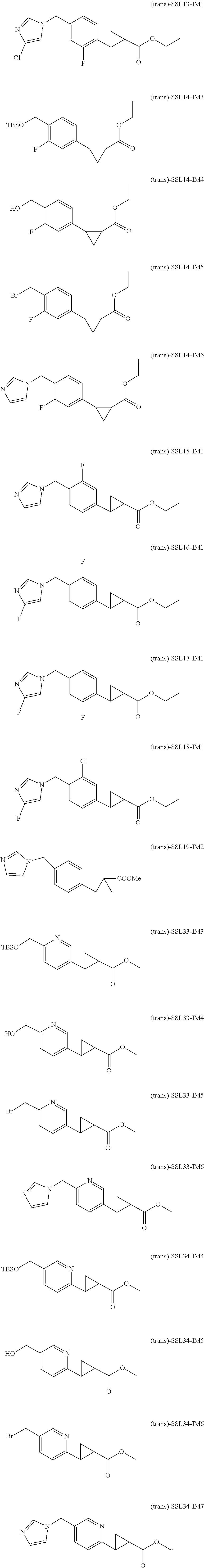

15. A pharmaceutical composition comprising substance A and a pharmaceutical excipient; the substance A is a therapeutically effective amount of the imidazole compound of formula I or the pharmaceutically acceptable salt thereof according to claim 1.

16. A method for inhibiting TXA₂ synthase in a subject in need thereof, comprising administering to the subject the imidazole compound of formula I or the pharmaceutically acceptable salt thereof according to claim 1; preferably, the method is used in mammalian organisms in vivo or used in vitro.

17. A method for treating or preventing a TXA₂-related disease in a subject in need thereof, comprising administering to the subject the imidazole compound of formula I or the pharmaceutically acceptable salt thereof according to claim 1; preferably, the TXA₂-related disease is a thrombotic disease, such as myocardial infarction, pulmonary embolism, or cerebral thrombosis.

18. A method for treating or preventing a thrombotic disease in a subject in need thereof, comprising administering to the subject the imidazole compound of formula I or the pharmaceutically acceptable salt thereof according to claim 1; preferably, the thrombotic disease is myocardial infarction, pulmonary embolism, or cerebral thrombosis.

19. A single crystal of a compound of formula A1 or a compound of formula A2, wherein
the structure data of the single crystal of the compound of formula A1 are as follows: 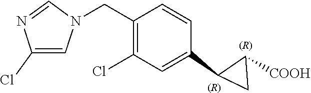
A1

| | Single crystal of the compound of | |
|---|---|---|
| Parameters | formula A1 | |
| Experimental formula | C14H12Cl2N2O2 | |
| Molecular weight | 311.16 | |
| Temperature | 113.15 | K |
| Wavelength | 0.71073 | A |
| Crystal system | Hexagonal | |
| Space group | P 61 | |
| Crystal cell size | a = 10.5982(3) Å, α = 90°. | |
| | b = 10.5982(3) Å, β = 90°. | |
| | c = 21.6750(8) Å, γ = 120°. | |
| Crystal cell volume | 2108.40(14) Å³ | |
| Z | 6 | |
| Density (calculated) | 1.470 | Mg/m³ |
| Absorption coefficient | 0.463 | mm⁻¹ |
| | |
|---|---|
| F(000) | 960.0 |
| Crystal size | 0.21 × 0.19 × 0.15 mm³ |
| θ range for data | 4.438 to 67.122° |
| collection | |
| Range of indicators | −16 ≤ h ≤ 16, −16 ≤ k ≤ 16, −29 ≤ l ≤ 32 |
| Diffraction point | 25979 |
| collection | |
| Independent diffraction | 5172 [Rint = 0.0599, Rsigma = 0.0513] |
| points | |
| Refinement method | Goodness-of-fit on F² |

. A
the structure data of the single crystal of the compound of formula A2 are as follows: 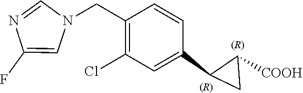
A2

| | Single crystal of the compound of | |
|---|---|---|
| Parameters | formula A2 | |
| Experimental formula | C14H12ClFN2O2 | |
| Molecular weight | 294.71 | |
| Temperature | 113.15 | K |
| Wavelength | 0.71073 | A |
| Crystal system | Monoclinic | |
| Space group | P 21 | |
| Crystal cell size | a = 4.5886(2) Å, α = 90°. | |
| | b = 10.9726(3) Å, β = 99.554(4)° | |
| | c = 13.3970(5) Å, γ = 90°. | |
| Crystal cell volume | 665.17(4) Å³ | |
| Z | 2 | |
| Density (calculated) | 1.471 | Mg/m³ |
| Absorption coefficient | 0.301 | mm⁻¹ |
| | |
|---|---|
| F(000) | 304.0 |
| Crystal size | 0.23 × 0.2 × 0.17 mm³ |
| θ range for data | 4.826 to 65.816° |
| collection | |
| Range of indicators | −6 ≤ h ≤ 6, −16 ≤ k ≤ 16, −20 ≤ l ≤ 19 |
| Diffraction point | 9980 |
| collection | |
| Independent diffraction | 4493 [Rint = 0.0310, Rsigma = 0.0400] |
| points | |
| Refinement method | Goodness-of-fit on F² |

20. The imidazole compound of formula I or the pharmaceutically acceptable salt thereof according to claim 4, wherein the imidazole compound of formula I satisfies one or two of the following conditions:
(1) when ring Y is a 3- to 6-membered heterocycloalkyl ring, the 3- to 6-membered heterocycloalkyl ring is 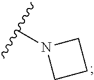 [SMILES: *N1CCC1]
(2) when R⁵ and R⁶ are independently 5- to 6-membered heteroaryl or 5- to 6-membered heteroaryl substituted by one or more than one R⁵⁻², the 5- to 6-membered heteroaryl in the 5- to 6-membered heteroaryl and the 5- to 6-membered heteroaryl substituted by one or more than one R⁵⁻² is pyrazolyl, such as 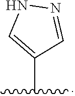 [SMILES: *c1cn[nH]c1]
